(19) United States
(12) Patent Application Publication
(10) Pub. No.: US 2011/0111430 A1
(43) Pub. Date: May 12, 2011

(54) METHOD FOR DIAGNOSING LIVER FIBROSIS

(75) Inventors: Ke X. Zhang, Thousand Oaks (CA); Wael A. Salameh, San Juan Capistrano (CA)

(73) Assignee: Quest Diagnostrics Investments, Inc.

(21) Appl. No.: 12/616,096

(22) Filed: Nov. 10, 2009

(51) Int. Cl. G01N 33/53; G06F 19/00

(52) U.S. Cl. 435/7.21; 436/86; 436/94; 702/19

(57) ABSTRACT

Provided herein are methods and devices for nonsurgically predicting, diagnosing, and monitoring liver fibrosis in an individual. Methods utilize biomarkers, age and sex to determine a diagnostic score. The diagnostic score is then used to predict, diagnose, or assess liver fibrosis in the individual.

### FIELD OF INVENTION

The present invention relates generally to the field of medical diagnostics and, more specifically, to the diagnosis of liver fibrosis.

### BACKGROUND OF THE INVENTION

Liver fibrosis is the formation or development of excess fibrous connective tissue in the liver characterized by the increased production and decreased degradation of extracellular matrix materials. Initiation of fibrosis formation is generally believed to occur through activation of Kupffer cells (macrophages which line the liver sinusoids) and subsequent secretion of multiple cellular factors. In addition, factors are secreted by damaged hepatocytes, thrombocytes, and endothelial cells of the hepatic sinusoid and other mediators. Collectively, these factors stimulate hepatic stellate cells, which differentiate into myofibroblasts, proliferating and synthesizing excess amounts of extracellular materials that gradually accumulate to develop liver fibrosis.

Liver fibrosis is common to liver diseases of many etiologies, including chronic viral hepatitis B and C, autoimmune liver disease, such as autoimmune hepatitis and primary biliary cirrhosis, alcoholic liver disease, nonalcoholic fatty liver disease, metabolic disorders, such as lipid, glycogen, or metal storage disorders, and drug-induced liver disease. The fibrosis exhibited in these disorders results from chronic insults to the liver from, for example, viral infection, alcohol, or drugs.

Hepatitis C, for example, is one of the leading causes of chronic liver disease in the United States, where an estimated 3.9 million people are chronically infected with hepatitis C virus (HCV) and approximately 30,000 new cases of acute HCV occur each year. Thus, the prevalence of hepatitis C is estimated to be 1.8% in the United States, with as many as 10,000 deaths per year resulting from chronic HCV infection (Alter, Semin. Liver Dis. 15:5-14 (1995)). World-wide, the prevalence of chronic HCV infection is estimated to be about 3% (J Viral Hepat 6:35-47 (1999)). Moreover, death, hospitalization and liver transplantation as a result of chronic hepatitis C have increased significantly in the past decade (Hepatology 36:S30-42 (2002)). Liver fibrosis is the main determinant of hepatitis C virus related morbidity and mortality (Lancet 349:825-323 (1997)). Furthermore, the stage of fibrosis is prognostic and provides information on the likelihood of disease progression and response to treatment (Hepatology 36:S47-564, 5 (2002); N Engl J Med 347:975-82 (2002)). The presence of significant fibrosis (equivalent to METAVIR F2 or greater) as determined by liver biopsy, is widely accepted as an indication to commence treatment (Gut 49:11-21 (2001); J Hepatol 31:3-8 (1999); Hepatology 39:1147-71 (2004)). The presence of cirrhosis has implications regarding screening for hepatocellular carcinoma and esophageal varices (J Hepatol 31:3-8 (1999)).

Liver biopsy is currently the gold standard for staging fibrosis. See, e.g., Gut 36:437-419, (1995); N Engl J Med 344:495-500 (2001); Hepatology 36:S47-564, 5 (2002); and Am J Gastroenterol 97:2614-8 (2002)). However, routinely measured serum markers, used either individually or in combination, have been examined as alternatives to liver biopsy for staging fibrosis among hepatitis C patients. Platelet count, ratio of aspartate aminotransferase (AST) to alanine aminotransferase (ALT), or a combination of AST and platelet count, are reliable predictors of cirrhosis (Arch Intern Med 163:218-24 (2003)). More complex models which include routinely available analytes such as cholesterol, γ-glutamyltransferase (GGT), platelet count, and prothrombin time, have demonstrated a high negative predictive value (NPV) for excluding significant hepatic fibrosis (Hepatology 39:1456-7 (2004)). A model incorporating measures of insulin resistance and past alcohol intake to reliably predict significant fibrosis has also been reported (Hepatology 39:1239-47 (2004)).

In efforts to improve the accuracy of noninvasive methods of staging liver fibrosis, several non-routinely-available biochemical markers associated with collagen and extra-cellular matrix deposition/degradation have been examined.

### SUMMARY OF INVENTION

The present invention provides nonsurgical methods of predicting the presence of liver fibrosis in an individual and methods for monitoring liver fibrosis in an individual. These methods include determining a single diagnostic score for the individual using the levels of three or more markers in a sample from the individual, including three or more of α2-macroglobulin (α2MG), hyaluronic acid (HA), matrix metalloproteinase-2 (MMP-2), and Activin A, and comparing the diagnostic score for the individual to a reference score (i.e., a cut-off value) to determine the presence or absence of liver fibrosis. In embodiments where the three or more markers do not include Activin A, then the plasma marker TIMP metallopeptidase inhibitor 1 (TIMP-1) is not used. In some embodiments, the methods are used to nonsurgically predict the presence of significant liver fibrosis.

In some embodiments, the diagnostic score is determined with three or more markers, and the individual's age. Other embodiments determine the diagnostic score using three or more markers and the individual's sex. In yet other embodiments, the diagnostic score is determined with three or more markers, the individual's age, and the individual's sex. In some embodiments, the three or markers comprise α2-macroglobulin (α2MG), hyaluronic acid (HA), and Activin A. In some embodiments, the three or markers comprise α2-macroglobulin, hyaluronic acid (HA), matrix metalloproteinase-2 (MMP-2), and Activin A.

In some embodiments, the diagnostic score is determined using a mathematical algorithm with variables representing three or more markers and variables representing one or both of the individual's age and sex.

In another aspect, the present invention provides nonsurgical methods of predicting the presence of liver fibrosis using a mathematical algorithm representing a Six Variable Model. This method is accomplished by obtaining a sample from an individual and determining the levels of four markers: α2MG, HA, MMP-2, and Activin A. The levels of these markers are input into a first equation (1), along with variables representing age and sex, to determine an intermediate value, y:

y=exp(X₁−(C₁*age)+(C₂*sex)+(C₃*α2MG)+(C₄*HA)+(C₅*MMP-2)+(C₆*Activin A)) (1)

wherein,
age is in years,
male sex=1, female sex=0,
α2MG is in mg/dL,
HA is in ng/mL,
MMP-2 is in ng/mL, and
Activin A is in pg/mL.

The intermediate value is input into a second equation (2) to calculate a diagnostic score, H:

H=y(1+y) (2)

The diagnostic score is compared to a cut-off value that is predictive of a disease or symptom. The constant (X₁) and coefficients (C₁, C₂, C₃, C₄, C₅, and C₆) in equation (1) can be varied across the following ranges: the constant, X₁, may be in the range of about −6.88236 to about −4.58824, such as about −6.30883 to about −5.16177; the age coefficient, C₁, may be in the range of about 0.05152 to about 0.07728, such as about 0.05796 to about 0.07104; the sex coefficient, C₂, may be in the range of about 0.45344 to about 0.68016, such as about 0.51012 to about 0.62348; the α2MG coefficient, C₃, may be in the range of about 0.00896 to about 0.01344, such as about 0.01008 to about 0.01232; the HA coefficient, C₄, may be in the range of about 0.00608 to about 0.00912, such as about 0.00684 to about 0.00836; the MMP-2 coefficient, C₅, may be in the range of about 0.00912 to about 0.01368, such as about 0.01026 to about 0.01254; and the Activin A coefficient, C₆, may be in the range of about 0.00216 to about 0.00324, such as about 0.00243 to about 0.00297. Preferably, the constant, X₁, is about −5.7353; the age coefficient, C₁, is about 0.0644; the sex coefficient, C₂, is about 0.5668; the α2MG coefficient, C₃, is about 0.0112; the HA coefficient, C₄, is about 0.00760; the MMP-2 coefficient, C₅, is about 0.0114; and the Activin A coefficient, C₆, is about 0.00270.

In another aspect, the invention provides methods of nonsurgical methods of predicting the presence of liver fibrosis using a mathematical algorithm representing a Five Variable Model. This method utilizes the levels of three markers: α2MG, HA, and Activin A. The levels of these markers are input into a first equation (3), along with variables representing age and sex, to determine an intermediate value, y:

y=exp(X₂−(C₇*age)+(C₈*sex)+(C₉*α2MG)+(C₁₀*HA)+(C₁₁*Activin A)) (3)

wherein,
age is in years,
male sex=1, female sex=0,
α2MG is in mg/dL,
HA is in ng/mL, and
Activin A is in pg/mL.

The intermediate value is input into a second equation (2) to calculate a diagnostic score:

H=y(1+y) (2)

The diagnostic score is compared to a cut-off value that is predictive of a disease or symptom. The constant (X₂) and coefficients (C₇, C₈, C₉, C₁₀, and C₁₁) in equation (3) can be varied across the following ranges: the constant, X₂, may be in the range of about −4.74948 to about −3.16632, such as about −4.35369 to about −3.56211; the age coefficient, C₇, may be in the range of about 0.04672 to about 0.07008, such as about 0.05256 to about 0.06424; the sex coefficient, C₈, may be in the range of about 0.38608 to about 0.57912, such as about 0.43434 to about 0.53086; the α2MG coefficient, C₉, may be in the range of about 0.0088 to about 0.01344, such as about 0.01008 to about 0.01232; the HA coefficient, C₁₀, be in the range of about 0.00672 to about 0.01008, such as about 0.00756 to about 0.00924; and the Activin A coefficient, C₁₁, be in the range of about 0.00288 to about 0.00432, such as about 0.00324 to about 0.00396. Preferably, the constant, X₂, is about −3.9579; the age coefficient, C₇, is about 0.0584; the sex coefficient, C₈, is about 0.4826; the α2MG coefficient, C₉, is about 0.0112; the HA coefficient, C₁₀, is about 0.0084; and the Activin A coefficient, C₁₁, is about 0.0036.

In another aspect, the invention provides methods of monitoring liver fibrosis in an individual by determining a diagnostic score of a first sample from the individual and determining a diagnostic score of a second sample from the individual, wherein the second sample was obtained from the individual at a time after obtaining the first sample. The levels in each sample of three or more of the markers α2MG, HA, MMP-2, and Activin A are used to determine diagnostic scores for each sample. Each diagnostic score is compared to a reference score (i.e., a cut-off value) to determine the presence or degree of liver fibrosis. The diagnostic scores of the first and second samples are then compared to determine progression or regression of liver fibrosis.

In one embodiment of the method for monitoring liver fibrosis, diagnostic scores for each sample are calculated using the Six Variable Model indicated above (equations (1) and (2)). In another embodiment of the method for monitoring liver fibrosis, diagnostic scores for each sample are calculated using the Five Variable Model indicated above (equations (3) and (2)).

In other embodiments of monitoring liver fibrosis, the presence or degree of liver fibrosis (e.g., significant fibrosis) as indicated by the first sample is compared to the presence or degree of liver fibrosis indicated by the second sample; wherein the an increase in the degree of liver fibrosis indicated by the second sample as compared with the first indicates progression of liver fibrosis whereas a decrease in the degree of liver fibrosis indicated by the second sample as compared to the first indicates a regression of liver fibrosis.

Some embodiments of monitoring liver fibrosis may be used to assess efficacy of liver fibrosis therapy. In these embodiments, no change or an increase in the degree of liver fibrosis indicated in the second sample as compared with the first indicates the liver fibrosis therapy is not efficacious, whereas a decrease in the degree of liver fibrosis indicated in the second sample as compared to the first indicates the liver fibrosis therapy is efficacious. In another embodiment for monitoring liver fibrosis therapy, a sample taken before initiation of treatment may be compared to samples taken after therapy has begun, including after therapy is concluded. Alternatively, two samples taken at different times during treatment may be compared or a sample taken during treatment may be compared with one taken after therapy is concluded.

In any of the foregoing aspects, a cut-off value is defined so that a diagnostic score above the cut-off value distinguishes between the presence and absence of significant fibrosis. For example, a diagnostic score greater than or equal to a cut-off value of about 0.5 on a scale of 0 to 1 is predictive of significant fibrosis. A diagnostic score less than a cut-off value of about 0.5 on a scale of 0 to 1 is predictive of an absence of significant fibrosis. In other embodiments, on a scale of 0 to 1, the cut-off value may be between about 0.425 to about 0.575, inclusive; such as between about 0.450 to about 0.550, inclusive; such as between about 0.475 to about 0.525, inclusive. Alternatively, on a scale of 0 to 1, the cut-off value may be about 0.425, about 0.450, about 0.475, about 0.5, about 0.525, about 0.550, or about 0.575. Optionally, individuals with a diagnostic score above a cut-off value are administered anti-fibrotic therapy. Thus, in some embodiments the diagnostic score is used to diagnose liver fibrosis.

Any of the foregoing methods may be used to predict the presence of liver fibrosis or diagnose liver fibrosis resulting from any disease including, for example, viral hepatitis, hepatitis B, hepatitis C, alcoholism and alcohol abuse, alcoholic liver disease, hemochromatosis, metabolic disease, diabetes, obesity, autoimmune hepatitis, nonalcoholic fatty liver disease, alcoholic fatty liver, drug-induced liver disease, primary biliary cirrhosis, primary sclerosing, cholangitis, α1-antitrypsin deficiency, Wilson disease, and chronic rejection or recurrent liver disease following liver transplantation.

In another embodiment, the foregoing methods are used to nonsurgically predict the presence of liver fibrosis in individuals infected with the hepatitis C virus. In other embodiments, the individual is co-infected with at least two viruses, including, for example, one or more of the following: hepatitis B, hepatitis C, hepatitis D and HIV-1.

In a particular aspect, provided herein is a device configured to nonsurgically predict the presence of liver fibrosis in an individual. The device comprises an input interface configured to receive data in data communication with a processor, which is in data communication with an output interface. In various embodiments the device could be a handheld device, computer, a laptop, portable device, a server, or the like.

The input interface is used for entry of data including levels of α2MG, HA, MMP-2, and Activin A as determined from a sample from the individual, and data for age and sex. Data may be entered manually by an operator of the system using an input interface such as a keyboard or keypad. Alternatively, data may be entered electronically, when the input interface is a cable in data communication with a computer, a network, a server, or analytical instrument. The input interface may wirelessly communicate with the processor.

The device further comprises a processor and a computer-readable storage medium including computer-readable instructions stored therein that, upon execution by the processor, cause the device to compute a diagnostic score, H. The diagnostic score, H, is computed using an algorithm. In some embodiments, the algorithm used to compute H is based on the Six Variable Model indicated above (i.e., equations (1) and (2)). In other embodiments, the algorithm used to compute H is based on the Five Variable Model indicated above (i.e., equations (3) and (2)).

In another embodiment, the device may further comprise readable instructions (e.g. software) stored on a computer-readable storage medium (e.g. memory) that, upon execution by the processor, compares the diagnostic score to a cutoff value to nonsurgically predict the presence of liver fibrosis, wherein a diagnostic score greater than or equal to a cut-off value is predictive of significant fibrosis. A diagnostic score less than a cut-off value is predictive of an absence of significant fibrosis. Exemplary cut-off values for use in these embodiments are described above. The computer-readable instructions may be executable instructions such as program code.

In one embodiment, the data output interface, in data communication with the processor, receives the diagnosis or the diagnostic score from the processor and provides the prediction or the diagnostic score to the device operator. The output interface may be, for example, a video display monitor or a printer. The output interface may be wirelessly connected to the processor. In a particular embodiment, a single device may function as the input interface and the output interface. One example of this type of interface is where the display monitor also functions as a keypad or touchscreen.

The concentration units in which the markers are expressed may be provided in units other than the ones recited above. Such a change in units would generate an equivalent equation to determine the intermediate value and would result in different absolute values for the constant and coefficients.

The phrase “diagnostic score” indicates the score resulting from a calculation based on one or more formulae where a formula gives individual weight to each marker used (e.g. α2MG, HA, MMP-2, and/or Activin A) in the calculation for assessing liver fibrosis in an individual.

The diagnostic score, “H”, is a number calculated from an intermediate value using equation (2) and ranges from 0.0 to 1.0.

H=y(1+y) (2)

The diagnostic score is compared with a cut-off value in order to determine the extent of fibrosis. In particular embodiments, a diagnostic score greater than or equal to a cut-off value (preferably about 0.5) is predictive of significant fibrosis, or stage F2-F4. A diagnostic score less than a cut-off value (preferably about 0.5) is predictive of the absence of significant fibrosis, or stage F0-F1.

The phrase “reference score” as used herein is a predetermined score or value that is statistically predictive of a symptom or disease, or lack thereof.

The phrase “cut-off value” as used herein refers to a reference score, specifically a diagnostic score, that is statistically predictive of a symptom or disease or lack thereof.

Fibrosis is scored on the 5-point METAVIR scale as follows: F0—no fibrosis, F1—portal fibrosis alone, F2—portal fibrosis with rare septae, F3—portal fibrosis with many septae, F4—cirrhosis. “Significant fibrosis” corresponds to stages F2, F3, and F4, while “advanced fibrosis” corresponds to stages F3 and F4. Absence of significant fibrosis corresponds to stages F0 and F1.

The phrase “intermediate value” as used herein represents a value, y, calculated from the levels of the three or four markers, age, and sex using equation (1) (in the Six Variable Method) or equation (3) (in the Five Variable Method).

As used herein, the term “specificity” means the probability that a diagnostic method of the invention gives a negative result when the sample is not positive, for example, of significant fibrosis (i.e., stage F2-F4). Specificity is calculated as the number of true negative results divided by the sum of the true negatives and false positives. Specificity is essentially a measure of how well a method excludes those who do not have a disease or symptom (e.g., significant fibrosis).

The term “sensitivity,” as used herein, refers to the characteristic of a diagnostic test that measures the ability of a test to detect a disease (or symptom) when it is truly present. Thus, sensitivity is the proportion of all diseased patients for whom there is a positive test, and is determined as the number of true positives divided by the sum of true positives and false negatives.

The phrase “negative predictive value (NPV),” means the probability that an individual diagnosed as not having fibrosis actually does not have the disease. Negative predictive value can be calculated as the number of true negatives divided by the sum of the true negatives and false negatives. Negative predictive value is determined by the characteristics of the diagnostic method as well as the prevalence of fibrosis in the population analyzed.

The phrase “positive predictive value (PPV),” means the probability that an individual diagnosed as having fibrosis actually has the disease or symptom. Positive predictive value can be calculated as the number of true positives divided by the sum of the true positives and false positives. Positive predictive value is determined by the characteristics of the diagnostic method as well as the prevalence of fibrosis in the population analyzed.

The tetra “accuracy” indicates the overall agreement between the diagnostic method and the disease state. Accuracy is calculated as the sum of the true positives and true negatives divided by the total number of sample results and is affected by the prevalence of fibrosis in the population analyzed.

A “ROC” is a receiver operating characteristic and a “ROC curve” is a graphical plot of the sensitivity versus (1-specificity) for a binary classifier system as its discrimination threshold is varied. The “AUC” is the area under a ROC curve and has a value between 0 and 1, in which a higher AUC indicates a better predictive value for the model.

The term “monitoring” liver fibrosis as used herein refers to comparison of two samples taken at different times to either determine the progression of liver fibrosis in an individual or determine the efficacy of liver fibrosis therapy in an individual. This comparison may include comparing diagnostic scores of samples collected at different times, or comparing indication of significant fibrosis in the individual from samples collected at different times.

The term “therapy” as used herein refers to any manner of treatment of a disease or symptoms thereof. Therapy of liver fibrosis includes any accepted or experimental treatment. Therapy may include treatment or removal of the causal agent or treatment of the fibrosis with drug compounds or other therapeutic agents.

The terms “efficacy” or “efficacious” as used herein refers to the ability of a drug, therapy or treatment to relieve symptoms or eliminate the disease. A treatment is said to have efficacy if any of certain positive outcomes, for example, a regression of liver fibrosis, occur as a result of the treatment.

As used herein, the term “sample” refers to a biological specimen that may contain one or more markers useful for nonsurgically predicting the presence or degree of liver fibrosis (e.g. α2MG, HA, MMP-2, and Activin A) according to the methods described herein. For example, a sample may be blood, serum, plasma, urine, saliva or liver tissue.

One skilled in the art would understand that the levels of markers may be assayed in a single sample, from separate samples, or in a number of combinations of samples, provided that the samples are obtained on the same day. The separate samples may be the same type of sample (e.g., serum) or may be different types (e.g., serum and plasma). In a particular embodiment, α2MG, HA, MMP-2, and Activin A each are assayed in the same serum sample.

The term “coefficient” as used herein refers to a numerical factor that each variable (i.e., age, sex, α2MG, HA, MMP-2, and Activin A) is multiplied by in equation 1 (in the Six Variable Method) or in equation 3 (in the Five Variable Method).

The population to which the invention pertains is preferably individuals receiving tertiary care such as in a tertiary care setting, although individuals receiving primary and secondary care also can be evaluated using the invention methods. As used herein the term “primary care facility” means a facility that offers first-contact health care only. As used herein the term “secondary care” refers to services provided by medical specialists who generally do not have first contact with patients (e.g., cardiologist, urologists, dermatologists) such typically occurs in a local (or community) hospitals setting.

The term “disease” as used herein refers to an interruption, cessation, or disorder of body functions, systems, or organs and is characterized usually by a recognized etiologic agent(s), an identifiable group of signs and symptoms, or consistent anatomical alterations.

As used herein the terms “level” or “concentration” of a marker are used interchangeably and refer to the relative or absolute amount or activity (e.g., enzymatic activity) of the marker per unit volume measured by any direct or indirect method. The level of a marker may be determined using any appropriate method including those methods exemplified herein.

The term “about” as used herein in reference to numbers or quantitative measurements, refers to the indicated value plus or minus 10%.

### BRIEF DESCRIPTION OF THE FIGURES

FIG. 1 is a series of receiver operating characteristic (ROC) curves showing stepwise logistic regression analysis of multivariate predictive models using various marker combinations.

FIG. 2 shows ROC curves for the Five Variable Model for sample sets. The data for these ROC curves are found in Table 9A-D. FIG. 2A: Receiver operating characteristic (ROC) curves for predicting significant fibrosis in patients with chronic hepatitis C: training set (n=181) compared to validation set (n=187). FIG. 2B: Receiver operating characteristic (ROC) curves for predicting significant fibrosis in patients with chronic hepatitis C: entire patient population (training+validation sets) (n=368) compared to “Singlets” population (subjects with more than one elevated analyte) (n=268). The five variables used in the Five Variable Method were age, sex, HA, α2MG, and Activin A.

FIG. 3 shows ROC curves for the Six Variable Model for sample sets. The receiver operating characteristic (ROC) curves are for predicting significant fibrosis in patients with chronic hepatitis C. The data for these ROC curves are found in Tables 11A-D. FIG. 3A: Training set (n=181). FIG. 3B: Validation set (n=187). FIG. 3C: Entire patient population (training+validation sets) (n=368). FIG. 3D: “Singlets” population (subjects with more than one elevated analyte) (n=268). The six variables used in the Six Variable Method were age, sex, HA, α2MG, MMP-2, and Activin A.

FIG. 4 compares ROC curves for the whole population to Singlets for the Six Variable Model. The receiver operating characteristic (ROC) curves for predicting significant fibrosis in patients with chronic hepatitis C compare the entire patient population (training+validation sets) (n=368) to “Singlets” population (subjects with more than one elevated analyte) (n=268). The six variables used in the Six Variable Method were age, sex, HA, α2MG, MMP-2, and Activin A.

FIG. 5 depicts box plots for the Six Variable Model according to fibrosis stage (F0-4) in all-data (n=368). The diagnostic score ranged from 0.0-0.1 and the fibrosis was staged according to METAVIR. The middle line inside the box represents the median. Inferior and superior ends of the boxes represent 25^{th} and 75^{th} percentile respectively. The whiskers are the 25^{th} and 75^{th} percentile ±1.5×IQR (interquantile range). The dots represent outliers. The six variables used in the Six Variable Method were age, sex, HA, α2MG, MMP-2, and Activin A.

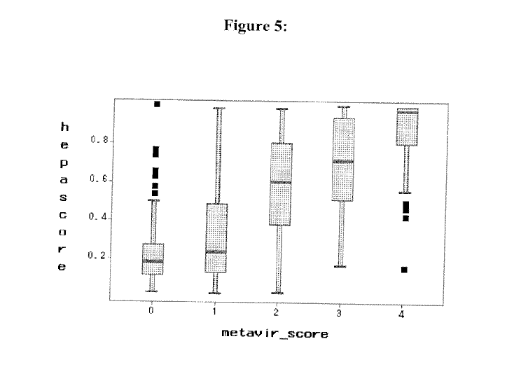

### DETAILED DESCRIPTION OF THE INVENTION

The methods provided here utilize levels of liver fibrosis markers present in one or more biological samples from an individual to calculate a diagnostic score. Comparison of the diagnostic score to a cut-off value provides indication of the presence or absence of significant liver fibrosis.

Marker levels used in the provided methods may be measured by a user, or provided to the user by an external source. Assays for detection of markers useful in the invention are well known in the art and in many cases are commercially available. Such assays include, but are not limited to, immunoassays such as radioimmunoassays, enzyme-linked immunosorbent assays (ELISA or EIA), two-antibody sandwich assays, quantitative western analysis, competitive and non-competitive immunoassays, antigen capture ELISA, immunonephelometry, and immunoturbidity; amplification based methods such as reverse transcription-PCR and other methods for quantitative analysis of RNA levels; and assays for biological activity such as enzymatic activity. Enzyme-linked protein binding assays may also be used. Enzyme-linked immunosorbent or protein-binding assays may be linked to a variety of enzymes such as horseradish peroxide (HRP), alkaline phosphatase (AP), beta-galactosidase, or urease. Detection methods can be coupled to chemiluminescent detection, flourescent detection or radioactive isotopes. Other gene expression assays may also be used for detecting levels of markers, such as Taqman or quantitative real-time PCR. Enzyme activity assays include, but are not limited to, gelatin zymography. These assays are well-known in the art and a person of ordinary skill in the art would understand which type of assay could be applied to a particular marker in order to measure the level of that marker. Accordingly, one or more of the following measurement methods may be used in some embodiments: the level of α₂-macroglobulin protein in a sample may be determined using an anti-α₂-macroglobulin antibody or by measuring the ability of α₂-macroglobulin to inhibit protease enzymatic activity; the level of hyaluronic acid in a sample may be determined using a hyaluronic acid-binding protein or using a an anti-hyaluronic acid antibody; the level of matrix metalloproteinase-2 protein in a sample may be determined using an anti-matrix metalloproteinase-2 antibody or by measuring the level of matrix metalloproteinase-2 enzymatic activity; and the level of Activin A protein in a sample may be determined using an anti-Activin A antibody.

Methods of the invention can be practiced by detecting and measuring the markers α2MG, HA, MMP-2 and Activin A, without measuring additional markers, or can be combined with a detection method for measuring one or more additional markers. Thus, in alternative embodiments, the invention could be practiced by detecting three or more of the markers α2MG, HA, MMP-2 and Activin A and also detecting at least one of the following markers of fibrosis: GGT, bilirubin, and MMP-1.

Discussion of exemplary methods of marker level determination are included below.

### Determination of Marker Levels

α₂-Macroglobulin

α₂-Macroglobulin (α2MG) is a high molecular weight protein found in plasma and binds several proteinases. α2MG contains a “bait region” that binds a protease, shielding it from macromolecular substrates but allowing reaction with small substrates and inhibitors. (Sottrup-Jensen L. et al., J Biol Chem; 264 (20):11539-11542 (1989)). These broad-spectrum protease inhibitors are synthesized in hepatocytes and stellate cells. (Naveau S et al., Dig Dis Sci.; 39:2426-32 (1994)).

The preferred method for determining α₂-macroglobulin (α2MG) levels is by nephelometry using the IMMAGE Immunochemistry System (Beckman Coulter). Nephelometry is commonly used to determine levels of IgM, IgG and IgA by measuring the amount of light scattered off of a sample. In this method, α2MG in the sample and an antibody against α2MG applied to the sample form α2MG-antibody aggregates. The IMMAGE system measures the rate of increase in light scattered from particles suspended in solution as a result of complexes formed during the above α2MG-antibody reaction. Reagents for this assay are provided with the IMMAGE system and the assay is run per the manufacturer's protocol. Briefly, reagents, calibrator (Beckman Calibrator 2), controls and samples are loaded into the system. The automated system adds 21 μL anti-α2MG antibody, 300 μL Buffer 1, and 20.42 μL Diluent 1 to 0.58 μL sample. The system calculates the level of α2MG protein in the sample and reports the result in g/L.

In addition to nephelometry, a variety of other assays for detecting and measuring levels of α2MG are known in the art and include direct and indirect assays for detecting α2MG RNA, α2MG protein and α2MG activity. Various detection methods are well-known in the art and include, for example, immunoassays, including radioimmunoassay (RIA), enzyme-linked immunoassay (ELISA), fluorescence immunoassay (FIA), two-antibody sandwich assays, and immunoturbidity. Monoclonal and polyclonal anti-α2MG antibodies useful in immunoassays can be readily obtained from a variety of sources.

A variety of assays for indirect determination of the level of α2MG in a sample may be used. For example, a level of α2MG may be determined as a function of inhibition of target protease activity, without a corresponding inhibition of amidolytic activity. α2MG can be detected and the level of α2MG can be determined by assaying for inhibition of trypsin, subtilisin, chymotrypsin, plasmin, elastase, thermolysin, or papain activity without inhibition of amidolytic activity. (Armstrong et al., Develop. Compar. Immunol. 23:375-390 (1999)). The level of α2MG can also be determined by assaying for inhibition of the activity of two or more proteases with different active site specificities, for example, two or more of the following proteases: trypsin, subtilisin, chymotrypsin, plasmin, elastase, thermolysin and papain. (Armstrong et al., Develop. Compar. Immunol. 23:375-390 (1999)). Another method of detecting and determining the levels of α2MG that may be used is based on α2MG's ability to shield a bound protease from an antibody or a high molecular weight inhibitor. (Ganorot, Clin. Chem. Acta 14:493-501 (1966); Armstrong et al., Develop. Compar. Immunol. 23:375-390 (1999)).

α2MG may also be detected, or an α2MG level can be determined, by analysis of α2MG mRNA levels using routine techniques such as Northern analysis, RT-PCR, or methods based on hybridization to a nucleic acid sequence that is complementary to a portion of the α2MG coding sequence. For example, conditions and probes for Northern analysis and RNA slot blot hybridization of α2MG RNA in 20 human samples are described in Ortego et al., Exp. Eye Res. 65:289-299 (1997), and Simon et al., Cancer Res. 56:3112-3117 (1996), respectively. In addition, α2MG levels may be detected using quantitative real time PCR. Exemplary sequence of the genomic DNA comprising the α2MG gene can be found in NCBI GenBank, accession number M11313, the sequence of which is incorporated herein by reference. (Kan et al., Proc. Natl. Acad. Sci. 82:2282-2286 (1985)).

Hyaluronic Acid

Hyaluronic acid (HA), also known as hyaluronate or hyaluronan, is a high molecular weight polysaccharide with an unbranched backbone comprised of dimeric units consisting of glucuronic acid and β-(1,3)-N-acetylglucosamine moieties connected by β-1,4 linkages. Hyaluronic acid can have a length of a few such dimeric units to more than 1,000, with each dimeric unit having a molecular weight of about 450 D. Hyaluronic acid is produced primarily by fibroblasts and other specialized connective tissue cells and plays a structural role in the connective tissue matrix. Hyaluronic acid is widely distributed throughout the body and can be found as a free molecule in, for example, plasma, synovial fluid, and urine.

Levels of HA are preferably determined using an enzyme-linked protein binding assay commercially available assay from Corgenix (Westminster, Colo.). This test is a sandwich protein binding assay which employs hyaluronic acid binding protein (HABP) as the capture molecule. In this assay, diluted serum or plasma and HA reference solutions are incubated in HABP-coated microwells. HA present in samples is captured by the immobilized binding protein (HABP). Unbound serum components are removed by washing, and HABP conjugated with horseradish peroxidase (HRP) solution is added to the microwells and complexes with bound HA. Unbound conjugated HABP is removed by washing and a chromogenic substrate of tetramethylbenzidine and hydrogen peroxide is added to develop a colored reaction. The intensity of the color is measured in optical density (O.D.) units with a spectrophotometer at 450 nm. Optical density is converted to HA concentration using a standard curve.

Other methods of determining hyaluronic acid levels may be used provided detection is comparable to that obtained by the preferred method. Such methods are well-known in the art and include, for example, a variety of competitive and non-competitive binding assays and immunoassays. Competitive binding assays using ¹²⁵I-labeled HA binding protein; competitive binding assays based on alkaline phosphatase labeled-hyaluronectin (HN); and non-competitive binding assays based on peroxidase-labeled proteoglycan or peroxidase-labeled HA-binding protein, among others, are well-known in the art. See, for example, Lindquist et al., Clin. Chem. 38:127-132 (1992); Delpech and Bertrand, Anal. Biochem. 149:555-565 (1985); Engstrom-Laurent et al., Scand. J. Clin. Lab. Invest. 45:497-504 (1985); Brandt et al., Acta Otolaryn. 442 (Suppl.):31-35 (1987); Goldberg, Anal. Biochem. 174:448-458 (1988); Chichibu et al., Clin. Chim. Acta 181:317-324 (1989); Li et al., Conn. Tissue Res. 19:243-254 (1989); Poole et al., Arth. Rheum. 33:790-799 (1990); Poole et al., J. Biol. Chem. 260:6020-6025 (1985); and Laurent and Tengblad, Anal. Biochem. 109:386-394 (1980)). A variety of immunoassay formats may be used to determine a level of HA, including radioimmunoassay (RIA), enzyme-linked immunoassays (EIA), and fluorescence immunoassay (FIA). Polyclonal or monoclonal anti-HA antibodies useful in immunoassays are commercially available from a variety of sources.

Matrix Metalloproteinase-2

Matrix metalloproteinase-2 (MMP-2), also known as gelatinase A, is an extracellular matrix degradative enzyme. Part of a larger family of matrix metalloproteinases, MMP-2 is involved in the breakdown of extracellular matrix in the physiological processes of the normal liver. MMPs control deposition of extracellular matrix by remodeling matrix components such as collagens, fibronectin, laminin, tenascin, undulin and entactin. (Cawston et al., “Protein Inhibitors of Metalloproteinases” in Barrett and Salvesen (Eds), Proteinase Inhibitors Amsterdam Elsevier 45 pages 589-610 (1986)).

MMP-2 levels may be determined using a quantitative sandwich enzyme immunoassay kit (R&D systems). Other methods of determining MMP-2 levels may be used provided detection is comparable to that obtained by the preferred method. Such methods are well-known in the art and include, for example, other immunoassays, including radioimmunoassay (RIA), fluorescence immunoassay (FIA) and two-antibody sandwich assays.

MMP-2 levels may also be measured using enzymatic activity assays known in the art. One such assay is gelatin zymography, where gelatin is used as a substrate for demonstrating the activity of gelatin-degrading proteases, such as MMP-2. (Ratnikov B, et al. Laboratory Investigation 82:11 (2002)). Zymography is an electrophoretic technique, based on SDS-PAGE, where the substrate is copolymerized with the polyacrylamide gel, for the detection of enzyme activity. Samples are prepared in the standard SDS-PAGE treatment buffer but without boiling, and without a reducing agent. Following electrophoresis, the SDS is removed from the gel (or zymogram) by incubation in unbuffered Triton X-100, followed by incubation in an appropriate digestion buffer, for an optimized length of time at 37° C. The zymogram is subsequently stained (commonly with Amido Black or Coomassie Brilliant Blue), and areas of digestion appear as clear bands against a darkly stained background where the substrate has been degraded by the enzyme. (Lantz M S, et al. Methods Enzymol. 235: 563-594 (1994)).

MMP-2 can also be detected and the levels determined by analysis of MMP-2 mRNA levels using routine techniques such as Northern analysis, RT-PCR, or methods based on hybridization to a nucleic acid sequence that is complementary to a portion of the MMP-2 coding sequence. For example, probes for Northern analysis and RNA slot blot hybridization of MMP-2 RNA may be used and techniques are well-known in the art. The sequence of the 13 exons of the MMP-2 gene can be found in NCBI GenBank, accession number J05471, the sequence of which is incorporated herein by reference. (Huhtala, et al. J. Bio. Chem. 265:19:11077-11082 (1990)).

Activin A

Activin A is one of a group of Activins that are polypeptide hormones (i.e. cytokines) belonging to the transforming growth factor-β (TGF-β) superfamily. The TGF-β superfamily is a large group of extra-cellular growth factors which control many aspects of development, reproductive function and tumor formation. (Chang H et al. Endocrine Rev 23: 787-823, 2002)). Activin A is a homo-dimeric protein complex made up of two beta A subunits linked by a single covalent disulfide bond. Each subunit, also called inhibin β A, is encoded by the InhbA gene. Activin A has important regulatory functions in reproductive biology, embryonic development, inflammation and tissue repair. (Rodgarkia-Dara C, et al. Mutat Res. 2006 November-December; 613 (2-3):123-37. Epub 2006 Sep. 25). Activin A binds to the proteins follistatin and α₂-macroglobulin. (Krummen L A et al. Endocrinology 132: 431-443 (1993)). It has also been found to regulate cell number in the liver by inhibiting hepatocyte replication and induction of apoptosis. It stimulates extracellular matrix production in hepatic stellate cells and tubulogenesis of sinusoidal endothelial cells, and contributes to restoration of tissue architecture during liver regeneration. (Deli A, et al. World J Gastroenterol. 21:14 (11): 1699-709 (2008)).

Activin A levels may be determined using an enzyme-linked immunosorbent assay (Diagnostic Systems Laboratories, Inc.). Other methods of determining Activin A levels may be used provided detection is comparable to that obtained by the preferred method. Such methods are well-known in the art and include, for example, other immunoassays, including radioimmunoassay (RIA), fluorescence immunoassay (FIA), and two-antibody sandwich assays. An enzyme-linked protein binding assay using an Activin A binding protein, such as Follistatin, could also be used to determine levels of Activin A.

Determination of a Diagnostic Score

In particular aspects, the levels of the markers as determined above, along with age and sex, are input into either the Six Variable Model or the Five Variable Model.

Six Variable Model

The Six Variable Model utilizes generic equation (1) to determine an intermediate value, y:

y=exp(X₁−(C₁*age)+(C₂*sex)+(C₃*α2MG)+(C₄*HA)+(C₅*MMP-2)+(C₆*Activin A)) (1)

wherein,
age is in years,
male sex=1, female sex=0,
α2MG is in mg/dL,
HA is in ng/mL,
MMP-2 is in ng/mL, and
Activin A is in pg/mL.

The values of the constant (X₁) and the coefficients (C₁, C₂, C₃, C₄, C₅, and C₆) may be determined by analysis of a training set to give the best agreement with known results. Generally, the constant (X₁) and coefficients (C₁, C₂, C₃, C₄, C₅, and C₆) will be in the following ranges: the constant, X₁, may be in the range of about −6.88236 to about −4.58824, such as about −6.30883 to about −5.16177; the age coefficient, C₁, may be in the range of about 0.05152 to about 0.07728, such as about 0.05796 to about 0.07104; the sex coefficient, C₂, may be in the range of about 0.45344 to about 0.68016, such as about 0.51012 to about 0.62348; the α2MG coefficient, C₃, may be in the range of about 0.00896 to about 0.01344, such as about 0.01008 to about 0.01232; the HA coefficient. C₄, may be in the range of about 0.00608 to about 0.00912, such as about 0.00684 to about 0.00836; the MMP-2 coefficient, C₅, may be in the range of about 0.00912 to about 0.01368, such as about 0.01026 to about 0.01254; and the Activin A coefficient, C₆, may be in the range of about 0.00216 to about 0.00324, such as about 0.00243 to about 0.00297. Preferably, the constant, X₁, is about −5.7353; the age coefficient, C₁, is about 0.0644; the sex coefficient, C₂, is about 0.5668; the α2MG coefficient, C₃, is about 0.0112; the HA coefficient, C₄, is about 0.00760; the MMP-2 coefficient, C₅, is about 0.0114; and the Activin A coefficient, C₆, is about 0.00270.

Implementation of the preferred values for the constant and coefficients gives the following equation (1a) for an intermediate value, y:

y=exp(−5.7353−(0.0644*age)+(0.5668*sex)+(0.0112*α2MG)+(0.00760*HA)+(0.0114*MMP-2)+(0.00270*Activin A)) (1a)

where age, sex, α2MG, HA, MMP-2, and Activin A are defined as above.

The diagnostic score, H, is calculated using the intermediate value, y, in the equation (2):

H=y(1+y) (2)

Five Variable Model

The Five Variable Model utilizes generic equation (3) to determine an intermediate value, y:

y=exp(X₂−(C₇*age)+(C₈*sex)+(C₉*α2MG)+(C₁₀*HA)+(C₁₁*Activin A)) (3)

wherein,
age is in years,
male sex=1, female sex=0,
α2MG is in mg/dL,
HA is in ng/mL, and
Activin A is in pg/mL.

The values of the constant (X₂) and the coefficients (C₇, Cₛ, C₉, C₁₀, and C₁₁) may be determined by analysis of a training set to give the best agreement with known results. Generally, the constant (X₂) and coefficients (C₇, C₈, C₉, C₁₀, and C₁₁) will be in the following ranges: the constant, X₂, may be in the range of about −4.74948 to about −3.16632, such as about −4.35369 to about −3.56211: the age coefficient, C₇ may be in the range of about 0.04672 to about 0.07008, such as about 0.05256 to about 0.06424; the sex coefficient, C₈, may be in the range of about 0.38608 to about 0.57912, such as about 0.43434 to about 0.53086; the α2MG coefficient, C₉, may be in the range of about 0.0088 to about 0.01344, such as about 0.01008 to about 0.01232; the HA coefficient, C₁₀, be in the range of about 0.00672 to about 0.01008, such as about 0.00756 to about 0.00924; and the Activin A coefficient, C₁₁, be in the range of about 0.00288 to about 0.00432, such as about 0.00324 to about 0.00396. Preferably, the constant, X₂, is about −3.9579; the age coefficient, C₇, is about 0.0584; the sex coefficient, C₈, is about 0.4826; the α2MG coefficient, C₉, is about 0.0112; the HA coefficient, C₁₀, is about 0.0084; and the Activin A coefficient, C₁₁, is about 0.0036.

Implementation of the preferred values for the constant and coefficients gives the following equation (3a) for the determination of an intermediate value, y:

y=exp(−3.9579−(0.0584*age)+(0.4826*sex)+(0.0112*α2MG)+(0.0084*HA)+(0.0036*Activin A)) (3a)

where age, sex, α2MG, HA, and Activin A are defined as above.

The diagnostic score, H, is calculated using the intermediate value, y, in the equation (2):

H=y(1+y) (2)

Prediction of Liver Fibrosis

The diagnostic score, H, is compared to a cut-off value, in order to identify significant fibrosis (METAVIR stages F2 to F4). A diagnostic score greater than or equal to a cut-off value (preferably about 0.5) is predictive of significant fibrosis, whereas, a diagnostic score less than a cut-off value (preferably about 0.5) is predictive of an absence of significant fibrosis.

Cut-off values may be determined by analysis of a training set to give the best agreement with known results. Cut-off values may be between about 0.425 to about 0.575, inclusive; such as between about 0.450 to about 0.550, inclusive; such as between about 0.475 to about 0.525, inclusive. Alternatively, this cut-off value may be about 0.425, about 0.450, about 0.475, about 0.5, about 0.525, about 0.550, or about 0.575. Optionally, individuals with a diagnostic score above a cut-off value are administered anti-fibrotic therapy.

### EXAMPLES

### Example 1

### Selection of Patient Population

Three hundred sixty eight patients with chronic HCV infection undergoing liver biopsy were enrolled from the Liver Center at Beth Israel Deaconess Medical Center, a tertiary referral center in Boston, Mass. All patients had chronic HCV infection as confirmed by HCV-RNA polymerase chain reaction analysis in serum, and none were on active antiviral treatment for HCV at time of biopsy. Coexisting liver diseases attributable to alcohol, hepatitis B, autoimmune hepatitis, primary biliary cirrhosis, hemochromatosis, α1-antitrypsin deficiency, or Wilson's disease were reasonably excluded by history and standard clinical, laboratory, imaging, and histologic studies. Human immunodeficiency virus coinfection and post-transplant patients were also excluded. The study was approved by the institutional review board at Beth Israel Deaconess Medical Center. All patients gave informed consent. Liver biopsies were performed as part of routine clinical care in the staging and grading of liver disease. Age and sex were recorded at the time of liver biopsy.

All 368 patients were randomly assigned to training or testing sets. Of the patients assigned to the training set (n=181), 96 patients (53%) had significant fibrosis (F2-4) and 85 patients (47%) did not have significant fibrosis (F0-F2). Of the patients assigned to the validation set (n=187), 89 patients (47.6%) had significant fibrosis and 98 patients (52.8%) did not have significant fibrosis.

### Example 2

### Assay of Markers

Serum was collected either at the time of liver biopsy or within 3 months of the biopsy. Standard laboratory assessments including complete blood cell count with platelets, serum chemistry panels, alanine aminotransferase test (ALT), aspartate aminotransferase (AST), GGT, and total bilirubin were performed by a licensed clinical laboratory on fresh serum within 36 hours of collection. Other analyses were performed on sera stored frozen at −80° C. Total bilirubin and GGT were measured on an AU640e instrument (Olympus Diagnostica, Center Valley, Pa.) by using the GGT reagent pack and the total bilirubin reagent pack (Olympus Diagnostica System), respectively. α2MG was measured on a BNII nephelometer (Dade Behring Inc, Marburg, Germany). MMP-1, MMP-2, Activin A and TIMP-1 were measured using enzyme-linked immunosorbent assays. HA was measured with an enzyme-linked protein-binding assay (Corgenix Inc, Denver, Colo.). All analyses were performed at Quest Diagnostics Nichols Institute, San Juan Capistrano, Calif. A summary of the methods of detection used for each analyte is presented in Table 1.

TABLE 1
Method of Detection for fibrotic markers.

| Marker | Method of Detection | Vendor |
|---|---|---|
| MMP-2 | EIA | R&D Systems |
| α2MG | Nephelometry | Immage, Beckman Coulter |
| Activin A | EIA | Oxford Bio./DSL |
| Hyaluronic acid | Enzyme linked protein | Corgenix, Inc |
| | binding assay | |

### Example 3

Analysis of Liver Biopsy. Liver biopsy samples were obtained under ultrasound guidance or marking with either a 16-gauge TruCut or 18-gauge Menghini needle. All biopsies were deemed adequate on the basis of either specimen size (≧10 mm) or the number of portal tracts (≧8). A single expert pathologist blinded to all clinical and serologic results evaluated all slides. Biopsies were interpreted according to the scoring schema developed by the METAVIR group. Fibrosis was scored on a 5-point scale: F0, no fibrosis; F1, portal fibrosis alone; F2, portal fibrosis with rare septae; F3, portal fibrosis with many septae; F4, cirrhosis. The presence of stage F2, F3, or F4 was termed significant fibrosis, whereas F3 or F4 was considered advanced fibrosis.

### Example 4

### Statistical Analysis

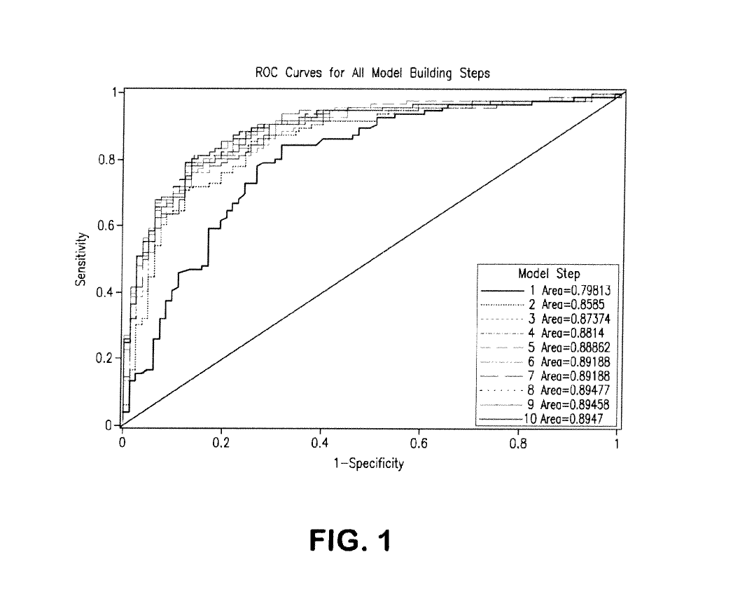

Using the data from the training set, associations between each biochemical marker and the presence or absence of significant fibrosis were assessed by logistic regression. In addition, the diagnostic accuracy of each biochemical marker was assessed using receiver operating characteristic (ROC) curve analysis. All markers were combined with age and sex and entered into stepwise logistic regression analysis using a forward and a backward elimination procedure with a significance level of P=0.30 (FIG. 1). The dependent variable was defined as significant fibrosis as detected by liver biopsy. Markers with a high AUC or a high level of significance on univariate analysis were added to create different multivariable models. Models based on different marker combinations, were then compared using receiver operating characteristic (ROC) curves to determine which was most accurate in detecting significant fibrosis. Ten variables, including age, sex, α2MG, HA, GGT, Bilirubin, Activin A, MMP-1, MMP-2, and TIMP-1 were evaluated with logistic regression analysis using the training set. See FIG. 1.

### Example 5

### Development of the Five Variable Model

Out of the ten variables tested, including age, sex, α2MG, HA, GGT, Bilirubin, Activin A, MMP-1, MMP-2, and TIMP-1, five variables were selected for development of a model. These five variable model were age, sex, α2MG, HA, and Activin A. Of the remaining markers, GOT, Bilirubin, MMP-1 and TIMP-1 were not included as they did not significantly improve sensitivity or specificity of the test. The results of statistical analysis of these variables for the training set and development of a constant and coefficients for the model are found in Table 2, where the column “Estimate” lists the estimated coefficients for each variable for the logistic regression model.

TABLE 2
Analysis of Parameter Estimates. Statistical results
for development of the Five Variable Model.

| | | | Standard | Likelihood | Wald | Pr > | Confidence |
|---|---|---|---|---|---|---|---|
| Parameter | DF | Estimate | Error | Ratio 95% | Chi-Square | ChiSq | Limits |
| Intercept | 1 | −3.9579 | 1.4838 | −7.018 | −1.1491 | 7.11 | 0.0076 |
| Age | 1 | −0.0584 | 0.0302 | −0.12 | −0.0006 | 3.74 | 0.053 |
| Sex | 1 | 0.4826 | 0.4484 | −0.389 | 1.3809 | 1.16 | 0.2818 |
| HA | 1 | 0.0084 | 0.004 | 0.0022 | 0.018 | 4.36 | 0.0369 |
| α2MG | 1 | 0.0112 | 0.0024 | 0.006 | 0.0163 | 21.86 | <.0001 |
| ActivinA | 1 | 0.0036 | 0.0013 | 0.0013 | 0.0064 | 7.91 | 0.0049 |

Thus, logistic regression analysis of the training set lead to the Five Variable Model:

y=exp(−3.9579−(0.0584*age)+(0.4826*sex)+(0.0112*α2MG)+(0.0084*HA)+(0.0036*Activin A)),

where age is in years; male sex=1, female sex=0, α2MG in mg/dL, HA in ng/mL and Activin A in pg/mL.

The diagnostic score, H, is calculated using the intermediate value, y, in the following equation:

H=y(1+y)

The ability of the model to predict significant fibrosis (F2-4) as determined by the area under the ROC curve was similar in training (0.884) and validation sets (0.885). The training plus validation sets gave an area under the ROC curve of 0.884. Sensitivity, specificity, PPV and NPV for significant fibrosis were determined for various cut-off points between zero and one, in 0.01 increments, in the training set, validation set, training plus validation sets and the singlets set (patients where only one analyte was elevated were excluded).

The full set of data for each sample set evaluated with the Five Variable Model is presented in Tables 10A-D below, which include accuracy, sensitivity, specificity, NPV, and PPV for each possible score at increments of 0.01 for sample sets (Training, Validation, Training+Validation, and Training+Validation excluding singlets).

The following labels are used in Tables 10A-D:

correct=number of patients correctly diagnosed with either Stage 0 (F0-F1) fibrosis or Stage 1 (F2-4) fibrosis.

incorrect=number of patients incorrectly diagnosed with either Stage 0 or Stage 1 fibrosis.

evt=number of patients diagnosed with Significant Fibrosis (Stage 1).

non-evt=number of patients diagnosed with absence of significant fibrosis (Stage 0).

accuracy=true positives+true negatives/total population

sensi=Sensitivity (y-axis of ROC curve)

spec=Specificity

1-Spec=1-Specificity (x-axis of ROC curve)

f-pos=false positives

f-neg=false negatives

PPV=positive predictive values

NPV=negative predictive values

Prevalence: Actual prevalence of population=53%; Scenarios of predicted higher and lower prevalence=10%, 70%, 50%

AUC=area under the ROC curve

### Example 6

### Results of Five Variable Model: Testing, Validation, and Singlets Sets

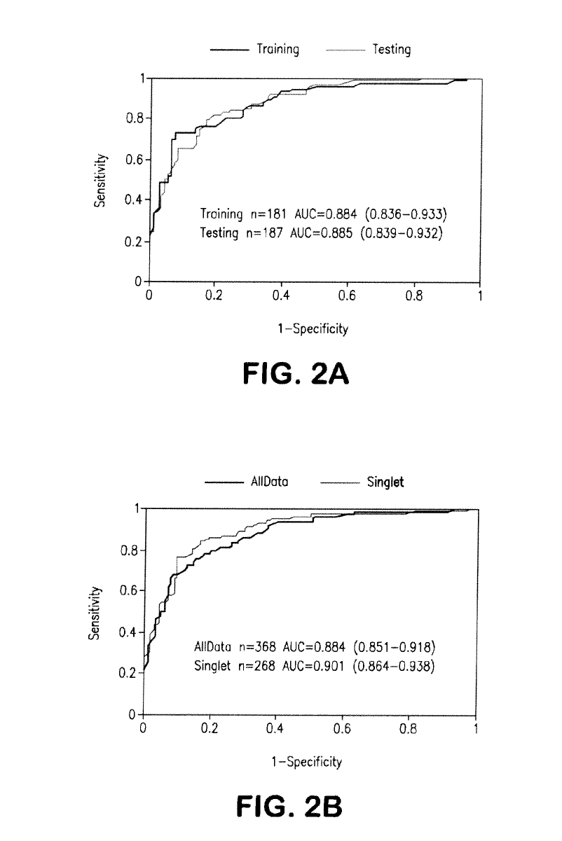

Using the constant and coefficients derived above, the diagnostic score cut-off for detecting significant fibrosis is about 0.50. Using a cut-off value of 0.50 for predicting significant fibrosis among the training set of 181 patients resulted in a sensitivity of 76% and a specificity of 81.2%. The accuracy in predicting significant fibrosis for this cut-off value was 78.5%. The area under the ROC curve was 0.884. See FIG. 2.

The Five Variable Model was applied to the validation set of 187 patients, resulting in an area under the curve of 0.885. Using a central cut-off point of 0.50 for predicting significant fibrosis among the validation set of 187 patients resulted in a sensitivity of 80.9% and a specificity of 81.6%. The accuracy in predicting significant fibrosis for this cut-off value was 81.3%.

When the Five Variable Model was applied to the training set plus the validation set (i.e., all 368 patients), the area under the ROC curve was 0.884. Using a cut-off value of 0.50 for predicting significant fibrosis among the validation set of 187 patients resulted in a sensitivity of 78.4% and a specificity of 81.4%. The accuracy in predicting significant fibrosis for this cut-off value was 79.9%.

Analysis using the Five Variable Model was also performed on the testing plus validation set and singlets (Testing+Validation excluding individuals with only one marker elevated). Within the total population (n=368), 268 individuals had only a single elevated analyte. The prevalence of significant fibrosis in the sample excluding singlets was 55%. By removing the singlets and reanalyzing the data, the predictive value of the Five Variable Model improved.

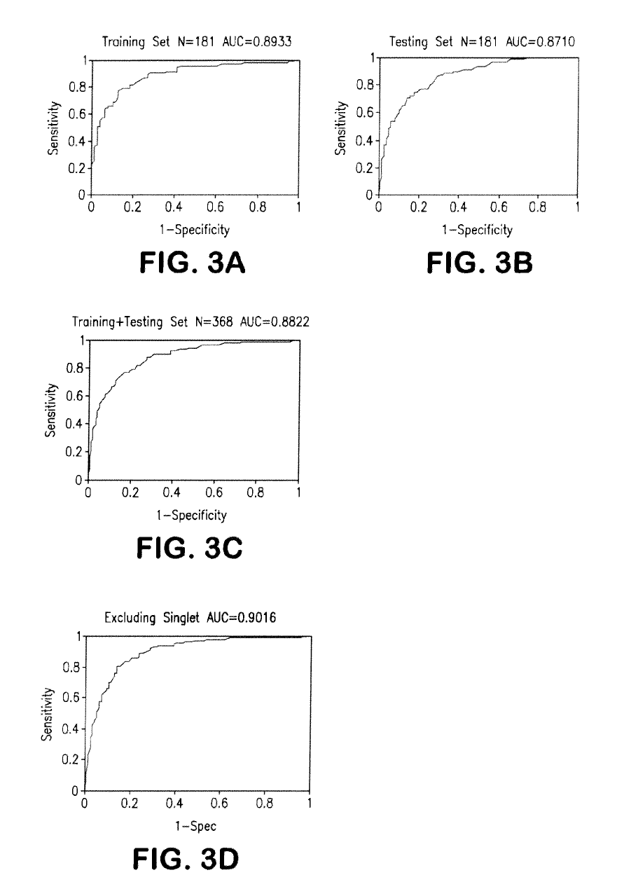

The ROC curve for the entire sample set (Testing+Validation) is compared with the Singlet sample set (Testing+Validation excluding singlets) in the graph in FIG. 3.

The probability of fibrosis at particular diagnostic scores was also evaluated for the entire population. Table 3 shows the probabilistic likelihoods for each fibrosis stage for individuals with diagnostic scores much higher (e.g., greater than or equal to 0.8) or lower (e.g., less than or equal to 0.3) than the cut-off value. For example, according to the data set, a patient with a diagnostic score of ≦0.1 has a 93.2% probability of being stage F0-1, and a 6.8% probability of being stage F2. Results of this analysis are presented in Table 3.

TABLE 3
Five Variable Model: Probabilities of Each Fibrosis Stage for High
and Low Diagnostic Scores (n = 368)
All-data n = 368

| | Diagnostic Score (H) | | | | |
|---|---|---|---|---|---|
| | ≦0.1 | ≦0.2 | ≦0.3 | ≧0.8 | ≧0.9 |
| Popu- | 44 | 91 | 130 | 100 | 70 |
| lation | | | | | |
| F0-1 | 41(93.2%) | 84 (92.3%) | 116 (89.2%) | 8 (8%) | 3 (4.3%) |
| F2-4 | 3 (6.8%) | 7 (7.7%) | 14 (10.8%) | 92 (92%) | 67 (95.7%) |
| F2 | 3 (6.8%) | 5 (5.5%) | 10 (7.7%) | 11 (11%) | 3 (4.3%) |
| F3 | 0 (0%) | 1 (1.1%) | 3 (2.3%) | 26 (26%) | 17 (24.3%) |
| F4 | 0 (0%) | 1 (1.1%) | 1 (0.77%) | 55 (55%) | 47 (67.1%) |

### Example 7

### Development of the Six Variable Model

Out of the ten variables tested, including age, sex, α2MG, hyaluronic acid, GGT, Bilirubin, Activin A, MMP-1, MMP-2, and TIMP-1, six variables were selected for development of a Six Variable Model. The six variables selected for inclusion in the Six Variable Model were age, sex, α2MG, HA, MMP-2, and Activin A. Again, GUT, Bilirubin, MMP-1 and TIMP-1 were not included as they did not significantly improve specificity or sensitivity. The results are found in Table 4, where the column “Estimate” lists the estimated coefficients for each variable for the logistic regression model.

TABLE 4
Analysis of Parameter Estimates. Statistical results for development
of the Six Variable Model.

| | | | Standard | Wald | |
|---|---|---|---|---|---|
| Parameter | DF | Estimate | Error | Chi-Square | Pr > ChiSq |
| Intercept | 1 | −5.7353 | 1.7894 | 10.2731 | 0.0013 |
| Age | 1 | −0.0644 | 0.0305 | 4.4554 | 0.0348 |
| Sex | 1 | 0.5668 | 0.4544 | 1.5563 | 0.2122 |
| HA | 1 | 0.0112 | 0.00243 | 21.1947 | <.0001 |
| MMP-2 | 1 | 0.00760 | 0.00418 | 3.3111 | 0.0688 |
| α2MG | 1 | 0.0114 | 0.00601 | 3.5943 | 0.0580 |
| ActivinA | 1 | 0.00270 | 0.00135 | 4.0112 | 0.0452 |

Thus, logistic regression analysis of the training set lead to the Six Variable Model:

y=exp(−5.7353−(0.0644*age)+(0.5668*sex)+(0.0112*α2MG)+(0.00760*HA)+(0.0114*MMP2)+(0.00270*Activin A))

where age is in years; male sex=1, female sex=0, α2MG in mg/dL, HA in ng/mL and Activin A is in pg/mL.

The diagnostic score, H, was calculated using the intermediate value, y, in the following equation:

H=y(1+y)

The ability of the model to predict significant fibrosis (F2-4) as determined by the area under the ROC curve was similar in training (0.89) and validation sets (0.87). The training plus validation sets gave an area under the ROC curve of 0.88. Sensitivity, specificity, PPV and NPV for significant fibrosis were determined for various cut-off values between zero and one, in 0.01 increments, in the training set, validation set, training plus validation sets and the singlets set (patients where only one analyte was elevated were excluded).

The full set of data for each sample set evaluated using the Six Variable Model are presented in Tables 11A-D, which includes accuracy, sensitivity, specificity, NPV, and PPV for each possible score at increments of 0.01 for sample sets (Training, Validation, Training+Validation, and Training+Validation excluding singlets). The labels used in Tables 11A-D are the same as those used in Tables 10A-D.

### Example 8

### Results of Six Variable Model: Testing, Validation, and Singlets Sets

Using the constant and coefficients derived above, the diagnostic score cut-off for detecting significant fibrosis was about 0.50. Using a cut-off value of 0.50 for predicting significant fibrosis among the training set of 181 patients resulted in a sensitivity of 81.3% and a specificity of 82.4%. The PPV was 83.9% and NPV was 79.5%. The accuracy in predicting significant fibrosis for this cut-off value was 81.8%. The area under the ROC curve was 0.89.

The predictive model was applied to the validation set of 187 patients, resulting in an area under the curve of 0.87. Using a cut-off value of 0.50 for predicting significant fibrosis among the validation set of 187 patients resulted in a sensitivity of 77.5% and a specificity of 78.6%. The PPV was 76.7% and NPV was 79.4%. The accuracy in predicting significant fibrosis for this cut-off value was 78.1%.

The following table (Table 5) summarizes the results of the accuracy, sensitivity, specificity, PPV, and NPV for a cut-off of 0.50 in the training set, validation set and training plus validation sets.

TABLE 5
Results of Six Variable Model for cut-off of 0.50 for significant fibrosis

| | Samples | | | | | | % correct |
|---|---|---|---|---|---|---|---|
| Patient Set | (n) | Sensitivity | Specificity | Prevalence | PPV | NPV | (accuracy) |
| Training | 181 | 81.3% | 82.4% | 53% | 83.9% | 79.5% | 81.8% |
| Validation | 187 | 77.5% | 78.6% | 47.6% | 76.7% | 79.4% | 78.1% |
| Training + | 368 | 74.2% | 83.7% | 50.3% | 80.5% | 78.1% | 79.9% |
| Validation | | | | | | | |

Analysis using the Six Variable Model was also performed on the testing plus validation set and singlets (Testing+Validation excluding individuals with only one marker elevated). Within the total population (n=368), 268 individuals had only a single elevated analyte. The prevalence of significant fibrosis in the sample excluding singlets was 55%. By removing the singlets and reanalyzing the data, the predictive value of the model improved. The area under the ROC curve for the predictive model was 0.90 for this group.

TABLE 6
Results of Six Variable Model (excluding singlets (n = 268))

| | | | | | % correct |
|---|---|---|---|---|---|
| Cut-off | Sensitivity | Specificity | PPV | NPV | (accuracy) |
| 0.50 | 85.7% | 81% | 84.6% | 82.4% | 83.6% |

The ROC curve for the entire sample set (Testing+Validation) is compared with the Singlet sample set (Testing+Validation excluding singlets) in the graph in FIG. 3.

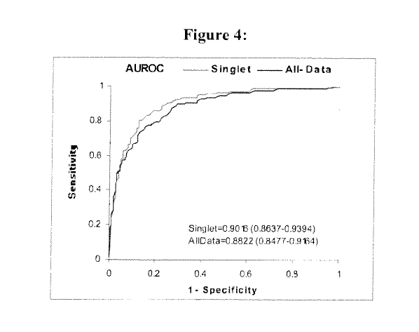

A box plot of the results of the six variable model for stages of fibrosis are found in FIG. 4.

The probability of fibrosis was also evaluated at particular diagnostic scores for the entire population. Table 7 shows the probabilistic likelihoods for each fibrosis stage for individuals with diagnostic scores much higher (e.g., greater than or equal to 0.8) or lower (e.g., less than or equal to 0.3) than the cut-off value. For example, according to the data set, a patient with a diagnostic score of ≦0.1 has a 95.2% probability of being stage F0-1 and a 4.8% probability of being stage F2. Results of this analysis are presented in Table 7. Estimated values of PPV and NPV are also presented in Table 8 for three possible prevalence scenarios using the cut-off value of 0.50 for predicting significant fibrosis.

TABLE 7
Six Variable Model: Probabilities of Each Fibrosis Stage for High
and Low Diagnostic Scores (n = 368)
All-data n = 368

Diagnostic Score (H)

| | ≦0.1 | ≦0.2 | ≦0.3 | ≧0.8 | ≧0.9 |
|---|---|---|---|---|---|
| Population | 42 | 93 | 133 | 106 | 77 |
| F0-1 | 40 (95.2%) | 86 (92.5%) | 115 (86.5%) | 8 (7.5%) | 5 (6.5%) |
| F2-4 | 2 (4.8%) | 7 (7.5%) | 18 (13.5%) | 98 (92.5) | 72 (93.5%) |
| F2 | 2 (4.8%) | 5 (5.4%) | 13 (9.8%) | 14 (13.2) | 6 (7.8%) |
| F3 | 0 (0%) | 1 (1.0%) | 4 (3.0%) | 27 (25.5%) | 18 (23.4%) |
| F4 | 0 (0%) | 1 (1.0%) | 1 (0.7%) | 57 (53.8%) | 48 (62.3%) |

TABLE 8
Estimated values of PPV and NPV

| | Prevalence in | | Prevalence in | | Prevalence in | |
|---|---|---|---|---|---|---|
| | sample 10% | | sample 50% | | sample 70% | |
| | PPV | NPV | PPV | NPV | PPV | NPV |
| Excluding | 33.4% | 98.1% | 81.8% | 70.8% | 91.3% | 70.8% |
| singlets | | | | | | |
| Training | 33.8% | 97.5% | 82.2% | 65.3% | 91.5% | 65.3% |
| Validation | 28.7% | 96.9% | 78.3% | 60% | 89.4% | 60.0% |
| Training + | 31% | 97.2% | 80.2% | 62.6% | 90.4% | 62.6% |
| Validation | | | | | | |

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

### Table 9: Analysis of Sample Sets Using Five Variable Model

TABLE 9A
Analysis of Training set using Five Variable Model; n = 181; Stage 1 (evt) vs. Stage 0 (non-evt).

| | | | | | | | | | | Prevalence | | | | | | |
|---|---|---|---|---|---|---|---|---|---|---|---|---|---|---|---|---|
| | correct | | incorrect | | % | | | | | 53% | | 10.0% | | 70% | | |
| prob | evt | n-e | evt | n-e | accuracy | sensi | spec | f-pos | f-neg | PPV | NPV | PPV | NPV | PPV | NPV | 1-Spec |
| 0 | 96 | 0 | 85 | 0 | 53 | 100 | 0 | 47 | — | 53 | — | 10 | — | 70 | — | 1 |
| 0.01 | 96 | 0 | 85 | 0 | 53 | 100 | 0 | 47 | — | 53 | — | 10 | — | 70 | — | 1 |
| 0.02 | 96 | 0 | 85 | 0 | 53 | 100 | 0 | 47 | — | 53 | — | 10 | — | 70 | — | 1 |
| 0.03 | 96 | 2 | 83 | 0 | 54.1 | 100 | 2.4 | 46.4 | 0 | 53.6 | 100 | 10.2 | 100 | 70.5 | 100 | 0.9765 |
| 0.04 | 95 | 6 | 79 | 1 | 55.8 | 99 | 7.1 | 45.4 | 14.3 | 54.6 | 85.7 | 10.6 | 98.4 | 71.3 | 74.4 | 0.9294 |
| 0.05 | 95 | 7 | 78 | 1 | 56.4 | 99 | 8.2 | 45.1 | 12.5 | 54.9 | 87.5 | 10.7 | 98.6 | 71.6 | 77.2 | 0.9176 |
| 0.06 | 94 | 10 | 75 | 2 | 57.5 | 97.9 | 11.8 | 44.4 | 16.7 | 55.6 | 83.3 | 11 | 98.1 | 72.1 | 70.8 | 0.8824 |
| 0.07 | 94 | 10 | 75 | 2 | 57.5 | 97.9 | 11.8 | 44.4 | 16.7 | 55.6 | 83.3 | 11 | 98.1 | 72.1 | 70.8 | 0.8824 |
| 0.08 | 94 | 12 | 73 | 2 | 58.6 | 97.9 | 14.1 | 43.7 | 14.3 | 56.3 | 85.7 | 11.2 | 98.4 | 72.7 | 74.4 | 0.8588 |
| 0.09 | 94 | 16 | 69 | 2 | 60.8 | 97.9 | 18.8 | 42.3 | 11.1 | 57.7 | 88.9 | 11.8 | 98.8 | 73.8 | 79.5 | 0.8118 |
| 0.1 | 94 | 21 | 64 | 2 | 63.5 | 97.9 | 24.7 | 40.5 | 8.7 | 59.5 | 91.3 | 12.6 | 99.1 | 75.2 | 83.6 | 0.7529 |
| 0.11 | 94 | 22 | 63 | 2 | 64.1 | 97.9 | 25.9 | 40.1 | 8.3 | 59.9 | 91.7 | 12.8 | 99.1 | 75.5 | 84.2 | 0.7412 |
| 0.12 | 94 | 26 | 59 | 2 | 66.3 | 97.9 | 30.6 | 38.6 | 7.1 | 61.4 | 92.9 | 13.6 | 99.2 | 76.7 | 86.3 | 0.6941 |
| 0.13 | 94 | 27 | 58 | 2 | 66.9 | 97.9 | 31.8 | 38.2 | 6.9 | 61.8 | 93.1 | 13.8 | 99.3 | 77 | 86.7 | 0.6824 |
| 0.14 | 94 | 28 | 57 | 2 | 67.4 | 97.9 | 32.9 | 37.7 | 6.7 | 62.3 | 93.3 | 14 | 99.3 | 77.3 | 87.1 | 0.6706 |
| 0.15 | 94 | 32 | 53 | 2 | 69.6 | 97.9 | 37.6 | 36.1 | 5.9 | 63.9 | 94.1 | 14.9 | 99.4 | 78.6 | 88.6 | 0.6235 |
| 0.16 | 93 | 33 | 52 | 3 | 69.6 | 96.9 | 38.8 | 35.9 | 8.3 | 64.1 | 91.7 | 15 | 99.1 | 78.7 | 84.2 | 0.6118 |
| 0.17 | 92 | 34 | 51 | 4 | 69.6 | 95.8 | 40 | 35.7 | 10.5 | 64.3 | 89.5 | 15.1 | 98.9 | 78.8 | 80.4 | 0.6 |
| 0.18 | 92 | 35 | 50 | 4 | 70.2 | 95.8 | 41.2 | 35.2 | 10.3 | 64.8 | 89.7 | 15.3 | 98.9 | 79.2 | 80.9 | 0.5882 |
| 0.19 | 92 | 35 | 50 | 4 | 70.2 | 95.8 | 41.2 | 35.2 | 10.3 | 64.8 | 89.7 | 15.3 | 98.9 | 79.2 | 80.9 | 0.5882 |
| 0.2 | 92 | 36 | 49 | 4 | 70.7 | 95.8 | 42.4 | 34.8 | 10 | 65.2 | 90 | 15.6 | 98.9 | 79.5 | 81.3 | 0.5765 |
| 0.21 | 92 | 37 | 48 | 4 | 71.3 | 95.8 | 43.5 | 34.3 | 9.8 | 65.7 | 90.2 | 15.9 | 98.9 | 79.8 | 81.7 | 0.5647 |
| 0.22 | 92 | 39 | 46 | 4 | 72.4 | 95.8 | 45.9 | 33.3 | 9.3 | 66.7 | 90.7 | 16.4 | 99 | 80.5 | 82.5 | 0.5412 |
| 0.23 | 92 | 40 | 45 | 4 | 72.9 | 95.8 | 47.1 | 32.8 | 9.1 | 67.2 | 90.9 | 16.7 | 99 | 80.9 | 82.9 | 0.5294 |
| 0.24 | 92 | 40 | 45 | 4 | 72.9 | 95.8 | 47.1 | 32.8 | 9.1 | 67.2 | 90.9 | 16.7 | 99 | 80.9 | 82.9 | 0.5294 |
| 0.25 | 92 | 40 | 45 | 4 | 72.9 | 95.8 | 47.1 | 32.8 | 9.1 | 67.2 | 90.9 | 16.7 | 99 | 80.9 | 82.9 | 0.5294 |
| 0.26 | 92 | 43 | 42 | 4 | 74.6 | 95.8 | 50.6 | 31.3 | 8.5 | 68.7 | 91.5 | 17.7 | 99.1 | 81.9 | 83.9 | 0.4941 |
| 0.27 | 91 | 46 | 39 | 5 | 75.7 | 94.8 | 54.1 | 30 | 9.8 | 70 | 90.2 | 18.7 | 98.9 | 82.8 | 81.7 | 0.4588 |
| 0.28 | 91 | 49 | 36 | 5 | 77.3 | 94.8 | 57.6 | 28.3 | 9.3 | 71.7 | 90.7 | 19.9 | 99 | 83.9 | 82.6 | 0.4235 |
| 0.29 | 90 | 50 | 35 | 6 | 77.3 | 93.8 | 58.8 | 28 | 10.7 | 72 | 89.3 | 20.2 | 98.8 | 84.2 | 80.1 | 0.4118 |
| 0.3 | 90 | 52 | 33 | 6 | 78.5 | 93.8 | 61.2 | 26.8 | 10.3 | 73.2 | 89.7 | 21.2 | 98.9 | 84.9 | 80.8 | 0.3882 |
| 0.31 | 90 | 52 | 33 | 6 | 78.5 | 93.8 | 61.2 | 26.8 | 10.3 | 73.2 | 89.7 | 21.2 | 98.9 | 84.9 | 80.8 | 0.3882 |
| 0.32 | 88 | 53 | 32 | 8 | 77.9 | 91.7 | 62.4 | 26.7 | 13.1 | 73.3 | 86.9 | 21.3 | 98.5 | 85 | 76.2 | 0.3765 |
| 0.33 | 86 | 55 | 30 | 10 | 77.9 | 89.6 | 64.7 | 25.9 | 15.4 | 74.1 | 84.6 | 22 | 98.2 | 85.6 | 72.7 | 0.3529 |
| 0.34 | 86 | 55 | 30 | 10 | 77.9 | 89.6 | 64.7 | 25.9 | 15.4 | 74.1 | 84.6 | 22 | 98.2 | 85.6 | 72.7 | 0.3529 |
| 0.35 | 85 | 57 | 28 | 11 | 78.5 | 88.5 | 67.1 | 24.8 | 16.2 | 75.2 | 83.8 | 23 | 98.1 | 86.2 | 71.5 | 0.3294 |
| 0.36 | 84 | 57 | 28 | 12 | 77.9 | 87.5 | 67.1 | 25 | 17.4 | 75 | 82.6 | 22.8 | 98 | 86.1 | 69.7 | 0.3294 |
| 0.37 | 83 | 57 | 28 | 13 | 77.3 | 86.5 | 67.1 | 25.2 | 18.6 | 74.8 | 81.4 | 22.6 | 97.8 | 86 | 68 | 0.3294 |
| 0.38 | 83 | 58 | 27 | 13 | 77.9 | 86.5 | 68.2 | 24.5 | 18.3 | 75.5 | 81.7 | 23.2 | 97.8 | 86.4 | 68.3 | 0.3176 |
| 0.39 | 83 | 58 | 27 | 13 | 77.9 | 86.5 | 68.2 | 24.5 | 18.3 | 75.5 | 81.7 | 23.2 | 97.8 | 86.4 | 68.3 | 0.3176 |
| 0.4 | 83 | 60 | 25 | 13 | 79 | 86.5 | 70.6 | 23.1 | 17.8 | 76.9 | 82.2 | 24.6 | 97.9 | 87.3 | 69.1 | 0.2941 |
| 0.41 | 81 | 62 | 23 | 15 | 79 | 84.4 | 72.9 | 22.1 | 19.5 | 77.9 | 80.5 | 25.7 | 97.7 | 87.9 | 66.7 | 0.2706 |
| 0.42 | 80 | 62 | 23 | 16 | 78.5 | 83.3 | 72.9 | 22.3 | 20.5 | 77.7 | 79.5 | 25.5 | 97.5 | 87.8 | 65.2 | 0.2706 |
| 0.43 | 80 | 62 | 23 | 16 | 78.5 | 83.3 | 72.9 | 22.3 | 20.5 | 77.7 | 79.5 | 25.5 | 97.5 | 87.8 | 65.2 | 0.2706 |
| 0.44 | 78 | 62 | 23 | 18 | 77.3 | 81.3 | 72.9 | 22.8 | 22.5 | 77.2 | 77.5 | 25 | 97.2 | 87.5 | 62.5 | 0.2706 |
| 0.45 | 77 | 63 | 22 | 19 | 77.3 | 80.2 | 74.1 | 22.2 | 23.2 | 77.8 | 76.8 | 25.6 | 97.1 | 87.9 | 61.6 | 0.2588 |
| 0.46 | 77 | 65 | 20 | 19 | 78.5 | 80.2 | 76.5 | 20.6 | 22.6 | 79.4 | 77.4 | 27.5 | 97.2 | 88.8 | 62.3 | 0.2353 |
| 0.47 | 77 | 66 | 19 | 19 | 79 | 80.2 | 77.6 | 19.8 | 22.4 | 80.2 | 77.6 | 28.5 | 97.2 | 89.3 | 62.7 | 0.2235 |
| 0.48 | 74 | 68 | 17 | 22 | 78.5 | 77.1 | 80 | 18.7 | 24.4 | 81.3 | 75.6 | 30 | 96.9 | 90 | 59.9 | 0.2 |
| 0.49 | 73 | 69 | 16 | 23 | 78.5 | 76 | 81.2 | 18 | 25 | 82 | 75 | 31 | 96.8 | 90.4 | 59.2 | 0.1882 |
| 0.5 | 73 | 69 | 16 | 23 | 78.5 | 76 | 81.2 | 18 | 25 | 82 | 75 | 31 | 96.8 | 90.4 | 59.2 | 0.1882 |
| 0.51 | 73 | 70 | 15 | 23 | 79 | 76 | 82.4 | 17 | 24.7 | 83 | 75.3 | 32.4 | 96.9 | 91 | 59.6 | 0.1765 |
| 0.52 | 73 | 70 | 15 | 23 | 79 | 76 | 82.4 | 17 | 24.7 | 83 | 75.3 | 32.4 | 96.9 | 91 | 59.6 | 0.1765 |
| 0.53 | 73 | 71 | 14 | 23 | 79.6 | 76 | 83.5 | 16.1 | 24.5 | 83.9 | 75.5 | 33.9 | 96.9 | 91.5 | 59.9 | 0.1647 |
| 0.54 | 73 | 72 | 13 | 23 | 80.1 | 76 | 84.7 | 15.1 | 24.2 | 84.9 | 75.8 | 35.6 | 97 | 92.1 | 60.2 | 0.1529 |
| 0.55 | 73 | 73 | 12 | 23 | 80.7 | 76 | 85.9 | 14.1 | 24 | 85.9 | 76 | 37.4 | 97 | 92.6 | 60.6 | 0.1412 |
| 0.56 | 72 | 74 | 11 | 24 | 80.7 | 75 | 87.1 | 13.3 | 24.5 | 86.7 | 75.5 | 39.2 | 96.9 | 93.1 | 59.9 | 0.1294 |
| 0.57 | 72 | 74 | 11 | 24 | 80.7 | 75 | 87.1 | 13.3 | 24.5 | 86.7 | 75.5 | 39.2 | 96.9 | 93.1 | 59.9 | 0.1294 |
| 0.58 | 71 | 74 | 11 | 25 | 80.1 | 74 | 87.1 | 13.4 | 25.3 | 86.6 | 74.7 | 38.8 | 96.8 | 93 | 58.9 | 0.1294 |
| 0.59 | 70 | 74 | 11 | 26 | 79.6 | 72.9 | 87.1 | 13.6 | 26 | 86.4 | 74 | 38.5 | 96.7 | 92.9 | 57.9 | 0.1294 |
| 0.6 | 70 | 75 | 10 | 26 | 80.1 | 72.9 | 88.2 | 12.5 | 25.7 | 87.5 | 74.3 | 40.8 | 96.7 | 93.5 | 58.3 | 0.1176 |
| 0.61 | 70 | 76 | 9 | 26 | 80.7 | 72.9 | 89.4 | 11.4 | 25.5 | 88.6 | 74.5 | 43.3 | 96.7 | 94.1 | 58.6 | 0.1059 |
| 0.62 | 70 | 77 | 8 | 26 | 81.2 | 72.9 | 90.6 | 10.3 | 25.2 | 89.7 | 74.8 | 46.3 | 96.8 | 94.8 | 58.9 | 0.0941 |
| 0.63 | 70 | 79 | 6 | 26 | 82.3 | 72.9 | 92.9 | 7.9 | 24.8 | 92.1 | 75.2 | 53.4 | 96.9 | 96 | 59.5 | 0.0706 |
| 0.64 | 68 | 79 | 6 | 28 | 81.2 | 70.8 | 92.9 | 8.1 | 26.2 | 91.9 | 73.8 | 52.7 | 96.6 | 95.9 | 57.7 | 0.0706 |
| 0.65 | 68 | 79 | 6 | 28 | 81.2 | 70.8 | 92.9 | 8.1 | 26.2 | 91.9 | 73.8 | 52.7 | 96.6 | 95.9 | 57.7 | 0.0706 |
| 0.66 | 67 | 79 | 6 | 29 | 80.7 | 69.8 | 92.9 | 8.2 | 26.9 | 91.8 | 73.1 | 52.3 | 96.5 | 95.8 | 56.9 | 0.0706 |
| 0.67 | 67 | 80 | 5 | 29 | 81.2 | 69.8 | 94.1 | 6.9 | 26.6 | 93.1 | 73.4 | 56.9 | 96.6 | 96.5 | 57.2 | 0.0588 |
| 0.68 | 64 | 80 | 5 | 32 | 79.6 | 66.7 | 94.1 | 7.2 | 28.6 | 92.8 | 71.4 | 55.7 | 96.2 | 96.4 | 54.8 | 0.0588 |
| 0.69 | 64 | 80 | 5 | 32 | 79.6 | 66.7 | 94.1 | 7.2 | 28.6 | 92.8 | 71.4 | 55.7 | 96.2 | 96.4 | 54.8 | 0.0588 |
| 0.7 | 63 | 80 | 5 | 33 | 79 | 65.6 | 94.1 | 7.4 | 29.2 | 92.6 | 70.8 | 55.3 | 96.1 | 96.3 | 54 | 0.0588 |
| 0.71 | 62 | 80 | 5 | 34 | 78.5 | 64.6 | 94.1 | 7.5 | 29.8 | 92.5 | 70.2 | 55 | 96 | 96.2 | 53.2 | 0.0588 |
| 0.72 | 61 | 80 | 5 | 35 | 77.9 | 63.5 | 94.1 | 7.6 | 30.4 | 92.4 | 69.6 | 54.6 | 95.9 | 96.2 | 52.5 | 0.0588 |
| 0.73 | 60 | 80 | 5 | 36 | 77.3 | 62.5 | 94.1 | 7.7 | 31 | 92.3 | 69 | 54.1 | 95.8 | 96.1 | 51.8 | 0.0588 |
| 0.74 | 57 | 80 | 5 | 39 | 75.7 | 59.4 | 94.1 | 8.1 | 32.8 | 91.9 | 67.2 | 52.9 | 95.4 | 95.9 | 49.8 | 0.0588 |
| 0.75 | 55 | 80 | 5 | 41 | 74.6 | 57.3 | 94.1 | 8.3 | 33.9 | 91.7 | 66.1 | 52 | 95.2 | 95.8 | 48.6 | 0.0588 |
| 0.76 | 53 | 80 | 5 | 43 | 73.5 | 55.2 | 94.1 | 8.6 | 35 | 91.4 | 65 | 51 | 95 | 95.6 | 47.4 | 0.0588 |
| 0.77 | 51 | 81 | 4 | 45 | 72.9 | 53.1 | 95.3 | 7.3 | 35.7 | 92.7 | 64.3 | 55.6 | 94.8 | 96.3 | 46.6 | 0.0471 |
| 0.78 | 49 | 81 | 4 | 47 | 71.8 | 51 | 95.3 | 7.5 | 36.7 | 92.5 | 63.3 | 54.7 | 94.6 | 96.2 | 45.5 | 0.0471 |
| 0.79 | 48 | 81 | 4 | 48 | 71.3 | 50 | 95.3 | 7.7 | 37.2 | 92.3 | 62.8 | 54.1 | 94.5 | 96.1 | 45 | 0.0471 |
| 0.8 | 47 | 81 | 4 | 49 | 70.7 | 49 | 95.3 | 7.8 | 37.7 | 92.2 | 62.3 | 53.6 | 94.4 | 96 | 44.4 | 0.0471 |
| 0.81 | 47 | 81 | 4 | 49 | 70.7 | 49 | 95.3 | 7.8 | 37.7 | 92.2 | 62.3 | 53.6 | 94.4 | 96 | 44.4 | 0.0471 |
| 0.82 | 47 | 81 | 4 | 49 | 70.7 | 49 | 95.3 | 7.8 | 37.7 | 92.2 | 62.3 | 53.6 | 94.4 | 96 | 44.4 | 0.0471 |
| 0.83 | 47 | 82 | 3 | 49 | 71.3 | 49 | 96.5 | 6 | 37.4 | 94 | 62.6 | 60.6 | 94.4 | 97 | 44.8 | 0.0353 |
| 0.84 | 47 | 83 | 2 | 49 | 71.8 | 49 | 97.6 | 4.1 | 37.1 | 95.9 | 62.9 | 69.8 | 94.5 | 98 | 45.1 | 0.0235 |
| 0.85 | 46 | 83 | 2 | 50 | 71.3 | 47.9 | 97.6 | 4.2 | 37.6 | 95.8 | 62.4 | 69.4 | 94.4 | 97.9 | 44.6 | 0.0235 |
| 0.86 | 43 | 83 | 2 | 53 | 69.6 | 44.8 | 97.6 | 4.4 | 39 | 95.6 | 61 | 67.9 | 94.1 | 97.8 | 43.1 | 0.0235 |
| 0.87 | 42 | 83 | 2 | 54 | 69.1 | 43.8 | 97.6 | 4.5 | 39.4 | 95.5 | 60.6 | 67.4 | 94 | 97.7 | 42.7 | 0.0235 |
| 0.88 | 40 | 83 | 2 | 56 | 68 | 41.7 | 97.6 | 4.8 | 40.3 | 95.2 | 59.7 | 66.3 | 93.8 | 97.6 | 41.8 | 0.0235 |
| 0.89 | 38 | 83 | 2 | 58 | 66.9 | 39.6 | 97.6 | 5 | 41.1 | 95 | 58.9 | 65.1 | 93.6 | 97.5 | 40.9 | 0.0235 |
| 0.9 | 37 | 83 | 2 | 59 | 66.3 | 38.5 | 97.6 | 5.1 | 41.5 | 94.9 | 58.5 | 64.5 | 93.5 | 97.5 | 40.5 | 0.0235 |
| 0.91 | 37 | 83 | 2 | 59 | 66.3 | 38.5 | 97.6 | 5.1 | 41.5 | 94.9 | 58.5 | 64.5 | 93.5 | 97.5 | 40.5 | 0.0235 |
| 0.92 | 36 | 83 | 2 | 60 | 65.7 | 37.5 | 97.6 | 5.3 | 42 | 94.7 | 58 | 63.9 | 93.4 | 97.4 | 40.1 | 0.0235 |
| 0.93 | 35 | 83 | 2 | 61 | 65.2 | 36.5 | 97.6 | 5.4 | 42.4 | 94.6 | 57.6 | 63.3 | 93.3 | 97.3 | 39.7 | 0.0235 |
| 0.94 | 32 | 84 | 1 | 64 | 64.1 | 33.3 | 98.8 | 3 | 43.2 | 97 | 56.8 | 75.9 | 93 | 98.5 | 38.8 | 0.0118 |
| 0.95 | 30 | 84 | 1 | 66 | 63 | 31.3 | 98.8 | 3.2 | 44 | 96.8 | 56 | 74.7 | 92.8 | 98.4 | 38.1 | 0.0118 |
| 0.96 | 26 | 84 | 1 | 70 | 60.8 | 27.1 | 98.8 | 3.7 | 45.5 | 96.3 | 54.5 | 71.9 | 92.4 | 98.2 | 36.7 | 0.0118 |
| 0.97 | 21 | 85 | 0 | 75 | 58.6 | 21.9 | 100 | 0 | 46.9 | 100 | 53.1 | 100 | 92 | 100 | 35.4 | 0 |
| 0.98 | 18 | 85 | 0 | 78 | 56.9 | 18.8 | 100 | 0 | 47.9 | 100 | 52.1 | 100 | 91.7 | 100 | 34.5 | 0 |
| 0.99 | 14 | 85 | 0 | 82 | 54.7 | 14.6 | 100 | 0 | 49.1 | 100 | 50.9 | 100 | 91.3 | 100 | 33.4 | 0 |
| 1 | 0 | 85 | 0 | 96 | 47 | 0 | 100 | — | 53 | — | 47 | — | 90 | — | 30 | 0 |

TABLE 9B
Analysis of Validation (testing) set using Five Variable Model; n = 187; Stage 1 (evt) vs. Stage 0 (non-evt).

| | | | | | | | | | | Prevalence | | | | | | |
|---|---|---|---|---|---|---|---|---|---|---|---|---|---|---|---|---|
| | correct | | incorrect | | % | | | | | 47.6% | | 10.0% | | 70% | | |
| prob | evt | n-e | evt | n-e | accuracy | sensi | spec | f-pos | f-neg | PPV | NPV | PPV | NPV | PPV | NPV | 1-Spec |
| 0 | 89 | 0 | 98 | 0 | 47.6 | 100 | 0 | 52.4 | — | 47.6 | — | 10 | — | 70 | — | 1 |
| 0.01 | 89 | 0 | 98 | 0 | 47.6 | 100 | 0 | 52.4 | — | 47.6 | — | 10 | — | 70 | — | 1 |
| 0.02 | 89 | 1 | 97 | 0 | 48.1 | 100 | 1 | 52.2 | 0 | 47.8 | 100 | 10.1 | 100 | 70.2 | 100 | 0.9898 |
| 0.03 | 89 | 1 | 97 | 0 | 48.1 | 100 | 1 | 52.2 | 0 | 47.8 | 100 | 10.1 | 100 | 70.2 | 100 | 0.9898 |
| 0.04 | 89 | 4 | 94 | 0 | 49.7 | 100 | 4.1 | 51.4 | 0 | 48.6 | 100 | 10.4 | 100 | 70.9 | 100 | 0.9592 |
| 0.05 | 89 | 5 | 93 | 0 | 50.3 | 100 | 5.1 | 51.1 | 0 | 48.9 | 100 | 10.5 | 100 | 71.1 | 100 | 0.949 |
| 0.06 | 89 | 11 | 87 | 0 | 53.5 | 100 | 11.2 | 49.4 | 0 | 50.6 | 100 | 11.1 | 100 | 72.4 | 100 | 0.8878 |
| 0.07 | 89 | 12 | 86 | 0 | 54 | 100 | 12.2 | 49.1 | 0 | 50.9 | 100 | 11.2 | 100 | 72.7 | 100 | 0.8776 |
| 0.08 | 89 | 14 | 84 | 0 | 55.1 | 100 | 14.3 | 48.6 | 0 | 51.4 | 100 | 11.5 | 100 | 73.1 | 100 | 0.8571 |
| 0.09 | 89 | 17 | 81 | 0 | 56.7 | 100 | 17.3 | 47.6 | 0 | 52.4 | 100 | 11.9 | 100 | 73.8 | 100 | 0.8265 |
| 0.1 | 88 | 20 | 78 | 1 | 57.8 | 98.9 | 20.4 | 47 | 4.8 | 53 | 95.2 | 12.1 | 99.4 | 74.4 | 88.6 | 0.7959 |
| 0.11 | 88 | 21 | 77 | 1 | 58.3 | 98.9 | 21.4 | 46.7 | 4.5 | 53.3 | 95.5 | 12.3 | 99.4 | 74.6 | 89.1 | 0.7857 |
| 0.12 | 88 | 27 | 71 | 1 | 61.5 | 98.9 | 27.6 | 44.7 | 3.6 | 55.3 | 96.4 | 13.2 | 99.5 | 76.1 | 91.3 | 0.7245 |
| 0.13 | 88 | 30 | 68 | 1 | 63.1 | 98.9 | 30.6 | 43.6 | 3.2 | 56.4 | 96.8 | 13.7 | 99.6 | 76.9 | 92.1 | 0.6939 |
| 0.14 | 88 | 33 | 65 | 1 | 64.7 | 98.9 | 33.7 | 42.5 | 2.9 | 57.5 | 97.1 | 14.2 | 99.6 | 77.7 | 92.8 | 0.6633 |
| 0.15 | 88 | 37 | 61 | 1 | 66.8 | 98.9 | 37.8 | 40.9 | 2.6 | 59.1 | 97.4 | 15 | 99.7 | 78.8 | 93.5 | 0.6224 |
| 0.16 | 88 | 37 | 61 | 1 | 66.8 | 98.9 | 37.8 | 40.9 | 2.6 | 59.1 | 97.4 | 15 | 99.7 | 78.8 | 93.5 | 0.6224 |
| 0.17 | 88 | 39 | 59 | 1 | 67.9 | 98.9 | 39.8 | 40.1 | 2.5 | 59.9 | 97.5 | 15.4 | 99.7 | 79.3 | 93.8 | 0.602 |
| 0.18 | 87 | 42 | 56 | 2 | 69 | 97.8 | 42.9 | 39.2 | 4.5 | 60.8 | 95.5 | 16 | 99.4 | 80 | 89.1 | 0.5714 |
| 0.19 | 86 | 45 | 53 | 3 | 70.1 | 96.6 | 45.9 | 38.1 | 6.3 | 61.9 | 93.8 | 16.6 | 99.2 | 80.7 | 85.4 | 0.5408 |
| 0.2 | 86 | 47 | 51 | 3 | 71.1 | 96.6 | 48 | 37.2 | 6 | 62.8 | 94 | 17.1 | 99.2 | 81.2 | 85.9 | 0.5204 |
| 0.21 | 86 | 50 | 48 | 3 | 72.7 | 96.6 | 51 | 35.8 | 5.7 | 64.2 | 94.3 | 18 | 99.3 | 82.2 | 86.6 | 0.4898 |
| 0.22 | 86 | 51 | 47 | 3 | 73.3 | 96.6 | 52 | 35.3 | 5.6 | 64.7 | 94.4 | 18.3 | 99.3 | 82.5 | 86.9 | 0.4796 |
| 0.23 | 86 | 51 | 47 | 3 | 73.3 | 96.6 | 52 | 35.3 | 5.6 | 64.7 | 94.4 | 18.3 | 99.3 | 82.5 | 86.9 | 0.4796 |
| 0.24 | 84 | 53 | 45 | 5 | 73.3 | 94.4 | 54.1 | 34.9 | 8.6 | 65.1 | 91.4 | 18.6 | 98.9 | 82.7 | 80.5 | 0.4592 |
| 0.25 | 82 | 53 | 45 | 7 | 72.2 | 92.1 | 54.1 | 35.4 | 11.7 | 64.6 | 88.3 | 18.2 | 98.4 | 82.4 | 74.7 | 0.4592 |
| 0.26 | 82 | 56 | 42 | 7 | 73.8 | 92.1 | 57.1 | 33.9 | 11.1 | 66.1 | 88.9 | 19.3 | 98.5 | 83.4 | 75.7 | 0.4286 |
| 0.27 | 82 | 59 | 39 | 7 | 75.4 | 92.1 | 60.2 | 32.2 | 10.6 | 67.8 | 89.4 | 20.5 | 98.6 | 84.4 | 76.6 | 0.398 |
| 0.28 | 82 | 60 | 38 | 7 | 75.9 | 92.1 | 61.2 | 31.7 | 10.4 | 68.3 | 89.6 | 20.9 | 98.6 | 84.7 | 76.9 | 0.3878 |
| 0.29 | 82 | 63 | 35 | 7 | 77.5 | 92.1 | 64.3 | 29.9 | 10 | 70.1 | 90 | 22.3 | 98.7 | 85.8 | 77.8 | 0.3571 |
| 0.3 | 81 | 64 | 34 | 8 | 77.5 | 91 | 65.3 | 29.6 | 11.1 | 70.4 | 88.9 | 22.6 | 98.5 | 86 | 75 | 0.3469 |
| 0.31 | 81 | 64 | 34 | 8 | 77.5 | 91 | 65.3 | 29.6 | 11.1 | 70.4 | 88.9 | 22.6 | 98.5 | 86 | 75.7 | 0.3469 |
| 0.32 | 80 | 64 | 34 | 9 | 77 | 89.9 | 65.3 | 29.8 | 12.3 | 70.2 | 87.7 | 22.4 | 98.3 | 85.8 | 73.5 | 0.3469 |
| 0.33 | 79 | 64 | 34 | 10 | 76.5 | 88.8 | 65.3 | 30.1 | 13.5 | 69.9 | 86.5 | 22.1 | 98.1 | 85.7 | 71.4 | 0.3469 |
| 0.34 | 79 | 64 | 34 | 10 | 76.5 | 88.8 | 65.3 | 30.1 | 13.5 | 69.9 | 86.5 | 22.1 | 98.1 | 85.7 | 71.4 | 0.3469 |
| 0.35 | 79 | 64 | 34 | 10 | 76.5 | 88.8 | 65.3 | 30.1 | 13.5 | 69.9 | 86.5 | 22.1 | 98.1 | 85.7 | 71.4 | 0.3469 |
| 0.36 | 78 | 67 | 31 | 11 | 77.5 | 87.6 | 68.4 | 28.4 | 14.1 | 71.6 | 85.9 | 23.5 | 98 | 86.6 | 70.3 | 0.3163 |
| 0.37 | 78 | 69 | 29 | 11 | 78.6 | 87.6 | 70.4 | 27.1 | 13.8 | 72.9 | 86.3 | 24.8 | 98.1 | 87.4 | 70.9 | 0.2959 |
| 0.38 | 76 | 69 | 29 | 13 | 77.5 | 85.4 | 70.4 | 27.6 | 15.9 | 72.4 | 84.1 | 24.3 | 97.7 | 87.1 | 67.4 | 0.2959 |
| 0.39 | 76 | 71 | 27 | 13 | 78.6 | 85.4 | 72.4 | 26.2 | 15.5 | 73.8 | 84.5 | 25.6 | 97.8 | 87.9 | 68 | 0.2755 |
| 0.4 | 76 | 71 | 27 | 13 | 78.6 | 85.4 | 72.4 | 26.2 | 15.5 | 73.8 | 84.5 | 25.6 | 97.8 | 87.9 | 68 | 0.2755 |
| 0.41 | 75 | 71 | 27 | 14 | 78.1 | 84.3 | 72.4 | 26.5 | 16.5 | 73.5 | 83.5 | 25.4 | 97.6 | 87.7 | 66.4 | 0.2755 |
| 0.42 | 75 | 71 | 27 | 14 | 78.1 | 84.3 | 72.4 | 26.5 | 16.5 | 73.5 | 83.5 | 25.4 | 97.6 | 87.7 | 66.4 | 0.2755 |
| 0.43 | 75 | 74 | 24 | 14 | 79.7 | 84.3 | 75.5 | 24.5 | 15.9 | 75.8 | 84.1 | 27.7 | 97.7 | 88.9 | 67.3 | 0.2449 |
| 0.44 | 75 | 75 | 23 | 14 | 80.2 | 84.3 | 76.5 | 23.5 | 15.7 | 76.5 | 84.3 | 28.5 | 97.8 | 89.3 | 67.6 | 0.2347 |
| 0.45 | 74 | 75 | 23 | 15 | 79.7 | 83.1 | 76.5 | 23.7 | 16.7 | 76.3 | 83.3 | 28.2 | 97.6 | 89.2 | 66.1 | 0.2347 |
| 0.46 | 74 | 77 | 21 | 15 | 80.7 | 83.1 | 78.6 | 22.1 | 16.3 | 77.9 | 83.7 | 30.1 | 97.7 | 90.1 | 66.6 | 0.2143 |
| 0.47 | 74 | 77 | 21 | 15 | 80.7 | 83.1 | 78.6 | 22.1 | 16.3 | 77.9 | 83.7 | 30.1 | 97.7 | 90.1 | 66.6 | 0.2143 |
| 0.48 | 73 | 78 | 20 | 16 | 80.7 | 82 | 79.6 | 21.5 | 17 | 78.5 | 83 | 30.9 | 97.6 | 90.4 | 65.5 | 0.2041 |
| 0.49 | 73 | 80 | 18 | 16 | 81.8 | 82 | 81.6 | 19.8 | 16.7 | 80.2 | 83.3 | 33.2 | 97.6 | 91.2 | 66.1 | 0.1837 |
| 0.5 | 72 | 80 | 18 | 17 | 81.3 | 80.9 | 81.6 | 20 | 17.5 | 80 | 82.5 | 32.9 | 97.5 | 91.1 | 64.7 | 0.1837 |
| 0.51 | 71 | 82 | 16 | 18 | 81.8 | 79.8 | 83.7 | 18.4 | 18 | 81.6 | 82 | 35.2 | 97.4 | 91.9 | 63.9 | 0.1633 |
| 0.52 | 71 | 82 | 16 | 18 | 81.8 | 79.8 | 83.7 | 18.4 | 18 | 81.6 | 82 | 35.2 | 97.4 | 91.9 | 63.9 | 0.1633 |
| 0.53 | 69 | 82 | 16 | 20 | 80.7 | 77.5 | 83.7 | 18.8 | 19.6 | 81.2 | 80.4 | 34.5 | 97.1 | 91.7 | 61.5 | 0.1633 |
| 0.54 | 67 | 84 | 14 | 22 | 80.7 | 75.3 | 85.7 | 17.3 | 20.8 | 82.7 | 79.2 | 36.9 | 96.9 | 92.5 | 59.8 | 0.1429 |
| 0.55 | 66 | 84 | 14 | 23 | 80.2 | 74.2 | 85.7 | 17.5 | 21.5 | 82.5 | 78.5 | 36.6 | 96.8 | 92.4 | 58.7 | 0.1429 |
| 0.56 | 65 | 84 | 14 | 24 | 79.7 | 73 | 85.7 | 17.7 | 22.2 | 82.3 | 77.8 | 36.2 | 96.6 | 92.3 | 57.7 | 0.1429 |
| 0.57 | 64 | 84 | 14 | 25 | 79.1 | 71.9 | 85.7 | 17.9 | 22.9 | 82.1 | 77.1 | 35.9 | 96.5 | 92.2 | 56.7 | 0.1429 |
| 0.58 | 64 | 84 | 14 | 25 | 79.1 | 71.9 | 85.7 | 17.9 | 22.9 | 82.1 | 77.1 | 35.9 | 96.5 | 92.2 | 56.7 | 0.1429 |
| 0.59 | 64 | 85 | 13 | 25 | 79.7 | 71.9 | 86.7 | 16.9 | 22.7 | 83.1 | 77.3 | 37.6 | 96.5 | 92.7 | 57 | 0.1327 |
| 0.6 | 64 | 85 | 13 | 25 | 79.7 | 71.9 | 86.7 | 16.9 | 22.7 | 83.1 | 77.3 | 37.6 | 96.5 | 92.7 | 57 | 0.1327 |
| 0.61 | 61 | 85 | 13 | 28 | 78.1 | 68.5 | 86.7 | 17.6 | 24.8 | 82.4 | 75.2 | 36.5 | 96.1 | 92.3 | 54.2 | 0.1327 |
| 0.62 | 60 | 85 | 13 | 29 | 77.5 | 67.4 | 86.7 | 17.8 | 25.4 | 82.2 | 74.6 | 36.1 | 96 | 92.2 | 53.3 | 0.1327 |
| 0.63 | 58 | 86 | 12 | 31 | 77 | 65.2 | 87.8 | 17.1 | 26.5 | 82.9 | 73.5 | 37.2 | 95.8 | 92.5 | 51.9 | 0.1224 |
| 0.64 | 58 | 88 | 10 | 31 | 78.1 | 65.2 | 89.8 | 14.7 | 26.1 | 85.3 | 73.9 | 41.5 | 95.9 | 93.7 | 52.5 | 0.102 |
| 0.65 | 58 | 90 | 8 | 31 | 79.1 | 65.2 | 91.8 | 12.1 | 25.6 | 87.9 | 74.4 | 47 | 96 | 94.9 | 53.1 | 0.0816 |
| 0.66 | 56 | 90 | 8 | 33 | 78.1 | 62.9 | 91.8 | 12.5 | 26.8 | 87.5 | 73.2 | 46.1 | 95.7 | 94.7 | 51.5 | 0.0816 |
| 0.67 | 56 | 90 | 8 | 33 | 78.1 | 62.9 | 91.8 | 12.5 | 26.8 | 87.5 | 73.2 | 46.1 | 95.7 | 94.7 | 51.5 | 0.0816 |
| 0.68 | 55 | 90 | 8 | 34 | 77.5 | 61.8 | 91.8 | 12.7 | 27.4 | 87.3 | 72.6 | 45.7 | 95.6 | 94.6 | 50.7 | 0.0816 |
| 0.69 | 53 | 90 | 8 | 36 | 76.5 | 59.6 | 91.8 | 13.1 | 28.6 | 86.9 | 71.4 | 44.8 | 95.3 | 94.5 | 49.3 | 0.0816 |
| 0.7 | 53 | 90 | 8 | 36 | 76.5 | 59.6 | 91.8 | 13.1 | 28.6 | 86.9 | 71.4 | 44.8 | 95.3 | 94.5 | 49.3 | 0.0816 |
| 0.71 | 53 | 90 | 8 | 36 | 76.5 | 59.6 | 91.8 | 13.1 | 28.6 | 86.9 | 71.4 | 44.8 | 95.3 | 94.5 | 49.3 | 0.0816 |
| 0.72 | 52 | 91 | 7 | 37 | 76.5 | 58.4 | 92.9 | 11.9 | 28.9 | 88.1 | 71.1 | 47.6 | 95.3 | 95 | 48.9 | 0.0714 |
| 0.73 | 52 | 91 | 7 | 37 | 76.5 | 58.4 | 92.9 | 11.9 | 28.9 | 88.1 | 71.1 | 47.6 | 95.3 | 95 | 48.9 | 0.0714 |
| 0.74 | 51 | 91 | 7 | 38 | 75.9 | 57.3 | 92.9 | 12.1 | 29.5 | 87.9 | 70.5 | 47.1 | 95.1 | 94.9 | 48.2 | 0.0714 |
| 0.75 | 50 | 92 | 6 | 39 | 75.9 | 56.2 | 93.9 | 10.7 | 29.8 | 89.3 | 70.2 | 50.5 | 95.1 | 95.5 | 47.9 | 0.0612 |
| 0.76 | 47 | 92 | 6 | 42 | 74.3 | 52.8 | 93.9 | 11.3 | 31.3 | 88.7 | 68.7 | 48.9 | 94.7 | 95.3 | 46 | 0.0612 |
| 0.77 | 46 | 92 | 6 | 43 | 73.8 | 51.7 | 93.9 | 11.5 | 31.9 | 88.5 | 68.1 | 48.4 | 94.6 | 95.2 | 45.4 | 0.0612 |
| 0.78 | 46 | 92 | 6 | 43 | 73.8 | 51.7 | 93.9 | 11.5 | 31.9 | 88.5 | 68.1 | 48.4 | 94.6 | 95.2 | 45.4 | 0.0612 |
| 0.79 | 46 | 92 | 6 | 43 | 73.8 | 51.7 | 93.9 | 11.5 | 31.9 | 88.5 | 68.1 | 48.4 | 94.6 | 95.2 | 45.4 | 0.0612 |
| 0.8 | 45 | 94 | 4 | 44 | 74.3 | 50.6 | 95.9 | 8.2 | 31.9 | 91.8 | 68.1 | 57.9 | 94.6 | 96.7 | 45.4 | 0.0408 |
| 0.81 | 44 | 94 | 4 | 45 | 73.8 | 49.4 | 95.9 | 8.3 | 32.4 | 91.7 | 67.6 | 57.4 | 94.5 | 96.6 | 44.8 | 0.0408 |
| 0.82 | 42 | 94 | 4 | 47 | 72.7 | 47.2 | 95.9 | 8.7 | 33.3 | 91.3 | 66.7 | 56.2 | 94.2 | 96.4 | 43.8 | 0.0408 |
| 0.83 | 40 | 94 | 4 | 49 | 71.7 | 44.9 | 95.9 | 9.1 | 34.3 | 90.9 | 65.7 | 55 | 94 | 96.3 | 42.7 | 0.0408 |
| 0.84 | 39 | 94 | 4 | 50 | 71.1 | 43.8 | 95.9 | 9.3 | 34.7 | 90.7 | 65.3 | 54.4 | 93.9 | 96.2 | 42.3 | 0.0408 |
| 0.85 | 37 | 95 | 3 | 52 | 70.6 | 41.6 | 96.9 | 7.5 | 35.4 | 92.5 | 64.6 | 60.1 | 93.7 | 96.9 | 41.6 | 0.0306 |
| 0.86 | 36 | 95 | 3 | 53 | 70.1 | 40.4 | 96.9 | 7.7 | 35.8 | 92.3 | 64.2 | 59.5 | 93.6 | 96.9 | 41.1 | 0.0306 |
| 0.87 | 32 | 95 | 3 | 57 | 67.9 | 36 | 96.9 | 8.6 | 37.5 | 91.4 | 62.5 | 56.6 | 93.2 | 96.5 | 39.3 | 0.0306 |
| 0.88 | 32 | 95 | 3 | 57 | 67.9 | 36 | 96.9 | 8.6 | 37.5 | 91.4 | 62.5 | 56.6 | 93.2 | 96.5 | 39.3 | 0.0306 |
| 0.89 | 30 | 96 | 2 | 59 | 67.4 | 33.7 | 98 | 6.3 | 38.1 | 93.8 | 61.9 | 64.7 | 93 | 97.5 | 38.8 | 0.0204 |
| 0.9 | 30 | 97 | 1 | 59 | 67.9 | 33.7 | 99 | 3.2 | 37.8 | 96.8 | 62.2 | 78.6 | 93.1 | 98.7 | 39 | 0.0102 |
| 0.91 | 30 | 97 | 1 | 59 | 67.9 | 33.7 | 99 | 3.2 | 37.8 | 96.8 | 62.2 | 78.6 | 93.1 | 98.7 | 39 | 0.0102 |
| 0.92 | 28 | 97 | 1 | 61 | 66.8 | 31.5 | 99 | 3.4 | 38.6 | 96.6 | 61.4 | 77.4 | 92.9 | 98.6 | 38.2 | 0.0102 |
| 0.93 | 27 | 97 | 1 | 62 | 66.3 | 30.3 | 99 | 3.6 | 39 | 96.4 | 61 | 76.8 | 92.7 | 98.6 | 37.8 | 0.0102 |
| 0.94 | 25 | 97 | 1 | 64 | 65.2 | 28.1 | 99 | 3.8 | 39.8 | 96.2 | 60.2 | 75.4 | 92.5 | 98.5 | 37.1 | 0.0102 |
| 0.95 | 23 | 97 | 1 | 66 | 64.2 | 25.8 | 99 | 4.2 | 40.5 | 95.8 | 59.5 | 73.8 | 92.3 | 98.3 | 36.4 | 0.0102 |
| 0.96 | 22 | 97 | 1 | 67 | 63.6 | 24.7 | 99 | 4.3 | 40.9 | 95.7 | 59.1 | 72.9 | 92.2 | 98.3 | 36 | 0.0102 |
| 0.97 | 21 | 98 | 0 | 68 | 63.6 | 23.6 | 100 | 0 | 41 | 100 | 59 | 100 | 92.2 | 100 | 35.9 | 0 |
| 0.98 | 19 | 98 | 0 | 70 | 62.6 | 21.3 | 100 | 0 | 41.7 | 100 | 58.3 | 100 | 92 | 100 | 35.3 | 0 |
| 0.99 | 15 | 98 | 0 | 74 | 60.4 | 16.9 | 100 | 0 | 43 | 100 | 57 | 100 | 91.5 | 100 | 34 | 0 |
| 1 | 0 | 98 | 0 | 89 | 52.4 | 0 | 100 | — | 47.6 | — | 52.4 | — | 90 | — | 30 | 0 |

TABLE 9C
Analysis of Training plus Validation sets using Five Variable Model; n = 368; Stage 1 (evt) vs. Stage 0 (non-evt).

| | | | | | | | | | | Prevalence | | | | | | |
|---|---|---|---|---|---|---|---|---|---|---|---|---|---|---|---|---|
| | correct | | incorrect | | % | | | | | 50.3% | | 10.0% | | 70% | | |
| prob | evt | n-e | evt | n-e | accuracy | sensi | spec | f-pos | f-neg | PPV | NPV | PPV | NPV | PPV | NPV | 1-Spec |
| 0 | 185 | 0 | 183 | 0 | 50.3 | 100 | 0 | 49.7 | — | 50.3 | — | 10 | — | 70 | — | 1 |
| 0.01 | 185 | 0 | 183 | 0 | 50.3 | 100 | 0 | 49.7 | — | 50.3 | — | 10 | — | 70 | — | 1 |
| 0.02 | 185 | 1 | 182 | 0 | 50.5 | 100 | 0.5 | 49.6 | 0 | 50.4 | 100 | 10 | 100 | 70.1 | 100 | 0.9945 |
| 0.03 | 185 | 3 | 180 | 0 | 51.1 | 100 | 1.6 | 49.3 | 0 | 50.7 | 100 | 10.1 | 100 | 70.3 | 100 | 0.9836 |
| 0.04 | 184 | 10 | 173 | 1 | 52.7 | 99.5 | 5.5 | 48.5 | 9.1 | 51.5 | 90.9 | 10.5 | 98.9 | 71.1 | 81.2 | 0.9454 |
| 0.05 | 184 | 12 | 171 | 1 | 53.3 | 99.5 | 6.6 | 48.2 | 7.7 | 51.8 | 92.3 | 10.6 | 99.1 | 71.3 | 83.9 | 0.9344 |
| 0.06 | 183 | 21 | 162 | 2 | 55.4 | 98.9 | 11.5 | 47 | 8.7 | 53 | 91.3 | 11 | 99 | 72.3 | 82 | 0.8852 |
| 0.07 | 183 | 22 | 161 | 2 | 55.7 | 98.9 | 12 | 46.8 | 8.3 | 53.2 | 91.7 | 11.1 | 99 | 72.4 | 82.7 | 0.8798 |
| 0.08 | 183 | 26 | 157 | 2 | 56.8 | 98.9 | 14.2 | 46.2 | 7.1 | 53.8 | 92.9 | 11.4 | 99.2 | 72.9 | 84.9 | 0.8579 |
| 0.09 | 183 | 33 | 150 | 2 | 58.7 | 98.9 | 18 | 45 | 5.7 | 55 | 94.3 | 11.8 | 99.3 | 73.8 | 87.7 | 0.8197 |
| 0.1 | 182 | 41 | 142 | 3 | 60.6 | 98.4 | 22.4 | 43.8 | 6.8 | 56.2 | 93.2 | 12.3 | 99.2 | 74.7 | 85.6 | 0.776 |
| 0.11 | 182 | 43 | 140 | 3 | 61.1 | 98.4 | 23.5 | 43.5 | 6.5 | 56.5 | 93.5 | 12.5 | 99.2 | 75 | 86.1 | 0.765 |
| 0.12 | 182 | 53 | 130 | 3 | 63.9 | 98.4 | 29 | 41.7 | 5.4 | 58.3 | 94.6 | 13.3 | 99.4 | 76.4 | 88.4 | 0.7104 |
| 0.13 | 182 | 57 | 126 | 3 | 64.9 | 98.4 | 31.1 | 40.9 | 5 | 59.1 | 95 | 13.7 | 99.4 | 76.9 | 89.2 | 0.6885 |
| 0.14 | 182 | 61 | 122 | 3 | 66 | 98.4 | 33.3 | 40.1 | 4.7 | 59.9 | 95.3 | 14.1 | 99.5 | 77.5 | 89.8 | 0.6667 |
| 0.15 | 182 | 69 | 114 | 3 | 68.2 | 98.4 | 37.7 | 38.5 | 4.2 | 61.5 | 95.8 | 14.9 | 99.5 | 78.7 | 90.9 | 0.623 |
| 0.16 | 181 | 70 | 113 | 4 | 68.2 | 97.8 | 38.3 | 38.4 | 5.4 | 61.6 | 94.6 | 15 | 99.4 | 78.7 | 88.3 | 0.6175 |
| 0.17 | 180 | 73 | 110 | 5 | 68.8 | 97.3 | 39.9 | 37.9 | 6.4 | 62.1 | 93.6 | 15.2 | 99.3 | 79.1 | 86.3 | 0.6011 |
| 0.18 | 179 | 77 | 106 | 6 | 69.6 | 96.8 | 42.1 | 37.2 | 7.2 | 62.8 | 92.8 | 15.7 | 99.2 | 79.6 | 84.8 | 0.5792 |
| 0.19 | 178 | 80 | 103 | 7 | 70.1 | 96.2 | 43.7 | 36.7 | 8 | 63.3 | 92 | 16 | 99 | 80 | 83.2 | 0.5628 |
| 0.2 | 178 | 84 | 99 | 7 | 71.2 | 96.2 | 45.9 | 35.7 | 7.7 | 64.3 | 92.3 | 16.5 | 99.1 | 80.6 | 83.9 | 0.541 |
| 0.21 | 178 | 89 | 94 | 7 | 72.6 | 96.2 | 48.6 | 34.6 | 7.3 | 65.4 | 92.7 | 17.2 | 99.1 | 81.4 | 84.6 | 0.5137 |
| 0.22 | 178 | 90 | 93 | 7 | 72.8 | 96.2 | 49.2 | 34.3 | 7.2 | 65.7 | 92.8 | 17.4 | 99.2 | 81.5 | 84.8 | 0.5082 |
| 0.23 | 178 | 91 | 92 | 7 | 73.1 | 96.2 | 49.7 | 34.1 | 7.1 | 65.9 | 92.9 | 17.5 | 99.2 | 81.7 | 84.9 | 0.5027 |
| 0.24 | 176 | 93 | 90 | 9 | 73.1 | 95.1 | 50.8 | 33.8 | 8.8 | 66.2 | 91.2 | 17.7 | 98.9 | 81.9 | 81.7 | 0.4918 |
| 0.25 | 174 | 93 | 90 | 11 | 72.6 | 94.1 | 50.8 | 34.1 | 10.6 | 65.9 | 89.4 | 17.5 | 98.7 | 81.7 | 78.6 | 0.4918 |
| 0.26 | 174 | 99 | 84 | 11 | 74.2 | 94.1 | 54.1 | 32.6 | 10 | 67.4 | 90 | 18.5 | 98.8 | 82.7 | 79.6 | 0.459 |
| 0.27 | 173 | 105 | 78 | 12 | 75.5 | 93.5 | 57.4 | 31.1 | 10.3 | 68.9 | 89.7 | 19.6 | 98.8 | 83.7 | 79.1 | 0.4262 |
| 0.28 | 173 | 109 | 74 | 12 | 76.6 | 93.5 | 59.6 | 30 | 9.9 | 70 | 90.1 | 20.4 | 98.8 | 84.4 | 79.7 | 0.4044 |
| 0.29 | 172 | 114 | 69 | 13 | 77.7 | 93 | 62.3 | 28.6 | 10.2 | 71.4 | 89.8 | 21.5 | 98.8 | 85.2 | 79.2 | 0.377 |
| 0.3 | 171 | 116 | 67 | 14 | 78 | 92.4 | 63.4 | 28.2 | 10.8 | 71.8 | 89.2 | 21.9 | 98.7 | 85.5 | 78.2 | 0.3661 |
| 0.31 | 171 | 117 | 66 | 14 | 78.3 | 92.4 | 63.9 | 27.8 | 10.7 | 72.2 | 89.3 | 22.2 | 98.7 | 85.7 | 78.4 | 0.3607 |
| 0.32 | 167 | 117 | 66 | 18 | 77.2 | 90.3 | 63.9 | 28.3 | 13.3 | 71.7 | 86.7 | 21.8 | 98.3 | 85.4 | 73.8 | 0.3607 |
| 0.33 | 165 | 119 | 64 | 20 | 77.2 | 89.2 | 65 | 27.9 | 14.4 | 72.1 | 85.6 | 22.1 | 98.2 | 85.6 | 72.1 | 0.3497 |
| 0.34 | 165 | 119 | 64 | 20 | 77.2 | 89.2 | 65 | 27.9 | 14.4 | 72.1 | 85.6 | 22.1 | 98.2 | 85.6 | 72.1 | 0.3497 |
| 0.35 | 164 | 121 | 62 | 21 | 77.4 | 88.6 | 66.1 | 27.4 | 14.8 | 72.6 | 85.2 | 22.5 | 98.1 | 85.9 | 71.4 | 0.3388 |
| 0.36 | 162 | 124 | 59 | 23 | 77.7 | 87.6 | 67.8 | 26.7 | 15.6 | 73.3 | 84.4 | 23.2 | 98 | 86.4 | 70 | 0.3224 |
| 0.37 | 161 | 126 | 57 | 24 | 78 | 87 | 68.9 | 26.1 | 16 | 73.9 | 84 | 23.7 | 97.9 | 86.7 | 69.5 | 0.3115 |
| 0.38 | 159 | 127 | 56 | 26 | 77.7 | 85.9 | 69.4 | 26 | 17 | 74 | 83 | 23.8 | 97.8 | 86.8 | 67.9 | 0.306 |
| 0.39 | 159 | 129 | 54 | 26 | 78.3 | 85.9 | 70.5 | 25.4 | 16.8 | 74.6 | 83.2 | 24.4 | 97.8 | 87.2 | 68.3 | 0.2951 |
| 0.4 | 159 | 131 | 52 | 26 | 78.8 | 85.9 | 71.6 | 24.6 | 16.6 | 75.4 | 83.4 | 25.2 | 97.9 | 87.6 | 68.6 | 0.2842 |
| 0.41 | 156 | 133 | 50 | 29 | 78.5 | 84.3 | 72.7 | 24.3 | 17.9 | 75.7 | 82.1 | 25.5 | 97.7 | 87.8 | 66.5 | 0.2732 |
| 0.42 | 155 | 133 | 50 | 30 | 78.3 | 83.8 | 72.7 | 24.4 | 18.4 | 75.6 | 81.6 | 25.4 | 97.6 | 87.7 | 65.8 | 0.2732 |
| 0.43 | 155 | 136 | 47 | 30 | 79.1 | 83.8 | 74.3 | 23.3 | 18.1 | 76.7 | 81.9 | 26.6 | 97.6 | 88.4 | 66.3 | 0.2568 |
| 0.44 | 153 | 137 | 46 | 32 | 78.8 | 82.7 | 74.9 | 23.1 | 18.9 | 76.9 | 81.1 | 26.8 | 97.5 | 88.5 | 65 | 0.2514 |
| 0.45 | 151 | 138 | 45 | 34 | 78.5 | 81.6 | 75.4 | 23 | 19.8 | 77 | 80.2 | 26.9 | 97.4 | 88.6 | 63.7 | 0.2459 |
| 0.46 | 151 | 142 | 41 | 34 | 79.6 | 81.6 | 77.6 | 21.4 | 19.3 | 78.6 | 80.7 | 28.8 | 97.4 | 89.5 | 64.4 | 0.224 |
| 0.47 | 149 | 144 | 39 | 36 | 79.6 | 80.5 | 78.7 | 20.7 | 20 | 79.3 | 80 | 29.6 | 97.3 | 89.8 | 63.4 | 0.2131 |
| 0.48 | 147 | 146 | 37 | 38 | 79.6 | 79.5 | 79.8 | 20.1 | 20.7 | 79.9 | 79.3 | 30.4 | 97.2 | 90.2 | 62.5 | 0.2022 |
| 0.49 | 146 | 149 | 34 | 39 | 80.2 | 78.9 | 81.4 | 18.9 | 20.7 | 81.1 | 79.3 | 32.1 | 97.2 | 90.8 | 62.3 | 0.1858 |
| 0.5 | 145 | 149 | 34 | 40 | 79.9 | 78.4 | 81.4 | 19 | 21.2 | 81 | 78.8 | 31.9 | 97.1 | 90.8 | 61.7 | 0.1858 |
| 0.51 | 144 | 152 | 31 | 41 | 80.4 | 77.8 | 83.1 | 17.7 | 21.2 | 82.3 | 78.8 | 33.8 | 97.1 | 91.5 | 61.6 | 0.1694 |
| 0.52 | 144 | 152 | 31 | 41 | 80.4 | 77.8 | 83.1 | 17.7 | 21.2 | 82.3 | 78.8 | 33.8 | 97.1 | 91.5 | 61.6 | 0.1694 |
| 0.53 | 142 | 153 | 30 | 43 | 80.2 | 76.8 | 83.6 | 17.4 | 21.9 | 82.6 | 78.1 | 34.2 | 97 | 91.6 | 60.7 | 0.1639 |
| 0.54 | 140 | 156 | 27 | 45 | 80.4 | 75.7 | 85.2 | 16.2 | 22.4 | 83.8 | 77.6 | 36.3 | 96.9 | 92.3 | 60 | 0.1475 |
| 0.55 | 139 | 157 | 26 | 46 | 80.4 | 75.1 | 85.8 | 15.8 | 22.7 | 84.2 | 77.3 | 37 | 96.9 | 92.5 | 59.7 | 0.1421 |
| 0.56 | 137 | 158 | 25 | 48 | 80.2 | 74.1 | 86.3 | 15.4 | 23.3 | 84.6 | 76.7 | 37.6 | 96.8 | 92.7 | 58.8 | 0.1366 |
| 0.57 | 136 | 158 | 25 | 49 | 79.9 | 73.5 | 86.3 | 15.5 | 23.7 | 84.5 | 76.3 | 37.4 | 96.7 | 92.6 | 58.3 | 0.1366 |
| 0.58 | 135 | 158 | 25 | 50 | 79.6 | 73 | 86.3 | 15.6 | 24 | 84.4 | 76 | 37.2 | 96.6 | 92.6 | 57.8 | 0.1366 |
| 0.59 | 134 | 159 | 24 | 51 | 79.6 | 72.4 | 86.9 | 15.2 | 24.3 | 84.8 | 75.7 | 38 | 96.6 | 92.8 | 57.5 | 0.1311 |
| 0.6 | 134 | 161 | 22 | 51 | 80.2 | 72.4 | 88 | 14.1 | 24.1 | 85.9 | 75.9 | 40.1 | 96.6 | 93.4 | 57.8 | 0.1202 |
| 0.61 | 131 | 161 | 22 | 54 | 79.3 | 70.8 | 88 | 14.4 | 25.1 | 85.6 | 74.9 | 39.6 | 96.4 | 93.2 | 56.4 | 0.1202 |
| 0.62 | 130 | 162 | 21 | 55 | 79.3 | 70.3 | 88.5 | 13.9 | 25.3 | 86.1 | 74.7 | 40.5 | 96.4 | 93.5 | 56.1 | 0.1148 |
| 0.63 | 127 | 165 | 18 | 58 | 79.3 | 68.6 | 90.2 | 12.4 | 26 | 87.6 | 74 | 43.7 | 96.3 | 94.2 | 55.2 | 0.0984 |
| 0.64 | 126 | 167 | 16 | 59 | 79.6 | 68.1 | 91.3 | 11.3 | 26.1 | 88.7 | 73.9 | 46.4 | 96.3 | 94.8 | 55.1 | 0.0874 |
| 0.65 | 126 | 169 | 14 | 59 | 80.2 | 68.1 | 92.3 | 10 | 25.9 | 90 | 74.1 | 49.7 | 96.3 | 95.4 | 55.4 | 0.0765 |
| 0.66 | 123 | 170 | 13 | 62 | 79.6 | 66.5 | 92.9 | 9.6 | 26.7 | 90.4 | 73.3 | 51 | 96.1 | 95.6 | 54.3 | 0.071 |
| 0.67 | 123 | 170 | 13 | 62 | 79.6 | 66.5 | 92.9 | 9.6 | 26.7 | 90.4 | 73.3 | 51 | 96.1 | 95.6 | 54.3 | 0.071 |
| 0.68 | 119 | 170 | 13 | 66 | 78.5 | 64.3 | 92.9 | 9.8 | 28 | 90.2 | 72 | 50.2 | 95.9 | 95.5 | 52.7 | 0.071 |
| 0.69 | 117 | 170 | 13 | 68 | 78 | 63.2 | 92.9 | 10 | 28.6 | 90 | 71.4 | 49.7 | 95.8 | 95.4 | 52 | 0.071 |
| 0.7 | 116 | 170 | 13 | 69 | 77.7 | 62.7 | 92.9 | 10.1 | 28.9 | 89.9 | 71.1 | 49.5 | 95.7 | 95.4 | 51.6 | 0.071 |
| 0.71 | 115 | 170 | 13 | 70 | 77.4 | 62.2 | 92.9 | 10.2 | 29.2 | 89.8 | 70.8 | 49.3 | 95.7 | 95.3 | 51.3 | 0.071 |
| 0.72 | 113 | 171 | 12 | 72 | 77.2 | 61.1 | 93.4 | 9.6 | 29.6 | 90.4 | 70.4 | 50.9 | 95.6 | 95.6 | 50.7 | 0.0656 |
| 0.73 | 111 | 171 | 12 | 74 | 76.6 | 60 | 93.4 | 9.8 | 30.2 | 90.2 | 69.8 | 50.4 | 95.5 | 95.5 | 50 | 0.0656 |
| 0.74 | 107 | 171 | 12 | 78 | 75.5 | 57.8 | 93.4 | 10.1 | 31.3 | 89.9 | 68.7 | 49.5 | 95.2 | 95.4 | 48.7 | 0.0656 |
| 0.75 | 105 | 172 | 11 | 80 | 75.3 | 56.8 | 94 | 9.5 | 31.7 | 90.5 | 68.3 | 51.2 | 95.1 | 95.7 | 48.2 | 0.0601 |
| 0.76 | 99 | 173 | 10 | 86 | 73.9 | 53.5 | 94.5 | 9.2 | 33.2 | 90.8 | 66.8 | 52.1 | 94.8 | 95.8 | 46.6 | 0.0546 |
| 0.77 | 96 | 173 | 10 | 89 | 73.1 | 51.9 | 94.5 | 9.4 | 34 | 90.6 | 66 | 51.3 | 94.6 | 95.7 | 45.7 | 0.0546 |
| 0.78 | 95 | 173 | 10 | 90 | 72.8 | 51.4 | 94.5 | 9.5 | 34.2 | 90.5 | 65.8 | 51.1 | 94.6 | 95.6 | 45.4 | 0.0546 |
| 0.79 | 93 | 173 | 10 | 92 | 72.3 | 50.3 | 94.5 | 9.7 | 34.7 | 90.3 | 65.3 | 50.5 | 94.5 | 95.5 | 44.9 | 0.0546 |
| 0.8 | 92 | 175 | 8 | 93 | 72.6 | 49.7 | 95.6 | 8 | 34.7 | 92 | 65.3 | 55.8 | 94.5 | 96.4 | 44.9 | 0.0437 |
| 0.81 | 91 | 175 | 8 | 94 | 72.3 | 49.2 | 95.6 | 8.1 | 34.9 | 91.9 | 65.1 | 55.6 | 94.4 | 96.3 | 44.6 | 0.0437 |
| 0.82 | 89 | 175 | 8 | 96 | 71.7 | 48.1 | 95.6 | 8.2 | 35.4 | 91.8 | 64.6 | 55 | 94.3 | 96.3 | 44.1 | 0.0437 |
| 0.83 | 87 | 176 | 7 | 98 | 71.5 | 47 | 96.2 | 7.4 | 35.8 | 92.6 | 64.2 | 57.7 | 94.2 | 96.6 | 43.8 | 0.0383 |
| 0.84 | 86 | 177 | 6 | 99 | 71.5 | 46.5 | 96.7 | 6.5 | 35.9 | 93.5 | 64.1 | 61.2 | 94.2 | 97.1 | 43.6 | 0.0328 |
| 0.85 | 83 | 178 | 5 | 102 | 70.9 | 44.9 | 97.3 | 5.7 | 36.4 | 94.3 | 63.6 | 64.6 | 94.1 | 97.5 | 43.1 | 0.0273 |
| 0.86 | 79 | 178 | 5 | 106 | 69.8 | 42.7 | 97.3 | 6 | 37.3 | 94 | 62.7 | 63.5 | 93.9 | 97.3 | 42.1 | 0.0273 |
| 0.87 | 74 | 178 | 5 | 111 | 68.5 | 40 | 97.3 | 6.3 | 38.4 | 93.7 | 61.6 | 61.9 | 93.6 | 97.2 | 41 | 0.0273 |
| 0.88 | 71 | 178 | 5 | 114 | 67.7 | 38.4 | 97.3 | 6.6 | 39 | 93.4 | 61 | 60.9 | 93.4 | 97 | 40.4 | 0.0273 |
| 0.89 | 67 | 179 | 4 | 118 | 66.8 | 36.2 | 97.8 | 5.6 | 39.7 | 94.4 | 60.3 | 64.8 | 93.2 | 97.5 | 39.7 | 0.0219 |
| 0.9 | 67 | 180 | 3 | 118 | 67.1 | 36.2 | 98.4 | 4.3 | 39.6 | 95.7 | 60.4 | 71.1 | 93.3 | 98.1 | 39.8 | 0.0164 |
| 0.91 | 67 | 180 | 3 | 118 | 67.1 | 36.2 | 98.4 | 4.3 | 39.6 | 95.7 | 60.4 | 71.1 | 93.3 | 98.1 | 39.8 | 0.0164 |
| 0.92 | 64 | 180 | 3 | 121 | 66.3 | 34.6 | 98.4 | 4.5 | 40.2 | 95.5 | 59.8 | 70.1 | 93.1 | 98 | 39.2 | 0.0164 |
| 0.93 | 60 | 181 | 2 | 125 | 65.5 | 32.4 | 98.9 | 3.2 | 40.8 | 96.8 | 59.2 | 76.7 | 92.9 | 98.6 | 38.6 | 0.0109 |
| 0.94 | 57 | 181 | 2 | 128 | 64.7 | 30.8 | 98.9 | 3.4 | 41.4 | 96.6 | 58.6 | 75.8 | 92.8 | 98.5 | 38 | 0.0109 |
| 0.95 | 53 | 181 | 2 | 132 | 63.6 | 28.6 | 98.9 | 3.6 | 42.2 | 96.4 | 57.8 | 74.4 | 92.6 | 98.4 | 37.3 | 0.0109 |
| 0.96 | 47 | 181 | 2 | 138 | 62 | 25.4 | 98.9 | 4.1 | 43.3 | 95.9 | 56.7 | 72.1 | 92.3 | 98.2 | 36.2 | 0.0109 |
| 0.97 | 41 | 183 | 0 | 144 | 60.9 | 22.2 | 100 | 0 | 44 | 100 | 56 | 100 | 92 | 100 | 35.5 | 0 |
| 0.98 | 37 | 183 | 0 | 148 | 59.8 | 20 | 100 | 0 | 44.7 | 100 | 55.3 | 100 | 91.8 | 100 | 34.9 | 0 |
| 0.99 | 29 | 183 | 0 | 156 | 57.6 | 15.7 | 100 | 0 | 46 | 100 | 54 | 100 | 91.4 | 100 | 33.7 | 0 |
| 1 | 0 | 183 | 0 | 185 | 49.7 | 0 | 100 | — | 50.3 | — | 49.7 | — | 90 | — | 30 | 0 |

TABLE 9D
Analysis of Training plus Validation sets excluding Singlets using Five Variable Model; n = 268;
Stage 1 (evt) vs. Stage 0 (non-evt).

| | | | | | | | | | | Prevalence | | | | | | |
|---|---|---|---|---|---|---|---|---|---|---|---|---|---|---|---|---|
| | correct | | incorrect | | % | | | | | 54.90% | | 10.0% | | 70% | | |
| prob | evt | n-e | evt | n-e | accuracy | sensi | spec | f-pos | f-neg | PPV | NPV | PPV | NPV | PPV | NPV | 1-Spec |
| 0 | 147 | 0 | 121 | 0 | 54.9 | 100 | 0 | 45.1 | — | 54.9 | — | 10 | — | 70 | — | 1 |
| 0.01 | 147 | 0 | 121 | 0 | 54.9 | 100 | 0 | 45.1 | — | 54.9 | — | 10 | — | 70 | — | 1 |
| 0.02 | 147 | 1 | 120 | 0 | 55.2 | 100 | 0.8 | 44.9 | 0 | 55.1 | 100 | 10.1 | 100 | 70.2 | 100 | 0.9917 |
| 0.03 | 147 | 3 | 118 | 0 | 56 | 100 | 2.5 | 44.5 | 0 | 55.5 | 100 | 10.2 | 100 | 70.5 | 100 | 0.9752 |
| 0.04 | 146 | 7 | 114 | 1 | 57.1 | 99.3 | 5.8 | 43.8 | 12.5 | 56.2 | 87.5 | 10.5 | 98.7 | 71.1 | 78.5 | 0.9421 |
| 0.05 | 146 | 8 | 113 | 1 | 57.5 | 99.3 | 6.6 | 43.6 | 11.1 | 56.4 | 88.9 | 10.6 | 98.9 | 71.3 | 80.6 | 0.9339 |
| 0.06 | 145 | 14 | 107 | 2 | 59.3 | 98.6 | 11.6 | 42.5 | 12.5 | 57.5 | 87.5 | 11 | 98.7 | 72.2 | 78.5 | 0.8843 |
| 0.07 | 145 | 15 | 106 | 2 | 59.7 | 98.6 | 12.4 | 42.2 | 11.8 | 57.8 | 88.2 | 11.1 | 98.8 | 72.4 | 79.6 | 0.876 |
| 0.08 | 145 | 17 | 104 | 2 | 60.4 | 98.6 | 14 | 41.8 | 10.5 | 58.2 | 89.5 | 11.3 | 98.9 | 72.8 | 81.6 | 0.8595 |
| 0.09 | 145 | 23 | 98 | 2 | 62.7 | 98.6 | 19 | 40.3 | 8 | 59.7 | 92 | 11.9 | 99.2 | 74 | 85.7 | 0.8099 |
| 0.1 | 144 | 29 | 92 | 3 | 64.6 | 98 | 24 | 39 | 9.4 | 61 | 90.6 | 12.5 | 99.1 | 75 | 83.4 | 0.7603 |
| 0.11 | 144 | 30 | 91 | 3 | 64.9 | 98 | 24.8 | 38.7 | 9.1 | 61.3 | 90.9 | 12.6 | 99.1 | 75.2 | 83.9 | 0.7521 |
| 0.12 | 144 | 38 | 83 | 3 | 67.9 | 98 | 31.4 | 36.6 | 7.3 | 63.4 | 92.7 | 13.7 | 99.3 | 76.9 | 86.8 | 0.686 |
| 0.13 | 144 | 41 | 80 | 3 | 69 | 98 | 33.9 | 35.7 | 6.8 | 64.3 | 93.2 | 14.1 | 99.3 | 77.6 | 87.7 | 0.6612 |
| 0.14 | 144 | 43 | 78 | 3 | 69.8 | 98 | 35.5 | 35.1 | 6.5 | 64.9 | 93.5 | 14.4 | 99.4 | 78 | 88.2 | 0.6446 |
| 0.15 | 144 | 49 | 72 | 3 | 72 | 98 | 40.5 | 33.3 | 5.8 | 66.7 | 94.2 | 15.5 | 99.4 | 79.3 | 89.5 | 0.595 |
| 0.16 | 143 | 49 | 72 | 4 | 71.6 | 97.3 | 40.5 | 33.5 | 7.5 | 66.5 | 92.5 | 15.4 | 99.3 | 79.2 | 86.4 | 0.595 |
| 0.17 | 143 | 51 | 70 | 4 | 72.4 | 97.3 | 42.1 | 32.9 | 7.3 | 67.1 | 92.7 | 15.7 | 99.3 | 79.7 | 86.9 | 0.5785 |
| 0.18 | 143 | 54 | 67 | 4 | 73.5 | 97.3 | 44.6 | 31.9 | 6.9 | 68.1 | 93.1 | 16.3 | 99.3 | 80.4 | 87.5 | 0.5537 |
| 0.19 | 143 | 56 | 65 | 4 | 74.3 | 97.3 | 46.3 | 31.3 | 6.7 | 68.8 | 93.3 | 16.8 | 99.4 | 80.9 | 87.9 | 0.5372 |
| 0.2 | 143 | 58 | 63 | 4 | 75 | 97.3 | 47.9 | 30.6 | 6.5 | 69.4 | 93.5 | 17.2 | 99.4 | 81.3 | 88.3 | 0.5207 |
| 0.21 | 143 | 61 | 60 | 4 | 76.1 | 97.3 | 50.4 | 29.6 | 6.2 | 70.4 | 93.8 | 17.9 | 99.4 | 82.1 | 88.8 | 0.4959 |
| 0.22 | 143 | 61 | 60 | 4 | 76.1 | 97.3 | 50.4 | 29.6 | 6.2 | 70.4 | 93.8 | 17.9 | 99.4 | 82.1 | 88.8 | 0.4959 |
| 0.23 | 143 | 61 | 60 | 4 | 76.1 | 97.3 | 50.4 | 29.6 | 6.2 | 70.4 | 93.8 | 17.9 | 99.4 | 82.1 | 88.8 | 0.4959 |
| 0.24 | 143 | 62 | 59 | 4 | 76.5 | 97.3 | 51.2 | 29.2 | 6.1 | 70.8 | 93.9 | 18.1 | 99.4 | 82.3 | 89 | 0.4876 |
| 0.25 | 141 | 62 | 59 | 6 | 75.7 | 95.9 | 51.2 | 29.5 | 8.8 | 70.5 | 91.2 | 17.9 | 99.1 | 82.1 | 84.3 | 0.4876 |
| 0.26 | 141 | 67 | 54 | 6 | 77.6 | 95.9 | 55.4 | 27.7 | 8.2 | 72.3 | 91.8 | 19.3 | 99.2 | 83.4 | 85.3 | 0.4463 |
| 0.27 | 140 | 70 | 51 | 7 | 78.4 | 95.2 | 57.9 | 26.7 | 9.1 | 73.3 | 90.9 | 20.1 | 99.1 | 84.1 | 83.9 | 0.4215 |
| 0.28 | 140 | 72 | 49 | 7 | 79.1 | 95.2 | 59.5 | 25.9 | 8.9 | 74.1 | 91.1 | 20.7 | 99.1 | 84.6 | 84.3 | 0.405 |
| 0.29 | 140 | 75 | 46 | 7 | 80.2 | 95.2 | 62 | 24.7 | 8.5 | 75.3 | 91.5 | 21.8 | 99.2 | 85.4 | 84.8 | 0.3802 |
| 0.3 | 139 | 77 | 44 | 8 | 80.6 | 94.6 | 63.6 | 24 | 9.4 | 76 | 90.6 | 22.4 | 99.1 | 85.9 | 83.4 | 0.3636 |
| 0.31 | 139 | 78 | 43 | 8 | 81 | 94.6 | 64.5 | 23.6 | 9.3 | 76.4 | 90.7 | 22.8 | 99.1 | 86.1 | 83.5 | 0.3554 |
| 0.32 | 138 | 78 | 43 | 9 | 80.6 | 93.9 | 64.5 | 23.8 | 10.3 | 76.2 | 89.7 | 22.7 | 99 | 86 | 81.9 | 0.3554 |
| 0.33 | 137 | 79 | 42 | 10 | 80.6 | 93.2 | 65.3 | 23.5 | 11.2 | 76.5 | 88.8 | 23 | 98.9 | 86.2 | 80.4 | 0.3471 |
| 0.34 | 137 | 79 | 42 | 10 | 80.6 | 93.2 | 65.3 | 23.5 | 11.2 | 76.5 | 88.8 | 23 | 98.9 | 86.2 | 80.4 | 0.3471 |
| 0.35 | 137 | 80 | 41 | 10 | 81 | 93.2 | 66.1 | 23 | 11.1 | 77 | 88.9 | 23.4 | 98.9 | 86.5 | 80.6 | 0.3388 |
| 0.36 | 136 | 82 | 39 | 11 | 81.3 | 92.5 | 67.8 | 22.3 | 11.8 | 77.7 | 88.2 | 24.2 | 98.8 | 87 | 79.5 | 0.3223 |
| 0.37 | 135 | 83 | 38 | 12 | 81.3 | 91.8 | 68.6 | 22 | 12.6 | 78 | 87.4 | 24.5 | 98.7 | 87.2 | 78.3 | 0.314 |
| 0.38 | 134 | 83 | 38 | 13 | 81 | 91.2 | 68.6 | 22.1 | 13.5 | 77.9 | 86.5 | 24.4 | 98.6 | 87.1 | 76.9 | 0.314 |
| 0.39 | 134 | 85 | 36 | 13 | 81.7 | 91.2 | 70.2 | 21.2 | 13.3 | 78.8 | 86.7 | 25.4 | 98.6 | 87.7 | 77.3 | 0.2975 |
| 0.4 | 134 | 85 | 36 | 13 | 81.7 | 91.2 | 70.2 | 21.2 | 13.3 | 78.8 | 86.7 | 25.4 | 98.6 | 87.7 | 77.3 | 0.2975 |
| 0.41 | 132 | 86 | 35 | 15 | 81.3 | 89.8 | 71.1 | 21 | 14.9 | 79 | 85.1 | 25.6 | 98.4 | 87.9 | 74.9 | 0.2893 |
| 0.42 | 131 | 86 | 35 | 16 | 81 | 89.1 | 71.1 | 21.1 | 15.7 | 78.9 | 84.3 | 25.5 | 98.3 | 87.8 | 73.7 | 0.2893 |
| 0.43 | 131 | 88 | 33 | 16 | 81.7 | 89.1 | 72.7 | 20.1 | 15.4 | 79.9 | 84.6 | 26.6 | 98.4 | 88.4 | 74.1 | 0.2727 |
| 0.44 | 129 | 89 | 32 | 18 | 81.3 | 87.8 | 73.6 | 19.9 | 16.8 | 80.1 | 83.2 | 26.9 | 98.2 | 88.6 | 72 | 0.2645 |
| 0.45 | 127 | 90 | 31 | 20 | 81 | 86.4 | 74.4 | 19.6 | 18.2 | 80.4 | 81.8 | 27.3 | 98 | 88.7 | 70.1 | 0.2562 |
| 0.46 | 127 | 93 | 28 | 20 | 82.1 | 86.4 | 76.9 | 18.1 | 17.7 | 81.9 | 82.3 | 29.3 | 98.1 | 89.7 | 70.8 | 0.2314 |
| 0.47 | 126 | 95 | 26 | 21 | 82.5 | 85.7 | 78.5 | 17.1 | 18.1 | 82.9 | 81.9 | 30.7 | 98 | 90.3 | 70.2 | 0.2149 |
| 0.48 | 126 | 96 | 25 | 21 | 82.8 | 85.7 | 79.3 | 16.6 | 17.9 | 83.4 | 82.1 | 31.6 | 98 | 90.6 | 70.4 | 0.2066 |
| 0.49 | 126 | 99 | 22 | 21 | 84 | 85.7 | 81.8 | 14.9 | 17.5 | 85.1 | 82.5 | 34.4 | 98.1 | 91.7 | 71.1 | 0.1818 |
| 0.5 | 125 | 99 | 22 | 22 | 83.6 | 85 | 81.8 | 15 | 18.2 | 85 | 81.8 | 34.2 | 98 | 91.6 | 70.1 | 0.1818 |
| 0.51 | 124 | 102 | 19 | 23 | 84.3 | 84.4 | 84.3 | 13.3 | 18.4 | 86.7 | 81.6 | 37.4 | 98 | 92.6 | 69.8 | 0.157 |
| 0.52 | 124 | 102 | 19 | 23 | 84.3 | 84.4 | 84.3 | 13.3 | 18.4 | 86.7 | 81.6 | 37.4 | 98 | 92.6 | 69.8 | 0.157 |
| 0.53 | 122 | 102 | 19 | 25 | 83.6 | 83 | 84.3 | 13.5 | 19.7 | 86.5 | 80.3 | 37 | 97.8 | 92.5 | 68 | 0.157 |
| 0.54 | 121 | 103 | 18 | 26 | 83.6 | 82.3 | 85.1 | 12.9 | 20.2 | 87.1 | 79.8 | 38.1 | 97.7 | 92.8 | 67.3 | 0.1488 |
| 0.55 | 120 | 103 | 18 | 27 | 83.2 | 81.6 | 85.1 | 13 | 20.8 | 87 | 79.2 | 37.9 | 97.7 | 92.8 | 66.5 | 0.1488 |
| 0.56 | 119 | 104 | 17 | 28 | 83.2 | 81 | 86 | 12.5 | 21.2 | 87.5 | 78.8 | 39 | 97.6 | 93.1 | 65.9 | 0.1405 |
| 0.57 | 118 | 104 | 17 | 29 | 82.8 | 80.3 | 86 | 12.6 | 21.8 | 87.4 | 78.2 | 38.8 | 97.5 | 93 | 65.1 | 0.1405 |
| 0.58 | 118 | 104 | 17 | 29 | 82.8 | 80.3 | 86 | 12.6 | 21.8 | 87.4 | 78.2 | 38.8 | 97.5 | 93 | 65.1 | 0.1405 |
| 0.59 | 118 | 104 | 17 | 29 | 82.8 | 80.3 | 86 | 12.6 | 21.8 | 87.4 | 78.2 | 38.8 | 97.5 | 93 | 65.1 | 0.1405 |
| 0.6 | 118 | 105 | 16 | 29 | 83.2 | 80.3 | 86.8 | 11.9 | 21.6 | 88.1 | 78.4 | 40.3 | 97.5 | 93.4 | 65.3 | 0.1322 |
| 0.61 | 115 | 105 | 16 | 32 | 82.1 | 78.2 | 86.8 | 12.2 | 23.4 | 87.8 | 76.6 | 39.7 | 97.3 | 93.2 | 63.1 | 0.1322 |
| 0.62 | 115 | 105 | 16 | 32 | 82.1 | 78.2 | 86.8 | 12.2 | 23.4 | 87.8 | 76.6 | 39.7 | 97.3 | 93.2 | 63.1 | 0.1322 |
| 0.63 | 113 | 107 | 14 | 34 | 82.1 | 76.9 | 88.4 | 11 | 24.1 | 89 | 75.9 | 42.5 | 97.2 | 93.9 | 62.1 | 0.1157 |
| 0.64 | 113 | 108 | 13 | 34 | 82.5 | 76.9 | 89.3 | 10.3 | 23.9 | 89.7 | 76.1 | 44.3 | 97.2 | 94.3 | 62.3 | 0.1074 |
| 0.65 | 113 | 110 | 11 | 34 | 83.2 | 76.9 | 90.9 | 8.9 | 23.6 | 91.1 | 76.4 | 48.4 | 97.3 | 95.2 | 62.7 | 0.0909 |
| 0.66 | 111 | 110 | 11 | 36 | 82.5 | 75.5 | 90.9 | 9 | 24.7 | 91 | 75.3 | 48 | 97.1 | 95.1 | 61.4 | 0.0909 |
| 0.67 | 111 | 110 | 11 | 36 | 82.5 | 75.5 | 90.9 | 9 | 24.7 | 91 | 75.3 | 48 | 97.1 | 95.1 | 61.4 | 0.0909 |
| 0.68 | 109 | 110 | 11 | 38 | 81.7 | 74.1 | 90.9 | 9.2 | 25.7 | 90.8 | 74.3 | 47.5 | 96.9 | 95 | 60.1 | 0.0909 |
| 0.69 | 107 | 110 | 11 | 40 | 81 | 72.8 | 90.9 | 9.3 | 26.7 | 90.7 | 73.3 | 47.1 | 96.8 | 94.9 | 58.9 | 0.0909 |
| 0.7 | 106 | 110 | 11 | 41 | 80.6 | 72.1 | 90.9 | 9.4 | 27.2 | 90.6 | 72.8 | 46.8 | 96.7 | 94.9 | 58.3 | 0.0909 |
| 0.71 | 106 | 110 | 11 | 41 | 80.6 | 72.1 | 90.9 | 9.4 | 27.2 | 90.6 | 72.8 | 46.8 | 96.7 | 94.9 | 58.3 | 0.0909 |
| 0.72 | 104 | 110 | 11 | 43 | 79.9 | 70.7 | 90.9 | 9.6 | 28.1 | 90.4 | 71.9 | 46.4 | 96.5 | 94.8 | 57.1 | 0.0909 |
| 0.73 | 102 | 110 | 11 | 45 | 79.1 | 69.4 | 90.9 | 9.7 | 29 | 90.3 | 71 | 45.9 | 96.4 | 94.7 | 56 | 0.0909 |
| 0.74 | 98 | 110 | 11 | 49 | 77.6 | 66.7 | 90.9 | 10.1 | 30.8 | 89.9 | 69.2 | 44.9 | 96.1 | 94.5 | 53.9 | 0.0909 |
| 0.75 | 96 | 111 | 10 | 51 | 77.2 | 65.3 | 91.7 | 9.4 | 31.5 | 90.6 | 68.5 | 46.8 | 96 | 94.9 | 53.1 | 0.0826 |
| 0.76 | 91 | 111 | 10 | 56 | 75.4 | 61.9 | 91.7 | 9.9 | 33.5 | 90.1 | 66.5 | 45.4 | 95.6 | 94.6 | 50.8 | 0.0826 |
| 0.77 | 89 | 111 | 10 | 58 | 74.6 | 60.5 | 91.7 | 10.1 | 34.3 | 89.9 | 65.7 | 44.9 | 95.4 | 94.5 | 49.9 | 0.0826 |
| 0.78 | 88 | 111 | 10 | 59 | 74.3 | 59.9 | 91.7 | 10.2 | 34.7 | 89.8 | 65.3 | 44.6 | 95.4 | 94.4 | 49.5 | 0.0826 |
| 0.79 | 86 | 111 | 10 | 61 | 73.5 | 58.5 | 91.7 | 10.4 | 35.5 | 89.6 | 64.5 | 44 | 95.2 | 94.3 | 48.7 | 0.0826 |
| 0.8 | 85 | 113 | 8 | 62 | 73.9 | 57.8 | 93.4 | 8.6 | 35.4 | 91.4 | 64.6 | 49.3 | 95.2 | 95.3 | 48.7 | 0.0661 |
| 0.81 | 84 | 113 | 8 | 63 | 73.5 | 57.1 | 93.4 | 8.7 | 35.8 | 91.3 | 64.2 | 49 | 95.1 | 95.3 | 48.3 | 0.0661 |
| 0.82 | 82 | 113 | 8 | 65 | 72.8 | 55.8 | 93.4 | 8.9 | 36.5 | 91.1 | 63.5 | 48.4 | 95 | 95.2 | 47.5 | 0.0661 |
| 0.83 | 80 | 114 | 7 | 67 | 72.4 | 54.4 | 94.2 | 8 | 37 | 92 | 63 | 51.1 | 94.9 | 95.6 | 47 | 0.0579 |
| 0.84 | 80 | 115 | 6 | 67 | 72.8 | 54.4 | 95 | 7 | 36.8 | 93 | 63.2 | 54.9 | 94.9 | 96.2 | 47.2 | 0.0496 |
| 0.85 | 79 | 116 | 5 | 68 | 72.8 | 53.7 | 95.9 | 6 | 37 | 94 | 63 | 59.1 | 94.9 | 96.8 | 47 | 0.0413 |
| 0.86 | 75 | 116 | 5 | 72 | 71.3 | 51 | 95.9 | 6.3 | 38.3 | 93.8 | 61.7 | 57.8 | 94.6 | 96.6 | 45.6 | 0.0413 |
| 0.87 | 70 | 116 | 5 | 77 | 69.4 | 47.6 | 95.9 | 6.7 | 39.9 | 93.3 | 60.1 | 56.1 | 94.3 | 96.4 | 44 | 0.0413 |
| 0.88 | 67 | 116 | 5 | 80 | 68.3 | 45.6 | 95.9 | 6.9 | 40.8 | 93.1 | 59.2 | 55.1 | 94.1 | 96.3 | 43 | 0.0413 |
| 0.89 | 64 | 117 | 4 | 83 | 67.5 | 43.5 | 96.7 | 5.9 | 41.5 | 94.1 | 58.5 | 59.4 | 93.9 | 96.8 | 42.3 | 0.0331 |
| 0.9 | 64 | 118 | 3 | 83 | 67.9 | 43.5 | 97.5 | 4.5 | 41.3 | 95.5 | 58.7 | 66.1 | 94 | 97.6 | 42.5 | 0.0248 |
| 0.91 | 64 | 118 | 3 | 83 | 67.9 | 43.5 | 97.5 | 4.5 | 41.3 | 95.5 | 58.7 | 66.1 | 94 | 97.6 | 42.5 | 0.0248 |
| 0.92 | 61 | 118 | 3 | 86 | 66.8 | 41.5 | 97.5 | 4.7 | 42.2 | 95.3 | 57.8 | 65 | 93.8 | 97.5 | 41.7 | 0.0248 |
| 0.93 | 57 | 119 | 2 | 90 | 65.7 | 38.8 | 98.3 | 3.4 | 43.1 | 96.6 | 56.9 | 72.3 | 93.5 | 98.2 | 40.8 | 0.0165 |
| 0.94 | 55 | 119 | 2 | 92 | 64.9 | 37.4 | 98.3 | 3.5 | 43.6 | 96.5 | 56.4 | 71.6 | 93.4 | 98.1 | 40.2 | 0.0165 |
| 0.95 | 51 | 119 | 2 | 96 | 63.4 | 34.7 | 98.3 | 3.8 | 44.7 | 96.2 | 55.3 | 70 | 93.1 | 98 | 39.2 | 0.0165 |
| 0.96 | 45 | 119 | 2 | 102 | 61.2 | 30.6 | 98.3 | 4.3 | 46.2 | 95.7 | 53.8 | 67.3 | 92.7 | 97.7 | 37.8 | 0.0165 |
| 0.97 | 41 | 121 | 0 | 106 | 60.4 | 27.9 | 100 | 0 | 46.7 | 100 | 53.3 | 100 | 92.6 | 100 | 37.3 | 0 |
| 0.98 | 37 | 121 | 0 | 110 | 59 | 25.2 | 100 | 0 | 47.6 | 100 | 52.4 | 100 | 92.3 | 100 | 36.4 | 0 |
| 0.99 | 29 | 121 | 0 | 118 | 56 | 19.7 | 100 | 0 | 49.4 | 100 | 50.6 | 100 | 91.8 | 100 | 34.8 | 0 |
| 1 | 0 | 121 | 0 | 147 | 45.1 | 0 | 100 | — | 54.9 | — | 45.1 | — | 90 | — | 30 | 0 |

Table 10: Analysis of Sample Sets using Six Variable Model

TABLE 10A
Analysis of Training Set using Six Variable Model; n = 181, stage 1 = 96 stage 0 = 85; Stage 1 (evt) vs. Stage 0 (non-evt).

| | | | | | | | | | | Prevalence | | | | | | | | |
|---|---|---|---|---|---|---|---|---|---|---|---|---|---|---|---|---|---|---|
| | correct | | incorrect | | % | | | | | 53.0% | | 10% | | 70% | | | | |
| prob | evt | n-e | evt | n-e | accuracy | sensi | spec | f-pos | f-neg | PPV | NPV | PPV | NPV | PPV | NPV | 1-Spec | Sensi | AUC |
| 0 | 96 | 0 | 85 | 0 | 53 | 100 | 0 | 47 | — | 53 | — | 10 | — | 70 | — | 1 | 1 | |
| 0.01 | 96 | 0 | 85 | 0 | 53 | 100 | 0 | 47 | — | 53 | — | 10 | — | 70 | — | 1 | 1 | 0 |
| 0.02 | 96 | 0 | 85 | 0 | 53 | 100 | 0 | 47 | — | 53 | — | 10 | — | 70 | — | 1 | 1 | 0 |
| 0.03 | 95 | 5 | 80 | 1 | 55.2 | 99 | 5.9 | 45.7 | 16.7 | 54.3 | 83.3 | 10.5 | 98.1 | 71 | 70.8 | 0.9412 | 0.99 | 0.05852 |
| 0.04 | 94 | 6 | 79 | 2 | 55.2 | 97.9 | 7.1 | 45.7 | 25 | 54.3 | 75 | 10.5 | 96.8 | 71.1 | 59.2 | 0.9294 | 0.979 | 0.01158 |
| 0.05 | 94 | 9 | 76 | 2 | 56.9 | 97.9 | 10.6 | 44.7 | 18.2 | 55.3 | 81.8 | 10.8 | 97.9 | 71.9 | 68.5 | 0.8941 | 0.979 | 0.03456 |
| 0.06 | 94 | 12 | 73 | 2 | 58.6 | 97.9 | 14.1 | 43.7 | 14.3 | 56.3 | 85.7 | 11.2 | 98.4 | 72.7 | 74.4 | 0.8588 | 0.979 | 0.03456 |
| 0.07 | 94 | 12 | 73 | 2 | 58.6 | 97.9 | 14.1 | 43.7 | 14.3 | 56.3 | 85.7 | 11.2 | 98.4 | 72.7 | 74.4 | 0.8588 | 0.979 | 0 |
| 0.08 | 94 | 14 | 71 | 2 | 59.7 | 97.9 | 16.5 | 43 | 12.5 | 57 | 87.5 | 11.5 | 98.6 | 73.2 | 77.2 | 0.8353 | 0.979 | 0.02304 |
| 0.09 | 94 | 16 | 69 | 2 | 60.8 | 97.9 | 18.8 | 42.3 | 11.1 | 57.7 | 88.9 | 11.8 | 98.8 | 73.8 | 79.5 | 0.8118 | 0.979 | 0.02304 |
| 0.1 | 94 | 18 | 67 | 2 | 61.9 | 97.9 | 21.2 | 41.6 | 10 | 58.4 | 90 | 12.1 | 98.9 | 74.3 | 81.3 | 0.7882 | 0.979 | 0.02304 |
| 0.11 | 94 | 18 | 67 | 2 | 61.9 | 97.9 | 21.2 | 41.6 | 10 | 58.4 | 90 | 12.1 | 98.9 | 74.3 | 81.3 | 0.7882 | 0.979 | 0 |
| 0.12 | 94 | 22 | 63 | 2 | 64.1 | 97.9 | 25.9 | 40.1 | 8.3 | 59.9 | 91.7 | 12.8 | 99.1 | 75.5 | 84.2 | 0.7412 | 0.979 | 0.04608 |
| 0.13 | 93 | 25 | 60 | 3 | 65.2 | 96.9 | 29.4 | 39.2 | 10.7 | 60.8 | 89.3 | 13.2 | 98.8 | 76.2 | 80.1 | 0.7059 | 0.969 | 0.03438 |
| 0.14 | 93 | 26 | 59 | 3 | 65.7 | 96.9 | 30.6 | 38.8 | 10.3 | 61.2 | 89.7 | 13.4 | 98.9 | 76.5 | 80.8 | 0.6941 | 0.969 | 0.0114 |
| 0.15 | 93 | 30 | 55 | 3 | 68 | 96.9 | 35.3 | 37.2 | 9.1 | 62.8 | 90.9 | 14.3 | 99 | 77.7 | 82.9 | 0.6471 | 0.969 | 0.04559 |
| 0.16 | 93 | 33 | 52 | 3 | 69.6 | 96.9 | 38.8 | 35.9 | 8.3 | 64.1 | 91.7 | 15 | 99.1 | 78.7 | 84.2 | 0.6118 | 0.969 | 0.03419 |
| 0.17 | 92 | 35 | 50 | 4 | 70.2 | 95.8 | 41.2 | 35.2 | 10.3 | 64.8 | 89.7 | 15.3 | 98.9 | 79.2 | 80.9 | 0.5882 | 0.958 | 0.02267 |
| 0.18 | 92 | 39 | 46 | 4 | 72.4 | 95.8 | 45.9 | 33.3 | 9.3 | 66.7 | 90.7 | 16.4 | 99 | 80.5 | 82.5 | 0.5412 | 0.958 | 0.0451 |
| 0.19 | 92 | 40 | 45 | 4 | 72.9 | 95.8 | 47.1 | 32.8 | 9.1 | 67.2 | 90.9 | 16.7 | 99 | 80.9 | 82.9 | 0.5294 | 0.958 | 0.01128 |
| 0.2 | 92 | 42 | 43 | 4 | 74 | 95.8 | 49.4 | 31.9 | 8.7 | 68.1 | 91.3 | 17.4 | 99.1 | 81.6 | 83.6 | 0.5059 | 0.958 | 0.02255 |
| 0.21 | 92 | 44 | 41 | 4 | 75.1 | 95.8 | 51.8 | 30.8 | 8.3 | 69.2 | 91.7 | 18.1 | 99.1 | 82.3 | 84.2 | 0.4824 | 0.958 | 0.02255 |
| 0.22 | 92 | 45 | 40 | 4 | 75.7 | 95.8 | 52.9 | 30.3 | 8.2 | 69.7 | 91.8 | 18.5 | 99.1 | 82.6 | 84.5 | 0.4706 | 0.958 | 0.01128 |
| 0.23 | 92 | 46 | 39 | 4 | 76.2 | 95.8 | 54.1 | 29.8 | 8 | 70.2 | 92 | 18.8 | 99.2 | 83 | 84.8 | 0.4588 | 0.958 | 0.01128 |
| 0.24 | 92 | 47 | 38 | 4 | 76.8 | 95.8 | 55.3 | 29.2 | 7.8 | 70.8 | 92.2 | 19.2 | 99.2 | 83.3 | 85 | 0.4471 | 0.958 | 0.01128 |
| 0.25 | 92 | 48 | 37 | 4 | 77.3 | 95.8 | 56.5 | 28.7 | 7.7 | 71.3 | 92.3 | 19.7 | 99.2 | 83.7 | 85.3 | 0.4353 | 0.958 | 0.01128 |
| 0.26 | 91 | 50 | 35 | 5 | 77.9 | 94.8 | 58.8 | 27.8 | 9.1 | 72.2 | 90.9 | 20.4 | 99 | 84.3 | 82.9 | 0.4118 | 0.948 | 0.02243 |
| 0.27 | 91 | 51 | 34 | 5 | 78.5 | 94.8 | 60 | 27.2 | 8.9 | 72.8 | 91.1 | 20.8 | 99 | 84.7 | 83.2 | 0.4 | 0.948 | 0.01115 |
| 0.28 | 90 | 51 | 34 | 6 | 77.9 | 93.8 | 60 | 27.4 | 10.5 | 72.6 | 89.5 | 20.7 | 98.9 | 84.5 | 80.4 | 0.4 | 0.938 | 0 |
| 0.29 | 88 | 51 | 34 | 8 | 76.8 | 91.7 | 60 | 27.9 | 13.6 | 72.1 | 86.4 | 20.3 | 98.5 | 84.2 | 75.5 | 0.4 | 0.917 | 0 |
| 0.3 | 88 | 52 | 33 | 8 | 77.3 | 91.7 | 61.2 | 27.3 | 13.3 | 72.7 | 86.7 | 20.8 | 98.5 | 84.6 | 75.9 | 0.3882 | 0.917 | 0.01078 |
| 0.31 | 88 | 52 | 33 | 8 | 77.3 | 91.7 | 61.2 | 27.3 | 13.3 | 72.7 | 86.7 | 20.8 | 98.5 | 84.6 | 75.9 | 0.3882 | 0.917 | 0 |
| 0.32 | 88 | 54 | 31 | 8 | 78.5 | 91.7 | 63.5 | 26.1 | 12.9 | 73.9 | 87.1 | 21.8 | 98.6 | 85.4 | 76.6 | 0.3647 | 0.917 | 0.02157 |
| 0.33 | 87 | 56 | 29 | 9 | 79 | 90.6 | 65.9 | 25 | 13.8 | 75 | 86.2 | 22.8 | 98.4 | 86.1 | 75.1 | 0.3412 | 0.906 | 0.02145 |
| 0.34 | 87 | 57 | 28 | 9 | 79.6 | 90.6 | 67.1 | 24.3 | 13.6 | 75.7 | 86.4 | 23.4 | 98.5 | 86.5 | 75.4 | 0.3294 | 0.906 | 0.01066 |
| 0.35 | 87 | 60 | 25 | 9 | 81.2 | 90.6 | 70.6 | 22.3 | 13 | 77.7 | 87 | 25.5 | 98.5 | 87.8 | 76.3 | 0.2941 | 0.906 | 0.03199 |
| 0.36 | 87 | 62 | 23 | 9 | 82.3 | 90.6 | 72.9 | 20.9 | 12.7 | 79.1 | 87.3 | 27.1 | 98.6 | 88.7 | 76.9 | 0.2706 | 0.906 | 0.02132 |
| 0.37 | 87 | 62 | 23 | 9 | 82.3 | 90.6 | 72.9 | 20.9 | 12.7 | 79.1 | 87.3 | 27.1 | 98.6 | 88.7 | 76.9 | 0.2706 | 0.906 | 0 |
| 0.38 | 86 | 63 | 22 | 10 | 82.3 | 89.6 | 74.1 | 20.4 | 13.7 | 79.6 | 86.3 | 27.8 | 98.5 | 89 | 75.3 | 0.2588 | 0.896 | 0.0106 |
| 0.39 | 85 | 63 | 22 | 11 | 81.8 | 88.5 | 74.1 | 20.6 | 14.9 | 79.4 | 85.1 | 27.5 | 98.3 | 88.9 | 73.5 | 0.2588 | 0.885 | 0 |
| 0.4 | 85 | 63 | 22 | 11 | 81.8 | 88.5 | 74.1 | 20.6 | 14.9 | 79.4 | 85.1 | 27.5 | 98.3 | 88.9 | 73.5 | 0.2588 | 0.885 | 0 |
| 0.41 | 85 | 63 | 22 | 11 | 81.8 | 88.5 | 74.1 | 20.6 | 14.9 | 79.4 | 85.1 | 27.5 | 98.3 | 88.9 | 73.5 | 0.2588 | 0.885 | 0 |
| 0.42 | 84 | 63 | 22 | 12 | 81.2 | 87.5 | 74.1 | 20.8 | 16 | 79.2 | 84 | 27.3 | 98.2 | 88.7 | 71.8 | 0.2588 | 0.875 | 0 |
| 0.43 | 84 | 64 | 21 | 12 | 81.8 | 87.5 | 75.3 | 20 | 15.8 | 80 | 84.2 | 28.2 | 98.2 | 89.2 | 72.1 | 0.2471 | 0.875 | 0.01029 |
| 0.44 | 84 | 64 | 21 | 12 | 81.8 | 87.5 | 75.3 | 20 | 15.8 | 80 | 84.2 | 28.2 | 98.2 | 89.2 | 72.1 | 0.2471 | 0.875 | 0 |
| 0.45 | 84 | 64 | 21 | 12 | 81.8 | 87.5 | 75.3 | 20 | 15.8 | 80 | 84.2 | 28.2 | 98.2 | 89.2 | 72.1 | 0.2471 | 0.875 | 0 |
| 0.46 | 82 | 66 | 19 | 14 | 81.8 | 85.4 | 77.6 | 18.8 | 17.5 | 81.2 | 82.5 | 29.8 | 98 | 89.9 | 69.5 | 0.2235 | 0.854 | 0.02034 |
| 0.47 | 80 | 68 | 17 | 16 | 81.8 | 83.3 | 80 | 17.5 | 19 | 82.5 | 81 | 31.6 | 97.7 | 90.7 | 67.3 | 0.2 | 0.833 | 0.01985 |
| 0.48 | 78 | 69 | 16 | 18 | 81.2 | 81.3 | 81.2 | 17 | 20.7 | 83 | 79.3 | 32.4 | 97.5 | 91 | 65 | 0.1882 | 0.813 | 0.00968 |
| 0.49 | 78 | 70 | 15 | 18 | 81.8 | 81.3 | 82.4 | 16.1 | 20.5 | 83.9 | 79.5 | 33.8 | 97.5 | 91.5 | 65.3 | 0.1765 | 0.813 | 0.00956 |
| 0.5 | 78 | 70 | 15 | 18 | 81.8 | 81.3 | 82.4 | 16.1 | 20.5 | 83.9 | 79.5 | 33.8 | 97.5 | 91.5 | 65.3 | 0.1765 | 0.813 | 0 |
| 0.51 | 77 | 70 | 15 | 19 | 81.2 | 80.2 | 82.4 | 16.3 | 21.3 | 83.7 | 78.7 | 33.6 | 97.4 | 91.4 | 64.1 | 0.1765 | 0.802 | 0 |
| 0.52 | 77 | 70 | 15 | 19 | 81.2 | 80.2 | 82.4 | 16.3 | 21.3 | 83.7 | 78.7 | 33.6 | 97.4 | 91.4 | 64.1 | 0.1765 | 0.802 | 0 |
| 0.53 | 76 | 70 | 15 | 20 | 80.7 | 79.2 | 82.4 | 16.5 | 22.2 | 83.5 | 77.8 | 33.3 | 97.3 | 91.3 | 62.9 | 0.1765 | 0.792 | 0 |
| 0.54 | 76 | 71 | 14 | 20 | 81.2 | 79.2 | 83.5 | 15.6 | 22 | 84.4 | 78 | 34.8 | 97.3 | 91.8 | 63.2 | 0.1647 | 0.792 | 0.00931 |
| 0.55 | 76 | 73 | 12 | 20 | 82.3 | 79.2 | 85.9 | 13.6 | 21.5 | 86.4 | 78.5 | 38.4 | 97.4 | 92.9 | 63.9 | 0.1412 | 0.792 | 0.01863 |
| 0.56 | 75 | 74 | 11 | 21 | 82.3 | 78.1 | 87.1 | 12.8 | 22.1 | 87.2 | 77.9 | 40.1 | 97.3 | 93.4 | 63 | 0.1294 | 0.781 | 0.00925 |
| 0.57 | 74 | 75 | 10 | 22 | 82.3 | 77.1 | 88.2 | 11.9 | 22.7 | 88.1 | 77.3 | 42.1 | 97.2 | 93.9 | 62.3 | 0.1176 | 0.771 | 0.00913 |
| 0.58 | 72 | 75 | 10 | 24 | 81.2 | 75 | 88.2 | 12.2 | 24.2 | 87.8 | 75.8 | 41.5 | 96.9 | 93.7 | 60.2 | 0.1176 | 0.75 | 0 |
| 0.59 | 72 | 75 | 10 | 24 | 81.2 | 75 | 88.2 | 12.2 | 24.2 | 87.8 | 75.8 | 41.5 | 96.9 | 93.7 | 60.2 | 0.1176 | 0.75 | 0 |
| 0.6 | 72 | 75 | 10 | 24 | 81.2 | 75 | 88.2 | 12.2 | 24.2 | 87.8 | 75.8 | 41.5 | 96.9 | 93.7 | 60.2 | 0.1176 | 0.75 | 0 |
| 0.61 | 71 | 75 | 10 | 25 | 80.7 | 74 | 88.2 | 12.3 | 25 | 87.7 | 75 | 41.1 | 96.8 | 93.6 | 59.2 | 0.1176 | 0.74 | 0 |
| 0.62 | 66 | 76 | 9 | 30 | 78.5 | 68.8 | 89.4 | 12 | 28.3 | 88 | 71.7 | 41.9 | 96.3 | 93.8 | 55.1 | 0.1059 | 0.688 | 0.0084 |
| 0.63 | 66 | 77 | 8 | 30 | 79 | 68.8 | 90.6 | 10.8 | 28 | 89.2 | 72 | 44.8 | 96.3 | 94.5 | 55.4 | 0.0941 | 0.688 | 0.00809 |
| 0.64 | 66 | 77 | 8 | 30 | 79 | 68.8 | 90.6 | 10.8 | 28 | 89.2 | 72 | 44.8 | 96.3 | 94.5 | 55.4 | 0.0941 | 0.688 | 0 |
| 0.65 | 65 | 77 | 8 | 31 | 78.5 | 67.7 | 90.6 | 11 | 28.7 | 89 | 71.3 | 44.4 | 96.2 | 94.4 | 54.6 | 0.0941 | 0.677 | 0 |
| 0.66 | 63 | 77 | 8 | 33 | 77.3 | 65.6 | 90.6 | 11.3 | 30 | 88.7 | 70 | 43.7 | 96 | 94.2 | 53 | 0.0941 | 0.656 | 0 |
| 0.67 | 63 | 79 | 6 | 33 | 78.5 | 65.6 | 92.9 | 8.7 | 29.5 | 91.3 | 70.5 | 50.8 | 96.1 | 95.6 | 53.7 | 0.0706 | 0.656 | 0.01544 |
| 0.68 | 62 | 79 | 6 | 34 | 77.9 | 64.6 | 92.9 | 8.8 | 30.1 | 91.2 | 69.9 | 50.4 | 95.9 | 95.5 | 52.9 | 0.0706 | 0.646 | 0 |
| 0.69 | 61 | 80 | 5 | 35 | 77.9 | 63.5 | 94.1 | 7.6 | 30.4 | 92.4 | 69.6 | 54.6 | 95.9 | 96.2 | 52.5 | 0.0588 | 0.635 | 0.00754 |
| 0.7 | 58 | 80 | 5 | 38 | 76.2 | 60.4 | 94.1 | 7.9 | 32.2 | 92.1 | 67.8 | 53.3 | 95.5 | 96 | 50.5 | 0.0588 | 0.604 | 0 |
| 0.71 | 58 | 80 | 5 | 38 | 76.2 | 60.4 | 94.1 | 7.9 | 32.2 | 92.1 | 67.8 | 53.3 | 95.5 | 96 | 50.5 | 0.0588 | 0.604 | 0 |
| 0.72 | 58 | 80 | 5 | 38 | 76.2 | 60.4 | 94.1 | 7.9 | 32.2 | 92.1 | 67.8 | 53.3 | 95.5 | 96 | 50.5 | 0.0588 | 0.604 | 0 |
| 0.73 | 58 | 80 | 5 | 38 | 76.2 | 60.4 | 94.1 | 7.9 | 32.2 | 92.1 | 67.8 | 53.3 | 95.5 | 96 | 50.5 | 0.0588 | 0.604 | 0 |
| 0.74 | 57 | 80 | 5 | 39 | 75.7 | 59.4 | 94.1 | 8.1 | 32.8 | 91.9 | 67.2 | 52.9 | 95.4 | 95.9 | 49.8 | 0.0588 | 0.594 | 0 |
| 0.75 | 56 | 80 | 5 | 40 | 75.1 | 58.3 | 94.1 | 8.2 | 33.3 | 91.8 | 66.7 | 52.4 | 95.3 | 95.9 | 49.2 | 0.0588 | 0.583 | 0 |
| 0.76 | 56 | 80 | 5 | 40 | 75.1 | 58.3 | 94.1 | 8.2 | 33.3 | 91.8 | 66.7 | 52.4 | 95.3 | 95.9 | 49.2 | 0.0588 | 0.583 | 0 |
| 0.77 | 54 | 82 | 3 | 42 | 75.1 | 56.3 | 96.5 | 5.3 | 33.9 | 94.7 | 66.1 | 63.9 | 95.2 | 97.4 | 48.6 | 0.0353 | 0.563 | 0.01348 |
| 0.78 | 52 | 82 | 3 | 44 | 74 | 54.2 | 96.5 | 5.5 | 34.9 | 94.5 | 65.1 | 63 | 95 | 97.3 | 47.4 | 0.0353 | 0.542 | 0 |
| 0.79 | 52 | 82 | 3 | 44 | 74 | 54.2 | 96.5 | 5.5 | 34.9 | 94.5 | 65.1 | 63 | 95 | 97.3 | 47.4 | 0.0353 | 0.542 | 0 |
| 0.8 | 50 | 82 | 3 | 46 | 72.9 | 52.1 | 96.5 | 5.7 | 35.9 | 94.3 | 64.1 | 62.1 | 94.8 | 97.2 | 46.3 | 0.0353 | 0.521 | 0 |
| 0.81 | 49 | 82 | 3 | 47 | 72.4 | 51 | 96.5 | 5.8 | 36.4 | 94.2 | 63.6 | 61.6 | 94.7 | 97.1 | 45.8 | 0.0353 | 0.51 | 0 |
| 0.82 | 49 | 83 | 2 | 47 | 72.9 | 51 | 97.6 | 3.9 | 36.2 | 96.1 | 63.8 | 70.7 | 94.7 | 98.1 | 46.1 | 0.0235 | 0.51 | 0.00601 |
| 0.83 | 49 | 83 | 2 | 47 | 72.9 | 51 | 97.6 | 3.9 | 36.2 | 96.1 | 63.8 | 70.7 | 94.7 | 98.1 | 46.1 | 0.0235 | 0.51 | 0 |
| 0.84 | 48 | 83 | 2 | 48 | 72.4 | 50 | 97.6 | 4 | 36.6 | 96 | 63.4 | 70.2 | 94.6 | 98 | 45.6 | 0.0235 | 0.5 | 0 |
| 0.85 | 48 | 83 | 2 | 48 | 72.4 | 50 | 97.6 | 4 | 36.6 | 96 | 63.4 | 70.2 | 94.6 | 98 | 45.6 | 0.0235 | 0.5 | 0 |
| 0.86 | 47 | 83 | 2 | 49 | 71.8 | 49 | 97.6 | 4.1 | 37.1 | 95.9 | 62.9 | 69.8 | 94.5 | 98 | 45.1 | 0.0235 | 0.49 | 0 |
| 0.87 | 45 | 83 | 2 | 51 | 70.7 | 46.9 | 97.6 | 4.3 | 38.1 | 95.7 | 61.9 | 68.9 | 94.3 | 97.9 | 44.1 | 0.0235 | 0.469 | 0 |
| 0.88 | 41 | 83 | 2 | 55 | 68.5 | 42.7 | 97.6 | 4.7 | 39.9 | 95.3 | 60.1 | 66.9 | 93.9 | 97.7 | 42.2 | 0.0235 | 0.427 | 0 |
| 0.89 | 40 | 83 | 2 | 56 | 68 | 41.7 | 97.6 | 4.8 | 40.3 | 95.2 | 59.7 | 66.3 | 93.8 | 97.6 | 41.8 | 0.0235 | 0.417 | 0 |
| 0.9 | 39 | 83 | 2 | 57 | 67.4 | 40.6 | 97.6 | 4.9 | 40.7 | 95.1 | 59.3 | 65.7 | 93.7 | 97.6 | 41.3 | 0.0235 | 0.406 | 0 |
| 0.91 | 39 | 83 | 2 | 57 | 67.4 | 40.6 | 97.6 | 4.9 | 40.7 | 95.1 | 59.3 | 65.7 | 93.7 | 97.6 | 41.3 | 0.0235 | 0.406 | 0 |
| 0.92 | 37 | 83 | 2 | 59 | 66.3 | 38.5 | 97.6 | 5.1 | 41.5 | 94.9 | 58.5 | 64.5 | 93.5 | 97.5 | 40.5 | 0.0235 | 0.385 | 0 |
| 0.93 | 36 | 83 | 2 | 60 | 65.7 | 37.5 | 97.6 | 5.3 | 42 | 94.7 | 58 | 63.9 | 93.4 | 97.4 | 40.1 | 0.0235 | 0.375 | 0 |
| 0.94 | 34 | 84 | 1 | 62 | 65.2 | 35.4 | 98.8 | 2.9 | 42.5 | 97.1 | 57.5 | 77 | 93.2 | 98.6 | 39.6 | 0.0118 | 0.354 | 0.00429 |
| 0.95 | 33 | 84 | 1 | 63 | 64.6 | 34.4 | 98.8 | 2.9 | 42.9 | 97.1 | 57.1 | 76.5 | 93.1 | 98.6 | 39.2 | 0.0118 | 0.344 | 0 |
| 0.96 | 27 | 84 | 1 | 69 | 61.3 | 28.1 | 98.8 | 3.6 | 45.1 | 96.4 | 54.9 | 72.6 | 92.5 | 98.2 | 37.1 | 0.0118 | 0.281 | 0 |
| 0.97 | 25 | 84 | 1 | 71 | 60.2 | 26 | 98.8 | 3.8 | 45.8 | 96.2 | 54.2 | 71.1 | 92.3 | 98.1 | 36.4 | 0.0118 | 0.26 | 0 |
| 0.98 | 21 | 85 | 0 | 75 | 58.6 | 21.9 | 100 | 0 | 46.9 | 100 | 53.1 | 100 | 92 | 100 | 35.4 | 0 | 0.219 | 0.00282 |
| 0.99 | 16 | 85 | 0 | 80 | 55.8 | 16.7 | 100 | 0 | 48.5 | 100 | 51.5 | 100 | 91.5 | 100 | 34 | 0 | 0.167 | 0 |
| 1 | 0 | 85 | 0 | 96 | 47 | 0 | 100 | — | 53 | — | 47 | — | 90 | — | 30 | 0 | 0 | 0 |
| | | | | | | | | | | | | | | | | | AUC | 0.89326 |

TABLE 10B
Analysis of Validation (Testing) Set using Six Variable Model; n = 187, stage 1 = 89 stage 0 = 98; Stage 1 (evt) vs. Stage 0 (non-evt).

| | | | | | | | | | | Prevalence | | | | | | | | |
|---|---|---|---|---|---|---|---|---|---|---|---|---|---|---|---|---|---|---|
| | correct | | incorrect | | % | | | | | 47.6% | | 10% | | 70% | | | | |
| prob | evt | n-e | evt | n-e | accuracy | sensi | spec | f-pos | f-neg | PPV | NPV | PPV | NPV | PPV | NPV | 1-Spec | Sensi | AUC |
| 0 | 89 | 0 | 98 | 0 | 47.6 | 100 | 0 | 52.4 | — | 47.6 | — | 10 | — | 70 | — | 1 | 1 | |
| 0.01 | 89 | 0 | 98 | 0 | 47.6 | 100 | 0 | 52.4 | — | 47.6 | — | 10 | — | 70 | — | 1 | 1 | 0 |
| 0.02 | 89 | 1 | 97 | 0 | 48.1 | 100 | 1 | 52.2 | 0 | 47.8 | 100 | 10.1 | 100 | 70.2 | 100 | 0.9898 | 1 | 0.010204 |
| 0.03 | 89 | 3 | 95 | 0 | 49.2 | 100 | 3.1 | 51.6 | 0 | 48.4 | 100 | 10.3 | 100 | 70.6 | 100 | 0.9694 | 1 | 0.020408 |
| 0.04 | 89 | 6 | 92 | 0 | 50.8 | 100 | 6.1 | 50.8 | 0 | 49.2 | 100 | 10.6 | 100 | 71.3 | 100 | 0.9388 | 1 | 0.030612 |
| 0.05 | 89 | 10 | 88 | 0 | 52.9 | 100 | 10.2 | 49.7 | 0 | 50.3 | 100 | 11 | 100 | 72.2 | 100 | 0.898 | 1 | 0.040816 |
| 0.06 | 89 | 13 | 85 | 0 | 54.5 | 100 | 13.3 | 48.9 | 0 | 51.1 | 100 | 11.4 | 100 | 72.9 | 100 | 0.8673 | 1 | 0.630612 |
| 0.07 | 89 | 17 | 81 | 0 | 56.7 | 100 | 17.3 | 47.6 | 0 | 52.4 | 100 | 11.9 | 100 | 73.8 | 100 | 0.8265 | 1 | 0.040816 |
| 0.08 | 89 | 20 | 78 | 0 | 58.3 | 100 | 20.4 | 46.7 | 0 | 53.3 | 100 | 12.3 | 100 | 74.6 | 100 | 0.7959 | 1 | 0.030612 |
| 0.09 | 89 | 20 | 78 | 0 | 58.3 | 100 | 20.4 | 46.7 | 0 | 53.3 | 100 | 12.3 | 100 | 74.6 | 100 | 0.7959 | 1 | 0 |
| 0.1 | 89 | 22 | 76 | 0 | 59.4 | 100 | 22.4 | 46.1 | 0 | 53.9 | 100 | 12.5 | 100 | 75.1 | 100 | 0.7755 | 1 | 0.020408 |
| 0.11 | 89 | 23 | 75 | 0 | 59.9 | 100 | 23.5 | 45.7 | 0 | 54.3 | 100 | 12.7 | 100 | 75.3 | 100 | 0.7653 | 1 | 0.010204 |
| 0.12 | 89 | 24 | 74 | 0 | 60.4 | 100 | 24.5 | 45.4 | 0 | 54.6 | 100 | 12.8 | 100 | 75.6 | 100 | 0.7551 | 1 | 0.010204 |
| 0.13 | 89 | 26 | 72 | 0 | 61.5 | 100 | 26.5 | 44.7 | 0 | 55.3 | 100 | 13.1 | 100 | 76.1 | 100 | 0.7347 | 1 | 0.020408 |
| 0.14 | 88 | 28 | 70 | 1 | 62 | 98.9 | 28.6 | 44.3 | 3.4 | 55.7 | 96.6 | 13.3 | 99.6 | 76.4 | 91.6 | 0.7143 | 0.989 | 0.020294 |
| 0.15 | 88 | 31 | 67 | 1 | 63.6 | 98.9 | 31.6 | 43.2 | 3.1 | 56.8 | 96.9 | 13.8 | 99.6 | 77.1 | 92.3 | 0.6837 | 0.989 | 0.030268 |
| 0.16 | 88 | 35 | 63 | 1 | 65.8 | 98.9 | 35.7 | 41.7 | 2.8 | 58.3 | 97.2 | 14.6 | 99.7 | 78.2 | 93.2 | 0.6429 | 0.989 | 0.040358 |
| 0.17 | 86 | 37 | 61 | 3 | 65.8 | 96.6 | 37.8 | 41.5 | 7.5 | 58.5 | 92.5 | 14.7 | 99 | 78.4 | 82.8 | 0.6224 | 0.966 | 0.01995 |
| 0.18 | 86 | 38 | 60 | 3 | 66.3 | 96.6 | 38.8 | 41.1 | 7.3 | 58.9 | 92.7 | 14.9 | 99 | 78.6 | 83.1 | 0.6122 | 0.966 | 0.00986 |
| 0.19 | 86 | 41 | 57 | 3 | 67.9 | 96.6 | 41.8 | 39.9 | 6.8 | 60.1 | 93.2 | 15.6 | 99.1 | 79.5 | 84.2 | 0.5816 | 0.966 | 0.02958 |
| 0.2 | 86 | 44 | 54 | 3 | 69.5 | 96.6 | 44.9 | 38.6 | 6.4 | 61.4 | 93.6 | 16.3 | 99.2 | 80.4 | 85.1 | 0.551 | 0.966 | 0.02958 |
| 0.21 | 83 | 47 | 51 | 6 | 69.5 | 93.3 | 48 | 38.1 | 11.3 | 61.9 | 88.7 | 16.6 | 98.5 | 80.7 | 75.3 | 0.5204 | 0.933 | 0.029064 |
| 0.22 | 83 | 51 | 47 | 6 | 71.7 | 93.3 | 52 | 36.2 | 10.5 | 63.8 | 89.5 | 17.8 | 98.6 | 81.9 | 76.8 | 0.4796 | 0.933 | 0.038065 |
| 0.23 | 81 | 54 | 44 | 8 | 72.2 | 91 | 55.1 | 35.2 | 12.9 | 64.8 | 87.1 | 18.4 | 98.2 | 82.5 | 72.4 | 0.449 | 0.91 | 0.028205 |
| 0.24 | 81 | 56 | 42 | 8 | 73.3 | 91 | 57.1 | 34.1 | 12.5 | 65.9 | 87.5 | 19.1 | 98.3 | 83.2 | 73.2 | 0.4286 | 0.91 | 0.018574 |
| 0.25 | 81 | 56 | 42 | 8 | 73.3 | 91 | 57.1 | 34.1 | 12.5 | 65.9 | 87.5 | 19.1 | 98.3 | 83.2 | 73.2 | 0.4286 | 0.91 | 0 |
| 0.26 | 81 | 58 | 40 | 8 | 74.3 | 91 | 59.2 | 33.1 | 12.1 | 66.9 | 87.9 | 19.9 | 98.3 | 83.9 | 73.8 | 0.4082 | 0.91 | 0.018574 |
| 0.27 | 80 | 61 | 37 | 9 | 75.4 | 89.9 | 62.2 | 31.6 | 12.9 | 68.4 | 87.1 | 20.9 | 98.2 | 84.7 | 72.5 | 0.3776 | 0.899 | 0.027689 |
| 0.28 | 80 | 63 | 35 | 9 | 76.5 | 89.9 | 64.3 | 30.4 | 12.5 | 69.6 | 87.5 | 21.9 | 98.3 | 85.4 | 73.2 | 0.3571 | 0.899 | 0.018344 |
| 0.29 | 79 | 63 | 35 | 10 | 75.9 | 88.8 | 64.3 | 30.7 | 13.7 | 69.3 | 86.3 | 21.6 | 98.1 | 85.3 | 71 | 0.3571 | 0.888 | 0 |
| 0.3 | 79 | 63 | 35 | 10 | 75.9 | 88.8 | 64.3 | 30.7 | 13.7 | 69.3 | 86.3 | 21.6 | 98.1 | 85.3 | 71 | 0.3571 | 0.888 | 0 |
| 0.31 | 79 | 63 | 35 | 10 | 75.9 | 88.8 | 64.3 | 30.7 | 13.7 | 69.3 | 86.3 | 21.6 | 98.1 | 85.3 | 71 | 0.3571 | 0.888 | 0 |
| 0.32 | 79 | 65 | 33 | 10 | 77 | 88.8 | 66.3 | 29.5 | 13.3 | 70.5 | 86.7 | 22.7 | 98.2 | 86 | 71.7 | 0.3367 | 0.888 | 0.018115 |
| 0.33 | 79 | 66 | 32 | 10 | 77.5 | 88.8 | 67.3 | 28.8 | 13.2 | 71.2 | 86.8 | 23.2 | 98.2 | 86.4 | 72 | 0.3265 | 0.888 | 0.009058 |
| 0.34 | 79 | 67 | 31 | 10 | 78.1 | 88.8 | 68.4 | 28.2 | 13 | 71.8 | 87 | 23.8 | 98.2 | 86.8 | 72.3 | 0.3163 | 0.888 | 0.009058 |
| 0.35 | 79 | 67 | 31 | 10 | 78.1 | 88.8 | 68.4 | 28.2 | 13 | 71.8 | 87 | 23.8 | 98.2 | 86.8 | 72.3 | 0.3163 | 0.888 | 0 |
| 0.36 | 79 | 67 | 31 | 10 | 78.1 | 88.8 | 68.4 | 28.2 | 13 | 71.8 | 87 | 23.8 | 98.2 | 86.8 | 72.3 | 0.3163 | 0.888 | 0 |
| 0.37 | 78 | 68 | 30 | 11 | 78.1 | 87.6 | 69.4 | 27.8 | 13.9 | 72.2 | 86.1 | 24.1 | 98.1 | 87 | 70.6 | 0.3061 | 0.876 | 0.009 |
| 0.38 | 78 | 68 | 30 | 11 | 78.1 | 87.6 | 69.4 | 27.8 | 13.9 | 72.2 | 86.1 | 24.1 | 98.1 | 87 | 70.6 | 0.3061 | 0.876 | 0 |
| 0.39 | 77 | 70 | 28 | 12 | 78.6 | 86.5 | 71.4 | 26.7 | 14.6 | 73.3 | 85.4 | 25.2 | 97.9 | 87.6 | 69.4 | 0.2857 | 0.865 | 0.017771 |
| 0.4 | 77 | 70 | 28 | 12 | 78.6 | 86.5 | 71.4 | 26.7 | 14.6 | 73.3 | 85.4 | 25.2 | 97.9 | 87.6 | 69.4 | 0.2857 | 0.865 | 0 |
| 0.41 | 76 | 71 | 27 | 13 | 78.6 | 85.4 | 72.4 | 26.2 | 15.5 | 73.8 | 84.5 | 25.6 | 97.8 | 87.9 | 68 | 0.2755 | 0.854 | 0.008771 |
| 0.42 | 76 | 71 | 27 | 13 | 78.6 | 85.4 | 72.4 | 26.2 | 15.5 | 73.8 | 84.5 | 25.6 | 97.8 | 87.9 | 68 | 0.2755 | 0.854 | 0 |
| 0.43 | 73 | 72 | 26 | 16 | 77.5 | 82 | 73.5 | 26.3 | 18.2 | 73.7 | 81.8 | 25.6 | 97.4 | 87.8 | 63.7 | 0.2653 | 0.82 | 0.008542 |
| 0.44 | 73 | 72 | 26 | 16 | 77.5 | 82 | 73.5 | 26.3 | 18.2 | 73.7 | 81.8 | 25.6 | 97.4 | 87.8 | 63.7 | 0.2653 | 0.82 | 0 |
| 0.45 | 73 | 72 | 26 | 16 | 77.5 | 82 | 73.5 | 26.3 | 18.2 | 73.7 | 81.8 | 25.6 | 97.4 | 87.8 | 63.7 | 0.2653 | 0.82 | 0 |
| 0.46 | 72 | 73 | 25 | 17 | 77.5 | 80.9 | 74.5 | 25.8 | 18.9 | 74.2 | 81.1 | 26.1 | 97.2 | 88.1 | 62.6 | 0.2551 | 0.809 | 0.008312 |
| 0.47 | 71 | 74 | 24 | 18 | 77.5 | 79.8 | 75.5 | 25.3 | 19.6 | 74.7 | 80.4 | 26.6 | 97.1 | 88.4 | 61.5 | 0.2449 | 0.798 | 0.008198 |
| 0.48 | 70 | 74 | 24 | 19 | 77 | 78.7 | 75.5 | 25.5 | 20.4 | 74.5 | 79.6 | 26.3 | 97 | 88.2 | 60.3 | 0.2449 | 0.787 | 0 |
| 0.49 | 69 | 75 | 23 | 20 | 77 | 77.5 | 76.5 | 25 | 21.1 | 75 | 78.9 | 26.8 | 96.8 | 88.5 | 59.3 | 0.2347 | 0.775 | 0.007968 |
| 0.5 | 69 | 77 | 21 | 20 | 78.1 | 77.5 | 78.6 | 23.3 | 20.6 | 76.7 | 79.4 | 28.7 | 96.9 | 89.4 | 60 | 0.2143 | 0.775 | 0.015822 |
| 0.51 | 68 | 79 | 19 | 21 | 78.6 | 76.4 | 80.6 | 21.8 | 21 | 78.2 | 79 | 30.5 | 96.9 | 90.2 | 59.4 | 0.1939 | 0.764 | 0.015707 |
| 0.52 | 67 | 80 | 18 | 22 | 78.6 | 75.3 | 81.6 | 21.2 | 21.6 | 78.8 | 78.4 | 31.3 | 96.7 | 90.5 | 58.6 | 0.1837 | 0.753 | 0.007736 |
| 0.53 | 67 | 80 | 18 | 22 | 78.6 | 75.3 | 81.6 | 21.2 | 21.6 | 78.8 | 78.4 | 31.3 | 96.7 | 90.5 | 58.6 | 0.1837 | 0.753 | 0 |
| 0.54 | 67 | 81 | 17 | 22 | 79.1 | 75.3 | 82.7 | 20.2 | 21.4 | 79.8 | 78.6 | 32.5 | 96.8 | 91 | 58.9 | 0.1735 | 0.753 | 0.007682 |
| 0.55 | 66 | 82 | 16 | 23 | 79.1 | 74.2 | 83.7 | 19.5 | 21.9 | 80.5 | 78.1 | 33.5 | 96.7 | 91.4 | 58.1 | 0.1633 | 0.742 | 0.007624 |
| 0.56 | 65 | 82 | 16 | 24 | 78.6 | 73 | 83.7 | 19.8 | 22.6 | 80.2 | 77.4 | 33.2 | 96.5 | 91.3 | 57.1 | 0.1633 | 0.73 | 0 |
| 0.57 | 64 | 82 | 16 | 25 | 78.1 | 71.9 | 83.7 | 20 | 23.4 | 80 | 76.6 | 32.9 | 96.4 | 91.1 | 56.1 | 0.1633 | 0.719 | 0 |
| 0.58 | 64 | 84 | 14 | 25 | 79.1 | 71.9 | 85.7 | 17.9 | 22.9 | 82.1 | 77.1 | 35.9 | 96.5 | 92.2 | 56.7 | 0.1429 | 0.719 | 0.014676 |
| 0.59 | 63 | 84 | 14 | 26 | 78.6 | 70.8 | 85.7 | 18.2 | 23.6 | 81.8 | 76.4 | 35.5 | 96.4 | 92 | 55.7 | 0.1429 | 0.708 | 0 |
| 0.6 | 63 | 85 | 13 | 26 | 79.1 | 70.8 | 86.7 | 17.1 | 23.4 | 82.9 | 76.6 | 37.2 | 96.4 | 92.6 | 56 | 0.1327 | 0.708 | 0.007223 |
| 0.61 | 59 | 86 | 12 | 30 | 77.5 | 66.3 | 87.8 | 16.9 | 25.9 | 83.1 | 74.1 | 37.6 | 95.9 | 92.7 | 52.7 | 0.1224 | 0.663 | 0.006994 |
| 0.62 | 58 | 86 | 12 | 31 | 77 | 65.2 | 87.8 | 17.1 | 26.5 | 82.9 | 73.5 | 37.2 | 95.8 | 92.5 | 51.9 | 0.1224 | 0.652 | 0 |
| 0.63 | 58 | 86 | 12 | 31 | 77 | 65.2 | 87.8 | 17.1 | 26.5 | 82.9 | 73.5 | 37.2 | 95.8 | 92.5 | 51.9 | 0.1224 | 0.652 | 0 |
| 0.64 | 58 | 87 | 11 | 31 | 77.5 | 65.2 | 88.8 | 15.9 | 26.3 | 84.1 | 73.7 | 39.2 | 95.8 | 93.1 | 52.2 | 0.1122 | 0.652 | 0.00665 |
| 0.65 | 56 | 88 | 10 | 33 | 77 | 62.9 | 89.8 | 15.2 | 27.3 | 84.8 | 72.7 | 40.7 | 95.6 | 93.5 | 50.9 | 0.102 | 0.629 | 0.006535 |
| 0.66 | 55 | 89 | 9 | 34 | 77 | 61.8 | 90.8 | 14.1 | 27.6 | 85.9 | 72.4 | 42.8 | 95.5 | 94 | 50.5 | 0.0918 | 0.618 | 0.006363 |
| 0.67 | 53 | 89 | 9 | 36 | 75.9 | 59.6 | 90.8 | 14.5 | 28.8 | 85.5 | 71.2 | 41.9 | 95.3 | 93.8 | 49 | 0.0918 | 0.596 | 0 |
| 0.68 | 52 | 90 | 8 | 37 | 75.9 | 58.4 | 91.8 | 13.3 | 29.1 | 86.7 | 70.9 | 44.3 | 95.2 | 94.4 | 48.6 | 0.0816 | 0.584 | 0.006019 |
| 0.69 | 51 | 90 | 8 | 38 | 75.4 | 57.3 | 91.8 | 13.6 | 29.7 | 86.4 | 70.3 | 43.8 | 95.1 | 94.2 | 48 | 0.0816 | 0.573 | 0 |
| 0.7 | 51 | 90 | 8 | 38 | 75.4 | 57.3 | 91.8 | 13.6 | 29.7 | 86.4 | 70.3 | 43.8 | 95.1 | 94.2 | 48 | 0.0816 | 0.573 | 0 |
| 0.71 | 50 | 91 | 7 | 39 | 75.4 | 56.2 | 92.9 | 12.3 | 30 | 87.7 | 70 | 46.6 | 95 | 94.8 | 47.6 | 0.0714 | 0.562 | 0.00579 |
| 0.72 | 49 | 91 | 7 | 40 | 74.9 | 55.1 | 92.9 | 12.5 | 30.5 | 87.5 | 69.5 | 46.1 | 94.9 | 94.7 | 47 | 0.0714 | 0.551 | 0 |
| 0.73 | 48 | 91 | 7 | 41 | 74.3 | 53.9 | 92.9 | 12.7 | 31.1 | 87.3 | 68.9 | 45.6 | 94.8 | 94.6 | 46.3 | 0.0714 | 0.539 | 0 |
| 0.74 | 48 | 92 | 6 | 41 | 74.9 | 53.9 | 93.9 | 11.1 | 30.8 | 88.9 | 69.2 | 49.5 | 94.8 | 95.4 | 46.6 | 0.0612 | 0.539 | 0.005503 |
| 0.75 | 48 | 93 | 5 | 41 | 75.4 | 53.9 | 94.9 | 9.4 | 30.6 | 90.6 | 69.4 | 54 | 94.9 | 96.1 | 46.9 | 0.051 | 0.539 | 0.005503 |
| 0.76 | 48 | 93 | 5 | 41 | 75.4 | 53.9 | 94.9 | 9.4 | 30.6 | 90.6 | 69.4 | 54 | 94.9 | 96.1 | 46.9 | 0.051 | 0.539 | 0 |
| 0.77 | 48 | 93 | 5 | 41 | 75.4 | 53.9 | 94.9 | 9.4 | 30.6 | 90.6 | 69.4 | 54 | 94.9 | 96.1 | 46.9 | 0.051 | 0.539 | 0 |
| 0.78 | 48 | 93 | 5 | 41 | 75.4 | 53.9 | 94.9 | 9.4 | 30.6 | 90.6 | 69.4 | 54 | 94.9 | 96.1 | 46.9 | 0.051 | 0.539 | 0 |
| 0.79 | 47 | 93 | 5 | 42 | 74.9 | 52.8 | 94.9 | 9.6 | 31.1 | 90.4 | 68.9 | 53.5 | 94.8 | 96 | 46.3 | 0.051 | 0.528 | 0 |
| 0.8 | 46 | 93 | 5 | 43 | 74.3 | 51.7 | 94.9 | 9.8 | 31.6 | 90.2 | 68.4 | 53 | 94.6 | 95.9 | 45.7 | 0.051 | 0.517 | 0 |
| 0.81 | 45 | 93 | 5 | 44 | 73.8 | 50.6 | 94.9 | 10 | 32.1 | 90 | 67.9 | 52.4 | 94.5 | 95.9 | 45.1 | 0.051 | 0.506 | 0 |
| 0.82 | 45 | 93 | 5 | 44 | 73.8 | 50.6 | 94.9 | 10 | 32.1 | 90 | 67.9 | 52.4 | 94.5 | 95.9 | 45.1 | 0.051 | 0.506 | 0 |
| 0.83 | 44 | 93 | 5 | 45 | 73.3 | 49.4 | 94.9 | 10.2 | 32.6 | 89.8 | 67.4 | 51.8 | 94.4 | 95.8 | 44.6 | 0.051 | 0.494 | 0 |
| 0.84 | 44 | 93 | 5 | 45 | 73.3 | 49.4 | 94.9 | 10.2 | 32.6 | 89.8 | 67.4 | 51.8 | 94.4 | 95.8 | 44.6 | 0.051 | 0.494 | 0 |
| 0.85 | 43 | 94 | 4 | 46 | 73.3 | 48.3 | 95.9 | 8.5 | 32.9 | 91.5 | 67.1 | 56.8 | 94.4 | 96.5 | 44.3 | 0.0408 | 0.483 | 0.004987 |
| 0.86 | 41 | 94 | 4 | 48 | 72.2 | 46.1 | 95.9 | 8.9 | 33.8 | 91.1 | 66.2 | 55.6 | 94.1 | 96.3 | 43.3 | 0.0408 | 6.461 | 0 |
| 0.87 | 40 | 94 | 4 | 49 | 71.7 | 44.9 | 95.9 | 9 1 | 34.3 | 90.9 | 65.7 | 55 | 94 | 96.3 | 42.7 | 0.0408 | 0.449 | 0 |
| 0.88 | 35 | 95 | 3 | 54 | 69.5 | 39.3 | 96.9 | 7.9 | 36.2 | 92.1 | 63.8 | 58.8 | 93.5 | 96.8 | 40.6 | 0.0306 | 0.393 | 0.004299 |
| 0.89 | 33 | 95 | 3 | 56 | 68.4 | 37.1 | 96.9 | 8.3 | 37.1 | 91.7 | 62.9 | 57.4 | 93.3 | 96.6 | 39.8 | 0.0306 | 0.371 | 0 |
| 0.9 | 33 | 95 | 3 | 56 | 68.4 | 37.1 | 96.9 | 8.3 | 37.1 | 91.7 | 62.9 | 57.4 | 93.3 | 96.6 | 39.8 | 0.0306 | 0.371 | 0 |
| 0.91 | 33 | 95 | 3 | 56 | 68.4 | 37.1 | 96.9 | 8.3 | 37.1 | 91.7 | 62.9 | 57.4 | 93.3 | 96.6 | 39.8 | 0.0306 | 0.371 | 0 |
| 0.92 | 33 | 96 | 2 | 56 | 69 | 37.1 | 98 | 5.7 | 36.8 | 94.3 | 63.2 | 66.9 | 93.3 | 97.7 | 40 | 0.0204 | 0.371 | 0.003784 |
| 0.93 | 33 | 96 | 2 | 56 | 69 | 37.1 | 98 | 5.7 | 36.8 | 94.3 | 63.2 | 66.9 | 93.3 | 977 | 40 | 0.0204 | 0.371 | 0 |
| 0.94 | 30 | 96 | 2 | 59 | 67.4 | 33.7 | 98 | 6.3 | 38.1 | 93.8 | 61.9 | 64.7 | 93 | 97.5 | 38.8 | 0.0204 | 0.337 | 0 |
| 0.95 | 27 | 96 | 2 | 62 | 65.8 | 30.3 | 98 | 6.9 | 39.2 | 93.1 | 60.8 | 62.3 | 92.7 | 97.2 | 37.6 | 0.0204 | 0.303 | 0 |
| 0.96 | 25 | 96 | 2 | 64 | 64.7 | 28.1 | 98 | 7.4 | 40 | 92.6 | 60 | 60.5 | 92.5 | 97 | 36.9 | 0.0204 | 0.281 | 0 |
| 0.97 | 23 | 97 | 1 | 66 | 64.2 | 25.8 | 99 | 4.2 | 40.5 | 95.8 | 59.5 | 73.8 | 92.3 | 98.3 | 36.4 | 0.0102 | 0.258 | 0.002752 |
| 0.98 | 21 | 97 | 1 | 68 | 63.1 | 23.6 | 99 | 4.5 | 41.2 | 95.5 | 58.8 | 72 | 92.1 | 98.2 | 35.7 | 0.0102 | 0.236 | 0 |
| 0.99 | 15 | 97 | 1 | 74 | 59.9 | 16.9 | 99 | 6.3 | 43.3 | 93.8 | 56.7 | 64.7 | 91.5 | 97.5 | 33.8 | 0.0102 | 0.169 | 0 |
| 1 | 0 | 98 | 0 | 89 | 52.4 | 0 | 100 | — | 47.6 | — | 52.4 | — | 90 | — | 30 | 0 | 0 | 0.00086 |
| | | | | | | | | | | | | | | | | | AUC | 0.871016 |

TABLE 10C
Analysis of Training plus Testing Set using Six Variable Model; n = 368 stage 1 = 185 stage 0 = 183; Stage 1 (evt) vs. Stage 0 (non-evt).

| | | | | | | | | | | Prevalence | | | | | | | | |
|---|---|---|---|---|---|---|---|---|---|---|---|---|---|---|---|---|---|---|
| | correct | | incorrect | | % | | | | | 50.3% | | 10% | | 70% | | | | |
| prob | evt | n-e | evt | n-e | correct | sensi | spec | f-pos | f-neg | PPV | NPV | PPV | NPV | PPV | NPV | 1-Spec | Sensi | AUC |
| 0 | 185 | 0 | 183 | 0 | 50.3 | 100.0 | 0.0 | 49.7 | — | 50.3 | — | 10.0 | — | 70.0 | — | 1 | 1 | |
| 0.01 | 185 | 0 | 183 | 0 | 50.3 | 100.0 | 0.0 | 49.7 | — | 50.3 | — | 10.0 | — | 70.0 | — | 1 | 1 | 0 |
| 0.02 | 185 | 1 | 182 | 0 | 50.5 | 100.0 | 0.5 | 49.6 | 0.0 | 50.4 | 100.0 | 10.0 | 100.0 | 70.1 | 100.0 | 0.9945 | 1 | 0.005464 |
| 0.03 | 184 | 8 | 175 | 1 | 52.2 | 99.5 | 4.4 | 48.7 | 11.1 | 51.3 | 88.9 | 10.4 | 98.6 | 70.8 | 77.6 | 0.9563 | 0.995 | 0.038148 |
| 0.04 | 183 | 12 | 171 | 2 | 53.0 | 98.9 | 6.6 | 48.3 | 14.3 | 51.7 | 85.7 | 10.5 | 98.2 | 71.2 | 72.2 | 0.9344 | 0.989 | 0.021681 |
| 0.05 | 183 | 19 | 164 | 2 | 54.9 | 98.9 | 10.4 | 47.3 | 9.5 | 52.7 | 90.5 | 10.9 | 98.9 | 72.0 | 80.5 | 0.8962 | 0.989 | 0.037838 |
| 0.06 | 183 | 25 | 158 | 2 | 56.5 | 98.9 | 13.7 | 46.3 | 7.4 | 53.7 | 92.6 | 11.3 | 99.1 | 72.8 | 844 | 0.8634 | 0.989 | 0.032432 |
| 0.07 | 183 | 29 | 154 | 2 | 57.6 | 98.9 | 15.8 | 45.7 | 6.5 | 54.3 | 93.5 | 11.6 | 99.2 | 73.3 | 86.3 | 0.8415 | 0.989 | 0.021622 |
| 0.08 | 183 | 33 | 150 | 2 | 58.7 | 98.9 | 18.0 | 45.0 | 5.7 | 55.0 | 94.3 | 11.8 | 99.3 | 73.8 | 87.7 | 0.8197 | 0.989 | 0.021622 |
| 0.09 | 183 | 36 | 147 | 2 | 59.5 | 98.9 | 19.7 | 44.5 | 5.3 | 55.5 | 94.7 | 12.0 | 99.4 | 74.2 | 88.6 | 0.8033 | 0.989 | 0.016216 |
| 0.1 | 183 | 40 | 143 | 2 | 60.6 | 98.9 | 21.9 | 43.9 | 4.8 | 56.1 | 95.2 | 12.3 | 99.5 | 74.7 | 89.7 | 0.7814 | 0.989 | 0.021622 |
| 0.11 | 183 | 41 | 142 | 2 | 60.9 | 98.9 | 22.4 | 43.7 | 4.7 | 56.3 | 95.3 | 12.4 | 99.5 | 74.8 | 89.9 | 0.776 | 0.989 | 0.005405 |
| 0.12 | 183 | 46 | 137 | 2 | 62.2 | 98.9 | 25.1 | 42.8 | 4.2 | 57.2 | 95.8 | 12.8 | 99.5 | 75.5 | 90.9 | 0.7486 | 0.989 | 0.027027 |
| 0.13 | 182 | 51 | 132 | 3 | 63.3 | 98.4 | 27.9 | 42.0 | 5.6 | 58.0 | 94.4 | 13.2 | 99.4 | 76.1 | 88.0 | 0.7213 | 0.984 | 0.026953 |
| 0.14 | 181 | 54 | 129 | 4 | 63.9 | 97.8 | 29.5 | 41.6 | 6.9 | 58.4 | 93.1 | 13.4 | 99.2 | 76.4 | 85.4 | 0.7049 | 0.978 | 0.016083 |
| 0.15 | 181 | 60 | 123 | 4 | 65.5 | 97.8 | 32.8 | 40.5 | 6.3 | 59.5 | 93.8 | 13.9 | 99.3 | 77.3 | 86.7 | 0.6721 | 0.978 | 0.032078 |
| 0.16 | 181 | 68 | 115 | 4 | 67.7 | 97.8 | 37.2 | 38.9 | 5.6 | 61.1 | 94.4 | 14.7 | 99.4 | 78.4 | 88.0 | 0.6284 | 0.978 | 0.042771 |
| 0.17 | 178 | 72 | 111 | 7 | 67.9 | 96.2 | 39.3 | 38.4 | 8.9 | 61.6 | 91.1 | 15.0 | 98.9 | 78.7 | 81.7 | 0.6066 | 0.962 | 0.021208 |
| 0.18 | 178 | 77 | 106 | 7 | 69.3 | 96.2 | 42.1 | 37.3 | 8.3 | 62.7 | 91.7 | 15.6 | 99.0 | 79.5 | 82.7 | 0.5792 | 0.962 | 0.026289 |
| 0.19 | 178 | 81 | 102 | 7 | 70.4 | 96.2 | 44.3 | 36.4 | 8.0 | 63.6 | 92.0 | 16.1 | 99.1 | 80.1 | 83.4 | 0.5574 | 0.962 | 0.021031 |
| 0.2 | 178 | 86 | 97 | 7 | 71.7 | 96.2 | 47.0 | 35.3 | 7.5 | 64.7 | 92.5 | 16.8 | 99.1 | 80.9 | 84.2 | 0.5301 | 0.962 | 0.026289 |
| 0.21 | 175 | 91 | 92 | 10 | 72.3 | 94.6 | 49.7 | 34.5 | 9.9 | 65.5 | 90.1 | 17.3 | 98.8 | 81.4 | 79.8 | 0.5027 | 0.946 | 0.026067 |
| 0.22 | 175 | 96 | 87 | 10 | 73.6 | 94.6 | 52.5 | 33.2 | 9.4 | 66.8 | 90.6 | 18.1 | 98.9 | 82.3 | 80.6 | 0.4754 | 0.946 | 0.025846 |
| 0.23 | 173 | 100 | 83 | 12 | 74.2 | 93.5 | 54.6 | 32.4 | 10.7 | 67.6 | 89.3 | 18.6 | 98.7 | 82.8 | 78.3 | 0.4536 | 0.935 | 0.020558 |
| 0.24 | 173 | 103 | 80 | 12 | 75.0 | 93.5 | 56.3 | 31.6 | 10.4 | 68.4 | 89.6 | 19.2 | 98.7 | 83.3 | 78.8 | 0.4372 | 0.935 | 0.01533 |
| 0.25 | 173 | 104 | 79 | 12 | 75.3 | 93.5 | 56.8 | 31.3 | 10.3 | 68.7 | 89.7 | 19.4 | 98.7 | 83.5 | 79.0 | 0.4317 | 0.935 | 0.00511 |
| 0.26 | 172 | 108 | 75 | 13 | 76.1 | 93.0 | 59.0 | 30.4 | 10.7 | 69.6 | 89.3 | 20.1 | 98.7 | 84.1 | 78.3 | 0.4098 | 0.93 | 0.020381 |
| 0.27 | 171 | 112 | 71 | 14 | 76.9 | 92.4 | 61.2 | 29.3 | 11.1 | 70.7 | 88.9 | 20.9 | 98.6 | 84.8 | 77.6 | 0.388 | 0.924 | 0.020263 |
| 0.28 | 170 | 114 | 69 | 15 | 77.2 | 91.9 | 62.3 | 28.9 | 11.6 | 71.1 | 88.4 | 21.3 | 98.6 | 85.0 | 76.7 | 0.377 | 0.919 | 0.010072 |
| 0.29 | 167 | 114 | 69 | 18 | 76.4 | 90.3 | 62.3 | 29.2 | 13.6 | 70.8 | 86.4 | 21.0 | 98.3 | 84.8 | 73.3 | 0.377 | 0.903 | 0 |
| 0.3 | 167 | 114 | 69 | 18 | 76.4 | 90.3 | 62.3 | 29.2 | 13.6 | 70.8 | 86.4 | 21.0 | 98.3 | 84.8 | 73.3 | 0.377 | 0.903 | 0 |
| 0.31 | 167 | 115 | 68 | 18 | 76.6 | 90.3 | 62.8 | 28.9 | 13.5 | 71.1 | 86.5 | 21.3 | 98.3 | 85.0 | 73.5 | 0.3716 | 0.903 | 0.004933 |
| 0.32 | 167 | 118 | 65 | 18 | 77.4 | 90.3 | 64.5 | 28.0 | 13.2 | 72.0 | 86.8 | 22.0 | 98.4 | 85.6 | 74.0 | 0.3552 | 0.903 | 0.014798 |
| 0.33 | 167 | 121 | 62 | 18 | 78.3 | 90.3 | 66.1 | 27.1 | 12.9 | 72.9 | 87.1 | 22.8 | 98.4 | 86.1 | 74.4 | 0.3388 | 0.903 | 0.014798 |
| 0.34 | 166 | 124 | 59 | 19 | 78.8 | 89.7 | 67.8 | 26.2 | 13.3 | 73.8 | 86.7 | 23.6 | 98.3 | 86.7 | 73.9 | 0.3224 | 0.897 | 0.014754 |
| 0.35 | 166 | 127 | 56 | 19 | 79.6 | 89.7 | 69.4 | 25.2 | 13.0 | 74.8 | 87.0 | 24.6 | 98.4 | 87.2 | 74.3 | 0.306 | 0.897 | 0.01471 |
| 0.36 | 166 | 128 | 55 | 19 | 79.9 | 89.7 | 69.9 | 24.9 | 12.9 | 75.1 | 87.1 | 24.9 | 98.4 | 87.4 | 74.5 | 0.3005 | 0.897 | 0.004903 |
| 0.37 | 165 | 130 | 53 | 20 | 80.2 | 89.2 | 71.0 | 24.3 | 13.3 | 75.7 | 86.7 | 25.5 | 98.3 | 87.8 | 73.8 | 0.2896 | 0.892 | 0.009777 |
| 0.38 | 164 | 130 | 53 | 21 | 79.9 | 88.6 | 71.0 | 24.4 | 13.9 | 75.6 | 86.1 | 25.4 | 98.3 | 87.7 | 72.8 | 0.2896 | 0.886 | 0 |
| 0.39 | 162 | 133 | 50 | 23 | 80.2 | 87.6 | 72.7 | 23.6 | 14.7 | 76.4 | 85.3 | 26.3 | 98.1 | 88.2 | 71.5 | 0.2732 | 0.876 | 0.014444 |
| 0.4 | 162 | 133 | 50 | 23 | 80.2 | 87.6 | 72.7 | 23.6 | 14.7 | 76.4 | 85.3 | 26.3 | 98.1 | 88.2 | 71.5 | 0.2732 | 0.876 | 0 |
| 0.41 | 161 | 134 | 49 | 24 | 80.2 | 87.0 | 73.2 | 23.3 | 15.2 | 76.7 | 84.8 | 26.5 | 98.1 | 88.4 | 70.8 | 0.2678 | 0.87 | 0.00477 |
| 0.42 | 160 | 134 | 49 | 25 | 79.9 | 86.5 | 73.2 | 23.4 | 15.7 | 76.6 | 84.3 | 26.4 | 98.0 | 88.3 | 69.9 | 0.2678 | 0.865 | 0 |
| 0.43 | 157 | 136 | 47 | 28 | 79.6 | 84.9 | 74.3 | 23.0 | 17.1 | 77.0 | 82.9 | 26.9 | 97.8 | 88.5 | 67.8 | 0.2568 | 0.849 | 0.009363 |
| 0.44 | 157 | 136 | 47 | 28 | 79.6 | 84.9 | 74.3 | 23.0 | 17.1 | 77.0 | 82.9 | 26.9 | 97.8 | 88.5 | 67.8 | 0.2568 | 0.849 | 0 |
| 0.45 | 157 | 136 | 47 | 28 | 79.6 | 84.9 | 74.3 | 23.0 | 17.1 | 77.0 | 82.9 | 26.9 | 97.8 | 88.5 | 67.8 | 0.2568 | 0.849 | 0 |
| 0.46 | 154 | 138 | 45 | 31 | 79.3 | 83.2 | 75.4 | 22.6 | 18.3 | 77.4 | 81.7 | 27.3 | 97.6 | 88.8 | 65.9 | 0.2459 | 0.832 | 0.009186 |
| 0.47 | 152 | 141 | 42 | 33 | 79.6 | 82.2 | 77.0 | 21.6 | 19.0 | 78.4 | 81.0 | 28.5 | 97.5 | 89.3 | 64.9 | 0.2295 | 0.822 | 0.013558 |
| 0.48 | 150 | 143 | 40 | 35 | 79.6 | 81.1 | 78.1 | 21.1 | 19.7 | 78.9 | 80.3 | 29.2 | 97.4 | 89.6 | 63.9 | 0.2186 | 0.811 | 0.00892 |
| 0.49 | 147 | 144 | 39 | 38 | 79.1 | 79.5 | 78.7 | 21.0 | 20.9 | 79.0 | 79.1 | 29.3 | 97.2 | 89.7 | 62.1 | 0.2131 | 0.795 | 0.004386 |
| 0.5 | 147 | 147 | 36 | 38 | 79.9 | 79.5 | 80.3 | 19.7 | 20.5 | 80.3 | 79.5 | 31.0 | 97.2 | 90.4 | 62.6 | 0.1967 | 0.795 | 0.013026 |
| 0.51 | 145 | 149 | 34 | 40 | 79.9 | 78.4 | 81.4 | 19.0 | 21.2 | 81.0 | 78.8 | 31.9 | 97.1 | 90.8 | 61.7 | 0.1858 | 0.784 | 0.008625 |
| 0.52 | 144 | 150 | 33 | 41 | 79.9 | 77.8 | 82.0 | 18.6 | 21.5 | 81.4 | 78.5 | 32.4 | 97.1 | 91.0 | 61.3 | 0.1803 | 0.778 | 0.004268 |
| 0.53 | 143 | 150 | 33 | 42 | 79.6 | 77.3 | 82.0 | 18.8 | 21.9 | 81.3 | 78.1 | 32.3 | 97.0 | 90.9 | 60.7 | 0.1803 | 0.773 | 0 |
| 0.54 | 143 | 151 | 32 | 42 | 79.9 | 77.3 | 82.5 | 18.3 | 21.8 | 81.7 | 78.2 | 32.9 | 97.0 | 91.2 | 60.9 | 0.1749 | 0.773 | 0.004224 |
| 0.55 | 142 | 154 | 29 | 43 | 80.4 | 76.8 | 84.2 | 17.0 | 21.8 | 83.0 | 78.2 | 35.0 | 97.0 | 91.9 | 60.8 | 0.1585 | 0.768 | 0.012627 |
| 0.56 | 140 | 155 | 28 | 45 | 80.2 | 75.7 | 84.7 | 16.7 | 22.5 | 83.3 | 77.5 | 35.5 | 96.9 | 92.0 | 59.9 | 0.153 | 0.757 | 0.004165 |
| 0.57 | 138 | 157 | 26 | 47 | 80.2 | 74.6 | 85.8 | 15.9 | 23.0 | 84.1 | 77.0 | 36.8 | 96.8 | 92.5 | 59.1 | 0.1421 | 0.746 | 0.008211 |
| 0.58 | 136 | 159 | 24 | 49 | 80.2 | 73.5 | 86.9 | 15.0 | 23.6 | 85.0 | 76.4 | 38.4 | 96.7 | 92.9 | 58.4 | 0.1311 | 0.735 | 0.008093 |
| 0.59 | 135 | 159 | 24 | 50 | 79.9 | 73.0 | 86.9 | 15.1 | 23.9 | 84.9 | 76.1 | 38.2 | 96.7 | 92.8 | 57.9 | 0.1311 | 0.73 | 0 |
| 0.6 | 135 | 160 | 23 | 50 | 80.2 | 73.0 | 87.4 | 14.6 | 23.8 | 85.4 | 76.2 | 39.2 | 96.7 | 93.1 | 58.1 | 0.1257 | 0.73 | 0.003988 |
| 0.61 | 130 | 161 | 22 | 55 | 79.1 | 70.3 | 88.0 | 14.5 | 25.5 | 85.5 | 74.5 | 39.4 | 96.4 | 93.2 | 55.9 | 0.1202 | 0.703 | 0.003914 |
| 0.62 | 126 | 161 | 22 | 59 | 78.0 | 68.1 | 88.0 | 14.9 | 26.8 | 85.1 | 73.2 | 38.6 | 96.1 | 93.0 | 54.2 | 0.1202 | 0.681 | 0 |
| 0.63 | 124 | 163 | 20 | 61 | 78.0 | 67.0 | 89.1 | 13.9 | 27.2 | 86.1 | 72.8 | 40.5 | 96.0 | 93.5 | 53.7 | 0.1093 | 0.67 | 0.007384 |
| 0.64 | 124 | 164 | 19 | 61 | 78.3 | 67.0 | 89.6 | 13.3 | 27.1 | 86.7 | 72.9 | 41.8 | 96.1 | 93.8 | 53.8 | 0.1038 | 0.67 | 0.003663 |
| 0.65 | 122 | 165 | 18 | 63 | 78.0 | 65.9 | 90.2 | 12.9 | 27.6 | 87.1 | 72.4 | 42.7 | 96.0 | 94.0 | 53.2 | 0.0984 | 0.659 | 0.003633 |
| 0.66 | 118 | 166 | 17 | 67 | 77.2 | 63.8 | 90.7 | 12.6 | 28.8 | 87.4 | 71.2 | 43.3 | 95.8 | 94.1 | 51.8 | 0.0929 | 0.638 | 0.003545 |
| 0.67 | 116 | 168 | 15 | 69 | 77.2 | 62.7 | 91.8 | 11.5 | 29.1 | 88.5 | 70.9 | 45.9 | 95.7 | 94.7 | 51.3 | 0.082 | 0.627 | 0.006912 |
| 0.68 | 115 | 169 | 14 | 70 | 77.2 | 62.2 | 92.3 | 10.9 | 29.3 | 89.1 | 70.7 | 47.4 | 95.6 | 95.0 | 51.1 | 0.0765 | 0.622 | 0.003412 |
| 0.69 | 113 | 170 | 13 | 72 | 76.9 | 61.1 | 92.9 | 10.3 | 29.8 | 89.7 | 70.2 | 48.9 | 95.6 | 95.3 | 50.6 | 0.071 | 0.611 | 0.003367 |
| 0.7 | 109 | 170 | 13 | 76 | 75.8 | 58.9 | 92.9 | 10.7 | 30.9 | 89.3 | 69.1 | 48.0 | 95.3 | 95.1 | 49.2 | 0.071 | 0.589 | 0 |
| 0.71 | 108 | 171 | 12 | 77 | 75.8 | 58.4 | 93.4 | 10.0 | 31.0 | 90.0 | 69.0 | 49.7 | 95.3 | 95.4 | 49.0 | 0.0656 | 0.584 | 0.003205 |
| 0.72 | 107 | 171 | 12 | 78 | 75.5 | 57.8 | 93.4 | 10.1 | 31.3 | 89.9 | 68.7 | 49.5 | 95.2 | 95.4 | 48.7 | 0.0656 | 0.578 | 0 |
| 0.73 | 106 | 171 | 12 | 79 | 75.3 | 57.3 | 93.4 | 10.2 | 31.6 | 89.8 | 68.4 | 49.3 | 95.2 | 95.3 | 48.4 | 0.0656 | 0.573 | 0 |
| 0.74 | 106 | 172 | 11 | 79 | 75.5 | 57.3 | 94.0 | 9.4 | 31.5 | 90.6 | 68.5 | 51.4 | 95.2 | 95.7 | 48.5 | 0.0601 | 0.573 | 0.003131 |
| 0.75 | 104 | 173 | 10 | 81 | 75.3 | 56.2 | 94.5 | 8.8 | 31.9 | 91.2 | 68.1 | 53.3 | 95.1 | 96.0 | 48.1 | 0.0546 | 0.562 | 0.003101 |
| 0.76 | 104 | 173 | 10 | 81 | 75.3 | 56.2 | 94.5 | 8.8 | 31.9 | 91.2 | 68.1 | 53.3 | 95.1 | 96.0 | 48.1 | 0.0546 | 0.562 | 0 |
| 0.77 | 102 | 174 | 9 | 83 | 75.0 | 55.1 | 95.1 | 8.1 | 32.3 | 91.9 | 67.7 | 55.5 | 95.0 | 96.3 | 47.6 | 0.0492 | 0.551 | 0.003042 |
| 0.78 | 101 | 175 | 8 | 84 | 75.0 | 54.6 | 95.6 | 7.3 | 32.4 | 92.7 | 67.6 | 58.1 | 95.0 | 96.7 | 47.4 | 0.0437 | 0.546 | 0.002998 |
| 0.79 | 99 | 175 | 8 | 86 | 74.5 | 53.5 | 95.6 | 7.5 | 33.0 | 92.5 | 67.0 | 57.6 | 94.9 | 96.6 | 46.9 | 0.0437 | 0.535 | 0 |
| 0.8 | 98 | 175 | 8 | 87 | 74.2 | 53.0 | 95.6 | 7.5 | 33.2 | 92.5 | 66.8 | 57.4 | 94.8 | 96.6 | 46.6 | 0.0437 | 0.53 | 0 |
| 0.81 | 94 | 175 | 8 | 91 | 73.1 | 50.8 | 95.6 | 7.8 | 34.2 | 92.2 | 65.8 | 56.4 | 94.6 | 96.4 | 45.5 | 0.0437 | 0.508 | 0 |
| 0.82 | 94 | 176 | 7 | 91 | 73.4 | 50.8 | 96.2 | 6.9 | 34.1 | 93.1 | 65.9 | 59.6 | 94.6 | 96.9 | 45.6 | 0.0383 | 0.508 | 0.002777 |
| 0.83 | 93 | 176 | 7 | 92 | 73.1 | 50.3 | 96.2 | 7.0 | 34.3 | 93.0 | 65.7 | 59.4 | 94.6 | 96.8 | 45.3 | 0.0383 | 0.503 | 0 |
| 0.84 | 92 | 176 | 7 | 93 | 72.8 | 49.7 | 96.2 | 7.1 | 34.6 | 92.9 | 65.4 | 59.1 | 94.5 | 96.8 | 45.1 | 0.0383 | 0.497 | 0 |
| 0.85 | 91 | 177 | 6 | 94 | 72.8 | 49.2 | 96.7 | 6.2 | 34.7 | 93.8 | 65.3 | 62.5 | 94.5 | 97.2 | 44.9 | 0.0328 | 0.492 | 0.002703 |
| 0.86 | 88 | 177 | 6 | 97 | 72.0 | 47.6 | 96.7 | 6.4 | 35.4 | 93.6 | 64.6 | 61.7 | 94.3 | 97.1 | 44.2 | 0.0328 | 0.476 | 0 |
| 0.87 | 85 | 177 | 6 | 100 | 71.2 | 45.9 | 96.7 | 6.6 | 36.1 | 93.4 | 63.9 | 60.9 | 94.2 | 97.0 | 43.4 | 0.0328 | 0.459 | 0 |
| 0.88 | 76 | 178 | 5 | 109 | 69.0 | 41.1 | 97.3 | 6.2 | 38.0 | 93.8 | 62.0 | 62.6 | 93.7 | 97.2 | 41.4 | 0.0273 | 0.411 | 0.002378 |
| 0.89 | 73 | 178 | 5 | 112 | 68.2 | 39.5 | 97.3 | 6.4 | 38.6 | 93.6 | 61.4 | 61.6 | 93.5 | 97.1 | 40.8 | 0.0273 | 0.395 | 0 |
| 0.9 | 72 | 178 | 5 | 113 | 67.9 | 38.9 | 97.3 | 6.5 | 38.8 | 93.5 | 61.2 | 61.3 | 93.5 | 97.1 | 40.6 | 0.0273 | 0.389 | 0 |
| 0.91 | 72 | 178 | 5 | 113 | 67.9 | 38.9 | 97.3 | 6.5 | 38.8 | 93.5 | 61.2 | 61.3 | 93.5 | 97.1 | 40.6 | 0.0273 | 0.389 | 0 |
| 0.92 | 70 | 179 | 4 | 115 | 67.7 | 37.8 | 97.8 | 5.4 | 39.1 | 94.6 | 60.9 | 65.8 | 93.4 | 97.6 | 40.3 | 0.0219 | 0.378 | 0.002097 |
| 0.93 | 70 | 179 | 4 | 115 | 67.7 | 37.8 | 97.8 | 5.4 | 39.1 | 94.6 | 60.9 | 65.8 | 93.4 | 97.6 | 40.3 | 0.0219 | 0.378 | 0 |
| 0.94 | 64 | 180 | 3 | 121 | 66.3 | 34.6 | 98.4 | 4.5 | 40.2 | 95.5 | 59.8 | 70.1 | 93.1 | 98.0 | 39.2 | 0.0164 | 0.346 | 0.001979 |
| 0.95 | 60 | 180 | 3 | 125 | 65.2 | 32.4 | 98.4 | 4.8 | 41.0 | 95.2 | 59.0 | 68.7 | 92.9 | 97.9 | 38.4 | 0.0164 | 0.324 | 0 |
| 0.96 | 53 | 180 | 3 | 132 | 63.3 | 28.6 | 98.4 | 5.4 | 42.3 | 94.6 | 57.7 | 66.0 | 92.5 | 97.6 | 37.1 | 0.0164 | 0.286 | 0 |
| 0.97 | 48 | 181 | 2 | 137 | 62.2 | 25.9 | 98.9 | 4.0 | 43.1 | 96.0 | 56.9 | 72.5 | 92.3 | 98.2 | 36.4 | 0.0109 | 0.259 | 0.001492 |
| 0.98 | 42 | 181 | 2 | 143 | 60.6 | 22.7 | 98.9 | 4.5 | 44.1 | 95.5 | 55.9 | 69.8 | 92.0 | 98.0 | 35.4 | 0.0109 | 0.227 | 0 |
| 0.99 | 31 | 182 | 1 | 154 | 57.9 | 16.8 | 99.5 | 3.1 | 45.8 | 96.9 | 54.2 | 77.3 | 91.5 | 98.6 | 33.9 | 0.0055 | 0.168 | 0.001078 |
| 1 | 0 | 183 | 0 | 185 | 49.7 | 0.0 | 100.0 | — | 50.3 | — | 49.7 | — | 90.0 | — | 30.0 | 0 | 0 | 0.000458 |
| | | | | | | | | | | | | | | | | | AUC | 0.882204 |

TABLE 10D
Analysis of Training plus Testing Set Excluding Singlets using Six Variable Model; n = 268; Stage 1 (evt) vs. Stage 0 (non-evt).

| | | | | | | | | | | Prevalence | | | | | | | | |
|---|---|---|---|---|---|---|---|---|---|---|---|---|---|---|---|---|---|---|
| | correct | | incorrect | | % | | | | | 55.0% | | 10% | | 70% | | | | |
| prob | evt | n-e | evt | n-e | correct | sensi | spec | f-pos | f-neg | PPV | NPV | PPV | NPV | PPV | NPV | 1-Spec | Sensi | AUC |
| 0 | 147 | 0 | 121 | 0 | 54.9 | 100 | 0 | 45.1 | — | 54.9 | — | 10 | — | 70 | — | 1 | 1 | |
| 0.01 | 147 | 0 | 121 | 0 | 54.9 | 100 | 0 | 45.1 | — | 54.9 | — | 10 | — | 70 | — | 1 | 1 | 0 |
| 0.02 | 147 | 1 | 120 | 0 | 55.2 | 100 | 0.8 | 44.9 | 0 | 55.1 | 100 | 10.1 | 100 | 70.2 | 100 | 0.992 | 1 | 0.008264 |
| 0.03 | 146 | 6 | 115 | 1 | 56.7 | 99.3 | 5 | 44.1 | 14.3 | 55.9 | 85.7 | 10.4 | 98.5 | 70.9 | 75.8 | 0.95 | 0.993 | 0.041182 |
| 0.04 | 145 | 8 | 113 | 2 | 57.1 | 98.6 | 6.6 | 43.8 | 20 | 56.2 | 80 | 10.5 | 97.8 | 71.1 | 67.6 | 0.934 | 0.986 | 0.01636 |
| 0.05 | 145 | 13 | 108 | 2 | 59 | 98.6 | 10.7 | 42.7 | 13.3 | 57.3 | 86.7 | 10.9 | 98.6 | 72.1 | 77.2 | 0.893 | 0.986 | 0.04076 |
| 0.06 | 145 | 18 | 103 | 2 | 60.8 | 98.6 | 14.9 | 41.5 | 10 | 58.5 | 90 | 11.4 | 99 | 73 | 82.4 | 0.851 | 0.986 | 0.04076 |
| 0.07 | 145 | 18 | 103 | 2 | 60.8 | 98.6 | 14.9 | 41.5 | 10 | 58.5 | 90 | 11.4 | 99 | 73 | 82.4 | 0.851 | 0.986 | 0 |
| 0.08 | 145 | 22 | 99 | 2 | 62.3 | 98.6 | 18.2 | 40.6 | 8.3 | 59.4 | 91.7 | 11.8 | 99.2 | 73.8 | 85.1 | 0.818 | 0.986 | 0.032608 |
| 0.09 | 145 | 25 | 96 | 2 | 63.4 | 98.6 | 20.7 | 39.8 | 7.4 | 60.2 | 92.6 | 12.1 | 99.3 | 74.4 | 86.7 | 0.793 | 0.986 | 0.024456 |
| 0.1 | 145 | 26 | 95 | 2 | 63.8 | 98.6 | 21.5 | 39.6 | 7.1 | 60.4 | 92.9 | 12.2 | 99.3 | 74.6 | 87.1 | 0.785 | 0.986 | 0.008152 |
| 0.11 | 145 | 27 | 94 | 2 | 64.2 | 98.6 | 22.3 | 39.3 | 6.9 | 60.7 | 93.1 | 12.4 | 99.3 | 74.8 | 87.5 | 0.777 | 0.986 | 0.008152 |
| 0.12 | 145 | 32 | 89 | 2 | 66 | 98.6 | 26.4 | 38 | 5.9 | 62 | 94.1 | 13 | 99.4 | 75.8 | 89.3 | 0.736 | 0.986 | 0.04076 |
| 0.13 | 145 | 35 | 86 | 2 | 67.2 | 98.6 | 28.9 | 37.2 | 5.4 | 62.8 | 94.6 | 13.4 | 99.5 | 76.4 | 90.1 | 0.711 | 0.986 | 0.024456 |
| 0.14 | 145 | 37 | 84 | 2 | 67.9 | 98.6 | 30.6 | 36.7 | 5.1 | 63.3 | 94.9 | 13.6 | 99.5 | 76.8 | 90.6 | 0.694 | 0.986 | 0.016304 |
| 0.15 | 145 | 41 | 80 | 2 | 69.4 | 98.6 | 33.9 | 35.6 | 4.7 | 64.4 | 95.3 | 14.2 | 99.6 | 77.7 | 91.4 | 0.661 | 0.986 | 0.032608 |
| 0.16 | 145 | 45 | 76 | 2 | 70.9 | 98.6 | 37.2 | 34.4 | 4.3 | 65.6 | 95.7 | 14.9 | 99.6 | 78.6 | 92.1 | 0.628 | 0.986 | 0.032608 |
| 0.17 | 143 | 48 | 73 | 4 | 71.3 | 97.3 | 39.7 | 33.8 | 7.7 | 66.2 | 92.3 | 15.2 | 99.2 | 79 | 86.2 | 0.603 | 0.973 | 0.024287 |
| 0.18 | 143 | 51 | 70 | 4 | 72.4 | 97.3 | 42.1 | 32.9 | 7.3 | 67.1 | 92.7 | 15.7 | 99.3 | 79.7 | 86.9 | 0.579 | 0.973 | 0.024119 |
| 0.19 | 143 | 55 | 66 | 4 | 73.9 | 97.3 | 45.5 | 31.6 | 6.8 | 68.4 | 93.2 | 16.5 | 99.3 | 80.6 | 87.7 | 0.545 | 0.973 | 0.032158 |
| 0.2 | 143 | 57 | 64 | 4 | 74.6 | 97.3 | 47.1 | 30.9 | 6.6 | 69.1 | 93.4 | 17 | 99.4 | 81.1 | 88.1 | 0.529 | 0.973 | 0.016079 |
| 0.21 | 142 | 59 | 62 | 5 | 75 | 96.6 | 48.8 | 30.4 | 7.8 | 69.6 | 92.2 | 17.3 | 99.2 | 81.5 | 86 | 0.512 | 0.966 | 0.016023 |
| 0.22 | 142 | 62 | 59 | 5 | 76.1 | 96.6 | 51.2 | 29.4 | 7.5 | 70.6 | 92.5 | 18 | 99.3 | 82.2 | 86.6 | 0.488 | 0.966 | 0.02395 |
| 0.23 | 141 | 66 | 55 | 6 | 77.2 | 95.9 | 54.5 | 28.1 | 8.3 | 71.9 | 91.7 | 19 | 99.2 | 83.1 | 85.1 | 0.455 | 0.959 | 0.031821 |
| 0.24 | 141 | 68 | 53 | 6 | 78 | 95.9 | 56.2 | 27.3 | 8.1 | 72.7 | 91.9 | 19.6 | 99.2 | 83.6 | 85.5 | 0.438 | 0.959 | 0.015854 |
| 0.25 | 141 | 69 | 52 | 6 | 78.4 | 95.9 | 57 | 26.9 | 8 | 73.1 | 92 | 19.9 | 99.2 | 83.9 | 85.7 | 0.43 | 0.959 | 0.007927 |
| 0.26 | 140 | 71 | 50 | 7 | 78.7 | 95.2 | 58.7 | 26.3 | 9 | 73.7 | 91 | 20.4 | 99.1 | 84.3 | 84.1 | 0.413 | 0.952 | 0.015798 |
| 0.27 | 140 | 74 | 47 | 7 | 79.9 | 95.2 | 61.2 | 25.1 | 8.6 | 74.9 | 91.4 | 21.4 | 99.1 | 85.1 | 84.6 | 0.388 | 0.952 | 0.023613 |
| 0.28 | 139 | 75 | 46 | 8 | 79.9 | 94.6 | 62 | 24.9 | 9.6 | 75.1 | 90.4 | 21.7 | 99 | 85.3 | 83 | 0.38 | 0.946 | 0.007843 |
| 0.29 | 137 | 75 | 46 | 10 | 79.1 | 93.2 | 62 | 25.1 | 11.8 | 74.9 | 88.2 | 21.4 | 98.8 | 85.1 | 79.6 | 0.38 | 0.932 | 0 |
| 0.3 | 137 | 75 | 46 | 10 | 79.1 | 93.2 | 62 | 25.1 | 11.8 | 74.9 | 88.2 | 21.4 | 98.8 | 85.1 | 79.6 | 0.38 | 0.932 | 0 |
| 0.31 | 137 | 76 | 45 | 10 | 79.5 | 93.2 | 62.8 | 24.7 | 11.6 | 75.3 | 88.4 | 21.8 | 98.8 | 85.4 | 79.8 | 0.372 | 0.932 | 0.007702 |
| 0.32 | 137 | 77 | 44 | 10 | 79.9 | 93.2 | 63.6 | 24.3 | 11.5 | 75.7 | 88.5 | 22.2 | 98.8 | 85.7 | 80 | 0.364 | 0.932 | 0.007702 |
| 0.33 | 137 | 78 | 43 | 10 | 80.2 | 93.2 | 64.5 | 23.9 | 11.4 | 76.1 | 88.6 | 22.6 | 98.8 | 86 | 80.2 | 0.355 | 0.932 | 0.007702 |
| 0.34 | 137 | 80 | 41 | 10 | 81 | 93.2 | 66.1 | 23 | 11.1 | 77 | 88.9 | 23.4 | 98.9 | 86.5 | 80.6 | 0.339 | 0.932 | 0.015405 |
| 0.35 | 137 | 82 | 39 | 10 | 81.7 | 93.2 | 67.8 | 22.2 | 10.9 | 77.8 | 89.1 | 24.3 | 98.9 | 87.1 | 81 | 0.322 | 0.932 | 0.015405 |
| 0.36 | 137 | 83 | 38 | 10 | 82.1 | 93.2 | 68.6 | 21.7 | 10.8 | 78.3 | 89.2 | 24.8 | 98.9 | 87.4 | 81.2 | 0.314 | 0.932 | 0.007702 |
| 0.37 | 136 | 85 | 36 | 11 | 82.5 | 92.5 | 70.2 | 20.9 | 11.5 | 79.1 | 88.5 | 25.7 | 98.8 | 87.9 | 80.1 | 0.298 | 0.925 | 0.015348 |
| 0.38 | 136 | 85 | 36 | 11 | 82.5 | 92.5 | 70.2 | 20.9 | 11.5 | 79.1 | 88.5 | 25.7 | 98.8 | 87.9 | 80.1 | 0.298 | 0.925 | 0 |
| 0.39 | 135 | 88 | 33 | 12 | 83.2 | 91.8 | 72.7 | 19.6 | 12 | 80.4 | 88 | 27.2 | 98.8 | 88.7 | 79.2 | 0.273 | 0.918 | 0.022854 |
| 0.4 | 135 | 88 | 33 | 12 | 83.2 | 91.8 | 72.7 | 19.6 | 12 | 80.4 | 88 | 27.2 | 98.8 | 88.7 | 79.2 | 0.273 | 0.918 | 0 |
| 0.41 | 135 | 88 | 33 | 12 | 83.2 | 91.8 | 72.7 | 19.6 | 12 | 80.4 | 88 | 27.2 | 98.8 | 88.7 | 79.2 | 0.273 | 0.918 | 0 |
| 0.42 | 134 | 88 | 33 | 13 | 82.8 | 91.2 | 72.7 | 19.8 | 12.9 | 80.2 | 87.1 | 27.1 | 98.7 | 88.6 | 77.9 | 0.273 | 0.912 | 0 |
| 0.43 | 133 | 90 | 31 | 14 | 83.2 | 90.5 | 74.4 | 18.9 | 13.5 | 81.1 | 86.5 | 28.2 | 98.6 | 89.2 | 77 | 0.256 | 0.905 | 0.015011 |
| 0.44 | 133 | 90 | 31 | 14 | 83.2 | 90.5 | 74.4 | 18.9 | 13.5 | 81.1 | 86.5 | 28.2 | 98.6 | 89.2 | 77 | 0.256 | 0.905 | 0 |
| 0.45 | 133 | 90 | 31 | 14 | 83.2 | 90.5 | 74.4 | 18.9 | 13.5 | 81.1 | 86.5 | 28.2 | 98.6 | 89.2 | 77 | 0.256 | 0.905 | 0 |
| 0.46 | 131 | 92 | 29 | 16 | 83.2 | 89.1 | 76 | 18.1 | 14.8 | 81.9 | 85.2 | 29.2 | 98.4 | 89.7 | 75 | 0.24 | 0.891 | 0.014842 |
| 0.47 | 130 | 94 | 27 | 17 | 83.6 | 88.4 | 77.7 | 17.2 | 15.3 | 82.8 | 84.7 | 30.6 | 98.4 | 90.2 | 74.2 | 0.223 | 0.884 | 0.014674 |
| 0.48 | 128 | 94 | 27 | 19 | 82.8 | 87.1 | 77.7 | 17.4 | 16.8 | 82.6 | 83.2 | 30.2 | 98.2 | 90.1 | 72 | 0.223 | 0.871 | 0 |
| 0.49 | 126 | 95 | 26 | 21 | 82.5 | 85.7 | 78.5 | 17.1 | 18.1 | 82.9 | 81.9 | 30.7 | 98 | 90.3 | 70.2 | 0.215 | 0.857 | 0.00714 |
| 0.5 | 126 | 98 | 23 | 21 | 83.6 | 85.7 | 81 | 15.4 | 17.6 | 84.6 | 82.4 | 33.4 | 98.1 | 91.3 | 70.8 | 0.19 | 0.857 | 0.021251 |
| 0.51 | 124 | 99 | 22 | 23 | 83.2 | 84.4 | 81.8 | 15.1 | 18.9 | 84.9 | 81.1 | 34 | 97.9 | 91.5 | 69.1 | 0.182 | 0.844 | 0.007028 |
| 0.52 | 123 | 100 | 21 | 24 | 83.2 | 83.7 | 82.6 | 14.6 | 19.4 | 85.4 | 80.6 | 34.9 | 97.9 | 91.8 | 68.4 | 0.174 | 0.837 | 0.006943 |
| 0.53 | 123 | 100 | 21 | 24 | 83.2 | 83.7 | 82.6 | 14.6 | 19.4 | 85.4 | 80.6 | 34.9 | 97.9 | 91.8 | 68.4 | 0.174 | 0.837 | 0 |
| 0.54 | 123 | 101 | 20 | 24 | 83.6 | 83.7 | 83.5 | 14 | 19.2 | 86 | 80.8 | 36 | 97.9 | 92.2 | 68.7 | 0.165 | 0.837 | 0.006915 |
| 0.55 | 122 | 102 | 19 | 25 | 83.6 | 83 | 84.3 | 13.5 | 19.7 | 86.5 | 80.3 | 37 | 97.8 | 92.5 | 68 | 0.157 | 0.83 | 0.006887 |
| 0.56 | 120 | 103 | 18 | 27 | 83.2 | 81.6 | 85.1 | 13 | 20.8 | 87 | 79.2 | 37.9 | 97.7 | 92.8 | 66.5 | 0.149 | 0.816 | 0.006803 |
| 0.57 | 118 | 104 | 17 | 29 | 82.8 | 80.3 | 86 | 12.6 | 21.8 | 87.4 | 78.2 | 38.8 | 97.5 | 93 | 65.1 | 0.14 | 0.803 | 0.00669 |
| 0.58 | 118 | 105 | 16 | 29 | 83.2 | 80.3 | 86.8 | 11.9 | 21.6 | 88.1 | 78.4 | 40.3 | 97.5 | 93.4 | 65.3 | 0.132 | 0.803 | 0.006634 |
| 0.59 | 118 | 105 | 16 | 29 | 83.2 | 80.3 | 86.8 | 11.9 | 21.6 | 88.1 | 78.4 | 40.3 | 97.5 | 93.4 | 65.3 | 0.132 | 0.803 | 0 |
| 0.6 | 118 | 106 | 15 | 29 | 83.6 | 80.3 | 87.6 | 11.3 | 21.5 | 88.7 | 78.5 | 41.8 | 97.6 | 93.8 | 65.6 | 0.124 | 0.803 | 0.006634 |
| 0.61 | 115 | 106 | 15 | 32 | 82.5 | 78.2 | 87.6 | 11.5 | 23.2 | 88.5 | 76.8 | 41.2 | 97.3 | 93.6 | 63.3 | 0.124 | 0.782 | 0 |
| 0.62 | 112 | 106 | 15 | 35 | 81.3 | 76.2 | 87.6 | 11.8 | 24.8 | 88.2 | 75.2 | 40.6 | 97.1 | 93.5 | 61.2 | 0.124 | 0.762 | 0 |
| 0.63 | 111 | 106 | 15 | 36 | 81 | 75.5 | 87.6 | 11.9 | 25.4 | 88.1 | 74.6 | 40.4 | 97 | 93.4 | 60.5 | 0.124 | 0.755 | 0 |
| 0.64 | 111 | 107 | 14 | 36 | 81.3 | 75.5 | 88.4 | 11.2 | 25.2 | 88.8 | 74.8 | 42 | 97 | 93.8 | 60.7 | 0.116 | 0.755 | 0.006241 |
| 0.65 | 109 | 107 | 14 | 38 | 80.6 | 74.1 | 88.4 | 11.4 | 26.2 | 88.6 | 73.8 | 41.6 | 96.9 | 93.7 | 59.4 | 0.116 | 0.741 | 0 |
| 0.66 | 106 | 108 | 13 | 41 | 79.9 | 72.1 | 89.3 | 10.9 | 27.5 | 89.1 | 72.5 | 42.7 | 96.6 | 94 | 57.8 | 0.107 | 0.721 | 0.006044 |
| 0.67 | 104 | 109 | 12 | 43 | 79.5 | 70.7 | 90.1 | 10.3 | 28.3 | 89.7 | 71.7 | 44.2 | 96.5 | 94.3 | 56.9 | 0.099 | 0.707 | 0.005903 |
| 0.68 | 103 | 110 | 11 | 44 | 79.5 | 70.1 | 90.9 | 9.6 | 28.6 | 90.4 | 71.4 | 46.1 | 96.5 | 94.7 | 56.6 | 0.091 | 0.701 | 0.005819 |
| 0.69 | 102 | 110 | 11 | 45 | 79.1 | 69.4 | 90.9 | 9.7 | 29 | 90.3 | 71 | 45.9 | 96.4 | 94.7 | 56 | 0.091 | 0.694 | 0 |
| 0.7 | 98 | 110 | 11 | 49 | 77.6 | 66.7 | 90.9 | 10.1 | 30.8 | 89.9 | 69.2 | 44.9 | 96.1 | 94.5 | 53.9 | 0.091 | 0.667 | 0 |
| 0.71 | 97 | 111 | 10 | 50 | 77.6 | 66 | 91.7 | 9.3 | 31.1 | 90.7 | 68.9 | 47 | 96 | 94.9 | 53.6 | 0.083 | 0.66 | 0.005482 |
| 0.72 | 96 | 111 | 10 | 51 | 77.2 | 65.3 | 91.7 | 9.4 | 31.5 | 90.6 | 68.5 | 46.8 | 96 | 94.9 | 53.1 | 0.083 | 0.653 | 0 |
| 0.73 | 95 | 111 | 10 | 52 | 76.9 | 64.6 | 91.7 | 9.5 | 31.9 | 90.5 | 68.1 | 46.5 | 95.9 | 94.8 | 52.6 | 0.083 | 0.646 | 0 |
| 0.74 | 95 | 111 | 10 | 52 | 76.9 | 64.6 | 91.7 | 9.5 | 31.9 | 90.5 | 68.1 | 46.5 | 95.9 | 94.8 | 52.6 | 0.083 | 0.646 | 0 |
| 0.75 | 94 | 112 | 9 | 53 | 76.9 | 63.9 | 92.6 | 8.7 | 32.1 | 91.3 | 67.9 | 48.9 | 95.9 | 95.3 | 52.4 | 0.074 | 0.639 | 0.005313 |
| 0.76 | 94 | 112 | 9 | 53 | 76.9 | 63.9 | 92.6 | 8.7 | 32.1 | 91.3 | 67.9 | 48.9 | 95.9 | 95.3 | 52.4 | 0.074 | 0.639 | 0 |
| 0.77 | 92 | 113 | 8 | 55 | 76.5 | 62.6 | 93.4 | 8 | 32.7 | 92 | 67.3 | 51.3 | 95.7 | 95.7 | 51.7 | 0.066 | 0.626 | 0.005229 |
| 0.78 | 91 | 114 | 7 | 56 | 76.5 | 61.9 | 94.2 | 7.1 | 32.9 | 92.9 | 67.1 | 54.3 | 95.7 | 96.1 | 51.5 | 0.058 | 0.619 | 0.005144 |
| 0.79 | 89 | 114 | 7 | 58 | 75.7 | 60.5 | 94.2 | 7.3 | 33.7 | 92.7 | 66.3 | 53.8 | 95.6 | 96.1 | 50.6 | 0.058 | 0.605 | 0 |
| 0.8 | 88 | 114 | 7 | 59 | 75.4 | 59.9 | 94.2 | 7.4 | 34.1 | 92.6 | 65.9 | 53.5 | 95.5 | 96 | 50.2 | 0.058 | 0.599 | 0 |
| 0.81 | 86 | 114 | 7 | 61 | 74.6 | 58.5 | 94.2 | 7.5 | 34.9 | 92.5 | 65.1 | 52.9 | 95.3 | 95.9 | 49.3 | 0.058 | 0.585 | 0 |
| 0.82 | 86 | 114 | 7 | 61 | 74.6 | 58.5 | 94.2 | 7.5 | 34.9 | 92.5 | 65.1 | 52.9 | 95.3 | 95.9 | 49.3 | 0.058 | 0.585 | 0 |
| 0.83 | 85 | 114 | 7 | 62 | 74.3 | 57.8 | 94.2 | 7.6 | 35.2 | 92.4 | 64.8 | 52.6 | 95.3 | 95.9 | 48.9 | 0.058 | 0.578 | 0 |
| 0.84 | 85 | 114 | 7 | 62 | 74.3 | 57.8 | 94.2 | 7.6 | 35.2 | 92.4 | 64.8 | 52.6 | 95.3 | 95.9 | 48.9 | 0.058 | 0.578 | 0 |
| 0.85 | 85 | 115 | 6 | 62 | 74.6 | 57.8 | 95 | 6.6 | 35 | 93.4 | 65 | 56.4 | 95.3 | 96.5 | 49.1 | 0.05 | 0.578 | 0.004779 |
| 0.86 | 82 | 115 | 6 | 65 | 73.5 | 55.8 | 95 | 6.8 | 36.1 | 93.2 | 63.9 | 55.6 | 95.1 | 96.3 | 47.9 | 0.05 | 0.558 | 0 |
| 0.87 | 81 | 115 | 6 | 66 | 73.1 | 55.1 | 95 | 6.9 | 36.5 | 93.1 | 63.5 | 55.3 | 95 | 96.3 | 47.6 | 0.05 | 0.551 | 0 |
| 0.88 | 72 | 116 | 5 | 75 | 70.1 | 49 | 95.9 | 6.5 | 39.3 | 93.5 | 60.7 | 56.8 | 94.4 | 96.5 | 44.6 | 0.041 | 0.49 | 0.004301 |
| 0.89 | 70 | 116 | 5 | 77 | 69.4 | 47.6 | 95.9 | 6.7 | 39.9 | 93.3 | 60.1 | 56.1 | 94.3 | 96.4 | 44 | 0.041 | 0.476 | 0 |
| 0.9 | 69 | 116 | 5 | 78 | 69 | 46.9 | 95.9 | 6.8 | 40.2 | 93.2 | 59.8 | 55.8 | 94.2 | 96.4 | 43.6 | 0.041 | 0.469 | 0 |
| 0.91 | 69 | 116 | 5 | 78 | 69 | 46.9 | 95.9 | 6.8 | 40.2 | 93.2 | 59.8 | 55.8 | 94.2 | 96.4 | 43.6 | 0.041 | 0.469 | 0 |
| 0.92 | 67 | 117 | 4 | 80 | 68.7 | 45.6 | 96.7 | 5.6 | 40.6 | 94.4 | 59.4 | 60.5 | 94.1 | 97 | 43.2 | 0.033 | 0.456 | 0.003823 |
| 0.93 | 67 | 117 | 4 | 80 | 68.7 | 45.6 | 96.7 | 5.6 | 40.6 | 94.4 | 59.4 | 60.5 | 94.1 | 97 | 43.2 | 0.033 | 0.456 | 0 |
| 0.94 | 61 | 118 | 3 | 86 | 66.8 | 41.5 | 97.5 | 4.7 | 42.2 | 95.3 | 57.8 | 65 | 93.8 | 97.5 | 41.7 | 0.025 | 0.415 | 0.003598 |
| 0.95 | 58 | 118 | 3 | 89 | 65.7 | 39.5 | 97.5 | 4.9 | 43 | 95.1 | 57 | 63.9 | 93.5 | 97.4 | 40.8 | 0.025 | 0.395 | 0 |
| 0.96 | 53 | 118 | 3 | 94 | 63.8 | 36.1 | 97.5 | 5.4 | 44.3 | 94.6 | 55.7 | 61.8 | 93.2 | 97.1 | 39.5 | 0.025 | 0.361 | 0 |
| 0.97 | 48 | 119 | 2 | 99 | 62.3 | 32.7 | 98.3 | 4 | 45.4 | 96 | 54.6 | 68.7 | 92.9 | 97.9 | 38.5 | 0.017 | 0.327 | 0.002839 |
| 0.98 | 42 | 119 | 2 | 105 | 60.1 | 28.6 | 98.3 | 4.5 | 46.9 | 95.5 | 53.1 | 65.8 | 92.5 | 97.6 | 37.1 | 0.017 | 0.286 | 0 |
| 0.99 | 31 | 120 | 1 | 116 | 56.3 | 21.1 | 99.2 | 3.1 | 49.2 | 96.9 | 50.8 | 73.9 | 91.9 | 98.3 | 35 | 0.008 | 0.211 | 0.002052 |
| 1 | 0 | 121 | 0 | 147 | 45.1 | 0 | 100 | — | 54.9 | — | 45.1 | — | 90 | — | 30 | 0 | 0 | 0.000871 |
| | | | | | | | | | | | | | | | | | AUC | 0.901614 |

We claim:

1. A nonsurgical method of predicting the presence of liver fibrosis in an individual, the method comprising;
(a) determining a single diagnostic score using the levels of three or more markers in a sample, wherein at least three of the markers are selected from the group consisting of α2-macroglobulin (α2MG), hyaluronic acid (HA), matrix metalloproteinase-2 (MMP-2), and Activin A, with the proviso that when the three or more markers do not include Activin A, then the plasma marker TIMP metallopeptidase inhibitor 1 (TIMP-1) is not used in the determination; and
(b) comparing the diagnostic score for the individual to a reference score to determine the presence of liver fibrosis.

2. The method of claim 1, wherein deriving a single diagnostic score further comprises using one or more of the individual's age and the individual's sex.

3. The method of claim 1, wherein the markers used to derive the diagnostic score in step (a) comprise α2-macroglobulin (α2MG), hyaluronic acid (HA), and Activin A.

4. The method of claim 3, wherein the markers used to derive the diagnostic score in step (a) further comprise matrix metalloproteinase-2 (MMP-2)

5. The method of claim 1, wherein the sample is selected from the group consisting of blood, scrum, plasma, urine, saliva and liver tissue.

6. The method of claim 1, wherein deriving a single diagnostic score comprises calculating a diagnostic score with a mathematical algorithm.

7. The method of claim 6, wherein the mathematical algorithm comprises a Six Variable Model according to equation (1) for calculating an intermediate value, y:
y=exp(X₁−(C₁*age)+(C₂*sex)+(C₃*α2MG)+(C₄*HA)+(C₅*MMP-2)+(C₆*Activin A)) (1)
wherein,
−6.88236≦X₁≦−4.58824,
0.05152≦C₁≦0.07728,
0.45344≦C₂≦0.68016,
0.00896≦C₃≦0.01344,
0.00608≦C₄≦0.00912,
0.00912≦C₅≦0.01368,
0.00216≦C₆≦0.00324,
age is in years,
male sex=1, female sex=0,
α2MG is in mg/dL,
HA is in ng/mL,
MMP-2 is in ng/mL, and
Activin A is in pg/mL.

8. The method of claim 7, wherein
X₁=−5.7353,
C₁=0.0644,
C₂=0.5668,
C₃=0.0112,
C₄=0.00760,
C₅=0.0114, and
C₆=0.00270.

9. The method of claim 6, wherein the mathematical algorithm comprises a Five Variable Model according to equation (3) for calculating an intermediate value, y:
y=exp(X₂−(C₇*age)+(C₈*sex)+(C₉*α2MG)+(C₁₀*HA)+(C₁₁*Activin A)) (3)
wherein,
−4.74948≦X₂≦−3.16632,
0.04672≦C₇≦0.07008,
0.38608≦C₈≦0.57912,
0.00880≦C₉≦0.01344,
0.00672≦C₁₀≦0.01008,
0.00912≦C₁₁≦0.01368,
age is in years,
male sex=1, female sex=0,
α2MG is in mg/dL,
HA is in ng/mL, and
Activin A is in pg/mL.

10. The method of claim 9, wherein
X₂=−3.9579,
C₇=0.0584,
C₈=0.4826,
C₉=0.0112,
C₁₀=0.0084, and
C₁₁=0.0036.

11. The method of claim 7, wherein the mathematical algorithm further comprises calculating a diagnostic score, H, from an intermediate value, y, according to equation (2):
H=y/(1+y) (2)
wherein a value of said diagnostic score, H, above a reference score is predictive of significant liver fibrosis.

12. The method of claim 11, wherein said reference score is greater than or equal to 0.425 but less than or equal to 0.575.

13. The method of claim 9, wherein the mathematical algorithm further comprises calculating a diagnostic score, H, from an intermediate value, y, according to equation (2):
H=y/(1+y) (2)
wherein a value of said diagnostic score, H, above a reference score is predictive of significant liver fibrosis.

14. The method of claim 13, wherein said reference score is greater than or equal to 0.425 but less than or equal to 0.575.

15. A nonsurgical method of predicting the presence of liver fibrosis in an individual, the method comprising
a) measuring the levels of three or more of α₂-macroglobulin, hyaluronic acid, matrix metalloproteinase-2, and Activin A;
b) using the levels measured in a) to derive an intermediate value, y, using a mathematical algorithm;
c) calculating a single diagnostic score, H, for the individual from an intermediate value, y, using a mathematical algorithm, wherein the mathematical algorithm comprises equation (2):
H=y/(1+y) (2)
c) comparing the diagnostic score, H, to a cut-off value that is predictive of significant liver fibrosis.

16. The method of claim 15, wherein the levels of α₂-macroglobulin (α2MG), hyaluronic acid (HA), matrix metalloproteinase-2 (MMP-2), and Activin A are used to calculate the intermediate value, y, according to equation (1):
y=exp(X₁−(C₁*age)+(C₂*sex)+(C₃*α2MG)+(C₄*HA)+(C₅*MMP-2)+(C₆*Activin A)) (1)
wherein,
−6.88236≦X₁≦−4.58824,
0.05152≦C₁≦0.07728,
0.45344≦C₂≦0.68016,
0.00896≦C₃≦0.01344,
0.00608≦C₄≦0.00912,
0.00912≦C₅≦0.01368,
0.00216≦C₆≦0.00324,
age is in years,
male sex=1, female sex=0,
α2MG is in mg/dL,
HA is in ng/mL,
MMP-2 is in ng/mL, and
Activin A is in pg/mL.

17. The method of claim 16, wherein
X₁=−5.7353,
C₁=0.0644,
C₂=0.5668,
C₃=0.0112,
C₄=0.00760,
C₅=0.0114, and
C₆=0.00270.

18. The method of claim 15, wherein the levels of α₂-macroglobulin (α2MG), hyaluronic acid (HA), and Activin A are used to calculate the intermediate value, y, according to equation (3):
y=exp(X₂−(C₇*age)+(C₈*sex)+(C₉*α2MG)+(C₁₀*HA)+(C₁₁*Activin A)) (3)
wherein,
−4.74948≦X₂≦−3.16632,
0.04672≦C₇≦0.07008,
0.38608≦C₈≦0.57912,
0.00880≦C₉≦0.01344,
0.00672≦C₁₀≦0.01008,
0.00912≦C₁₁≦0.01368,
age is in years,
male sex=1, female sex=0,
α2MG is in mg/dL,
HA is in ng/mL, and
Activin A is in pg/mL.

19. The method of claim 18, wherein
X₂=−3.9579,
C₇=0.0584,
C₈=0.4826,
C₉=0.0112,
C₁₀=0.0084, and
C₁₁=0.0036.

20. The method of claim 15, wherein the reference score is about 0.5, and wherein a diagnostic score, H, of greater than or equal to about 0.5 is indicative of significant fibrosis or a diagnostic score, H, of less than about 0.5 is indicative of the absence of significant fibrosis.

21. The method of claim 15, wherein the sample is selected from the group consisting of blood, serum, plasma, urine, saliva and liver tissue.

22. A method of monitoring liver fibrosis in an individual, comprising the steps of:
(a) determining a first diagnostic score for the individual using the levels of three or more markers in a first sample, wherein the three or more markers are selected from the group consisting of α₂-macroglobulin (α2MG), hyaluronic acid (HA), matrix metalloproteinase-2 (MMP-2), and Activin A, with the proviso that when the three or more markers do not comprise Activin A, TIMP-1 is not used in the determination;
(b) comparing the first diagnostic score for the individual to a reference score to determine the progression of liver fibrosis as indicated by the first sample;
(c) repeating steps (a)-(b) at some later point in time to determine the progression of liver fibrosis as indicated by a second sample; and
(d) monitoring liver fibrosis in an individual by comparing the progression of liver fibrosis indicated by the first sample to the progression of liver fibrosis indicated by the second sample.

23. A device configured to nonsurgically predict the presence of liver fibrosis in an individual, the device comprising:
an input interface configured to receive data, wherein the data comprise age, sex, and levels of α₂-macroglobulin (α2MG), hyaluronic acid (HA), matrix metalloproteinase-2 (MMP-2), and Activin A in a sample from the individual,
a processor; and
a computer-readable storage medium including computer-readable instructions stored therein that, upon execution by the processor, cause the device to calculate a diagnostic score, H, using a mathematical algorithm, wherein the mathematical algorithm comprises equations (1) and (2):
y=exp(X₁−(C₁*age)+(C₂*sex)+(C₃*α2MG)+(C₄*HA)+(C₅*MMP-2)+(C₆*Activin A)) (1)
H=y(1+y) (2)
wherein,
−6.88236≦X₁≦−4.58824,
0.05152≦C₁≦0.07728,
0.45344≦C₂≦0.68016,
0.00896≦C₃≦0.01344,
0.00608≦C₄≦0.00912,
0.00912≦C₅≦0.01368,
0.00216≦C₆≦0.00324,
age is in years,
male sex=1, female sex=0,
α2MG is in mg/dL,
HA is in ng/mL,
MMP-2 is in ng/mL, and
Activin A is in pg/mL.

24. A device configured to nonsurgically predict liver fibrosis in an individual, the device comprising:
an input interface configured to receive data, wherein the data comprise age, sex, and levels of α₂-macroglobulin (α2MG), hyaluronic acid (HA), and Activin A in a sample from the individual,
a processor; and
a computer-readable storage medium including computer-readable instructions stored therein that, upon execution by the processor, cause the device to calculate a diagnostic score, H, using a mathematical algorithm, wherein the mathematical algorithm comprises equations (3) and (2):
y=exp(X₂−(C₇*age)+(C₈*sex)+(C₉*α2MG)+(C₁₀*HA)+(C₁₁*Activin A)) (3)
H=y(1+y) (2)
wherein,
−4.74948≦X₂≦−3.16632,
0.04672≦C₇≦0.07008,
0.38608≦C₈≦0.57912,
0.00880≦C₉≦0.01344,
0.00672≦C₁₀≦0.01008,
0.00912≦C₁₁≦0.01368,
age is in years,
male sex=1, female sex=0,
α2MG is in mg/dL,
HA is in ng/mL, and
Activin A is in pg/mL.
